(19) United States
(12) Patent Application Publication
(10) Pub. No.: US 2007/0174933 A1
(43) Pub. Date: Jul. 26, 2007

(54) Altering levels of anti-nutrient factors in plants

(76) Inventors: Abdelali Hannoufa, Saskatoon (CA); Ulrike A. Schafer, Saskatoon (CA)

Correspondence Address: MORGAN LEWIS & BOCKIUS LLP, 1111 PENNSYLVANIA AVENUE NW, WASHINGTON, DC, 20004, US

(21) Appl. No.: 11/338,849

(22) Filed: Jan. 25, 2006

(51) Int. Cl. A01H 1/00; C12N 15/82

(52) U.S. Cl. 800/285; 435/468

(57) ABSTRACT

A method for reducing the level of one or more than one protein in a plant or a tissue within the plant, and plants with reduced levels of target proteins are provided. The method involves introducing a nucleic acid sequence into the plant, where the nucleic acid sequence comprises a regulatory region operatively associated with a silencing nucleotide sequence. The expression of the silencing nucleotide sequence reduces or eliminates the expression of two or more than two enzymes involved in the synthesis of the one or more than one protein. The reduced level of the one or more than one protein may be determine by comparing the level of the one or more than one protein in the plant, or a tissue of the plant, with a level of the one ore more than one protein in a second plant, or the tissue from the second plant, that does not express the nucleic acid sequence. Plants and seeds obtained from plants with reduced levels of target proteins, and reduced anti-nutrient factors are also provided

### FIELD OF INVENTION

The present invention relates to methods of altering anti-nutrient factors within plants, and plants with altered levels of anti-nutrient factors.

### BACKGROUND OF THE INVENTION

Oil seeds are well known for their high value associated with oil and oil-related products. Proteins obtained from plants, including plant oil seeds for example Canola, are known for their good nutritional qualities. These nutritional properties include, a good balance of essential amino acids, including sulfur binding amino acids, as compared to other plant-based proteins; a low molecular weight of major storage proteins (a characteristic that is usually associated with low antigenicity); and the protein efficiency ratio that in some cases, for example with canola protein, is similar to those of known high quality proteins, such as beef and milk, and better than those of other plant proteins, such as soybean. Despite these high quality characteristics, some plant proteins obtained from oil seeds, including canola protein, are considered a by-product of oil processing, and are used as a low-grade protein source and on a limited scale within livestock feed. Despite the high potential value of canola meal, its market share trails those of other oil seeds, such as soybean. The low value of the canola seed meal is attributed mainly to the high levels of anti-nutritional factors that remain in the seed. These factors include sinapine, fiber and phytate.

Sinapine is found almost exclusively in the seeds of crucifers. Canola seeds contain sinapine at levels ranging from 0.7% to 3%, with about 90% of it present in the embryo (non-hull) fraction. It is the cause for an unpleasant flavour in the meat and milk in animals fed on the canola meal, and the bitter flavor of sinapine results in poor palatability for livestock and fish. Moreover, consumption of sinapine in canola meal by brown-shelled egg layers that are deficient in trimethylamine oxidase imparts a fishy odour on the eggs. Upon consumption of larger amounts, sinapine can also cause serious growth and reproduction problems.

Attempts have been made to reduce sinapine levels via metabolic engineering and plant breeding. A 40% sinapine reduction in seeds of Brassica napus (the main canola species) was achieved by antisense RNA down regulation of ferulic acid 5-hydroxylase (F5H; Nair et al., 2000, Plant Physiol. 123: 1623-1634), and 76% reduction was achieved when sinapate glucosyltransferase (SGT) gene was silenced by RNAi (Husken et al., 2005, Theor. Appl. Genet. 111: 1553-1562). Efforts have also been made towards the identification of low sinapine Brassica germplasm to generate low sinapine varieties by classical plant breeding (Velasco and Möllers, 1998, Crop Sci. 38: 1650-1654).

U.S. Pat. No. 6,501,004 discloses the expression of sense and antisense constructs of ferulate 5-hydroxylase (F5H) in B. napus. Expression of either sense or antisense constructs resulted in a reduction of sinapine levels.

Dietery fiber may represent a significant portion of the meal of seed, for example about a third of canola meal is comprised of dietary fiber. High fiber content can have adverse effects on meal quality, diet digestibility and feed efficiency. Lignin comprises a considerable portion of dietary fiber in the meal, and high levels of it can have adverse effects on meal quality, particularly for fish feed. Fish are very sensitive to high dietary fiber levels. For example, dietary fiber levels of over 10% have a negative impact on growth, diet digestibility, and feed efficiency in trout. Furthermore, high levels of lignin in trees is undesirable for pulp and paper processing where the lignin must be separated from the cellulose fiber used for the production of paper.

Yellow-seeded cultivars of Brassica napus have been developed at Agriculture and Agri-Food Canada's Research Centre in Saskatoon that have lower fibre content of the seed due to the thinner hull of the yellow-seeded types (Relf-Eckstein et al., 2003, In Proceedings of the 11th International Rapeseed Congress, BP9.36 pp. 458-460). Several mutants deficient in the fibre component lignin have been identified in several plant species, including maize and Arabidopsis. In maize, the bm3 mutant has about 12% less lignin than the wild type plant (Grand et al., 1985, Physiol. Veg., 23: 905-911). This mutant is caused by a knockout of the gene coding for caffeic acid o-methyltransferase (COMT; Vignols et al. 1995, Plant Cell, 7: 407-416). In Arabidopsis, the fah1 mutant, caused by T-DNA insertion in the F5H gene (Chapple et al., 1994, Secondary metabolism in Arabidopsis, In Meyerowitz E M and Somerville C R (eds.) Arabidopsis, Cold Spring Harbor Laboratory, Cold Spring harbor, N.Y., pp. 989-1030), has a modified lignin composition (Chapple et al., 1992, Plant Cell. 4: 1413-1424).

Several genes in the phenylpropanoid pathway, including COMT, hydroxycinnamyl alcohol dehydrogenase (CAD) and phenylalanine ammonia lyase (PAL), have been targets for downregulation to reduce lignin content in plants, among which COMT and PAL appeared to be the most promising genes. Transgenic tobacco plants expressing an antisense construct of the alfalfa COMT had a significantly lower lignin content (Ni et al., 1994, Transgen Res., 3: 120-126). Sense suppression of the tobacco PAL gene resulted in up to 3- to 4-fold decrease in lignin content as compared to the wild type (Bate et al., 1994, Proc Natl Acad Sci USA. 91: 7608-7612). On the other hand, repression of CAD expression in tobacco had a negligible impact on lignin content and composition (Haplin et al., 1994, Plant J., 6, 339).

U.S. Pat. No. 6,653,528 discloses the modification of lignin biosynthesis by expressing either a sense and antisense construct of each of O-methyl transferase (OMT), 4-coumarate:CoA lyase (4CL), coumarate 3 hydroxylase (C3H), ferulate 5 hydroxylase (F5H), cinnamoyl-CoA reductase (CCR), PAL, phenylalanine ammonia lyase (PAL), cinnamic acid 4 hydroxylase (C4H). A decrease in lignin concentration (determined by methanol-thioglycoic acid extraction), from 6 to 34% of the control value with was noted with the expression of the antisense construct of OMT. Interestingly, a greater decrease, from 25-45% of the control value, was noted with expression of the sense construct for OMT. Expression of the sense or antisense constructs for the other genes resulted in less of a decrease in lignin concentration when compared to OMT.

U.S. Pat. No. 6,066,780 describes the expression of cinnamyl alcohol dehydrogenase (CAD), CCR, or catechol-O-methyl transferase (COMT), and the effect of their expression on lignin concentration. Expression of the antisense construct of each of these genes resulted in an increase, rather than a decrease, of the TGA extractable products. U.S. Pat. No. 6,465,229 teaches the isolation of caffeoyl-CoA O methylase (CCOMT), however, the effect of expression of this gene in plants was not determined

Phytate is present in canola at levels ranging from 2.0-4.0% in the whole plant, 2.0-5.0% in the oil-free meal and 5.0-7.0% in protein concentrates. Fish and monogastric animals, such as swine and poultry, are unable to digest phytate. The release of phytate increases phosphate in animal waste, and leads to pollution of water systems. Increasing phosphate effluent is a major environmental problem in fresh water aquaculture. Phytate also binds mineral nutrients lowering their bioavailability in the diet, and reducing the nutritional value of the meal.

Several strategies have been used to reduce phytate levels in plant-derived feed. Phytate levels have been reduced by producing low phytate crop varieties, such as rice, maize and barley using mutant selection (Raboy et al. 2001, J. Plant Physiol. 158, 489-497), reducing phytate levels in Arabidopsis seeds through disruption of inositol polyphosphate kinase (Stevenson-Paulik, 2005, Proc. Natl. Acad. Sci. USA 102, 12612-1261), or treating feed with microbial phytases (Cromwell et al. 1995, J. Anim. Sci., 73: 2000-2008). WO 2005/014794 discloses the reduction of phytate levels in plants by modifying inositol phosphate kinase (Ipk1 and Ipk2) expression. Reduction of phosphorous manure has been obtained by engineering transgenic animals to produce heterologous phytases in their salivary glands (Golovan et al., 2001, Nat. Biotech, 19: 741-745). However, treatment of feed with microbial phytases is an expensive process, and is cost-effective only in regions where high penalties for disposing manure with high phosphorus content exist. Engineering animals to produce phytase in salivary glands is currently impractical with most animals. Other strategies, such as mutant screening and genetic engineering, have yet to yield B. napus varieties with substantially reduced levels of phytate.

### SUMMARY OF THE INVENTION

The present invention relates to methods of altering anti-nutrient factors within plants, and plants with altered levels of anti-nutrient factors.

According to the present invention there is provided a method (A) for reducing the level of one or more than one protein in a plant or a tissue within the plant comprising,

i) introducing a nucleic acid sequence into the plant, the nucleic acid sequence comprising a regulatory region operatively associated with a silencing nucleotide sequence, wherein expression of the silencing nucleotide sequence reduces or eliminates the expression of two or more than two enzymes involved in the synthesis of the one or more than one protein, and

ii) expressing the silencing nucleotide sequence within the plant or a tissue within the plant, to reduce the level of the one or more than one protein in the plant or within a tissue of the plant, the reduced level of the one or more than one protein is determined by comparing the level of the one or more than one protein in the plant, or a tissue of the plant, with a level of the one ore more than one protein in a second plant, or the tissue from the second plant, that does not express the silencing nucleic acid sequence.

The present invention also provides the method as just defined (Method A), wherein in the step of introducing, the nucleotide sequence is introduced into the plant by transformation. Alternatively, the nucleotide sequence may be introduced into the plant by crossing the plant with a second plant, the second plant comprising the nucleotide sequence.

The present invention also provides the method as described above (method A) wherein the regulatory region is selected from the group consisting of a constitutive regulatory region, an inducible regulatory region, a developmentally regulated regulatory region, and a tissue specific regulatory region. The regulatory region is preferably a tissues specific regulatory region.

The present invention pertains to the method described above (method A), wherein the silencing nucleotide sequence is selected from the group consisting of an antisense RNA encoding nucleotide sequence, a ribozyme encoding sequence, and an RNAi encoding nucleotide sequence. Preferably, the silencing nucleotide sequence is a gene fusion. Furthermore, the gene fusion may comprise nucleic acid sequences encoding from two to five gene sequences.

The present invention also pertains to the method as described above (method A) wherein the protein is involved in the synthesis of an anti-nutrient factor in seed tissue. The anti-nutrient factor may be selected from the group consisting of sinapine, phytate, fiber, and lignin. Furthermore, the two or more than two enzymes are involved in a pathway of phenylpropanoid biosynthesis leading to sinapine synthesis, a pathway of phenylpropanoid biosynthesis leading to lignin synthesis, a pathway of phytate biosynthesis, or a combination thereof.

According to the present invention there is provided a method (B) for reducing the level of one or more than one protein involved in the synthesis of an anti-nutrient factor in a plant or a tissue within the plant comprising,

i) introducing a nucleic acid sequence into the plant, the nucleic acid sequence comprising a regulatory region operatively associated with a silencing nucleotide sequence, wherein expression of the silencing nucleotide sequence reduces or eliminates the expression of two or more than two enzymes involved in the synthesis of the one or more than one protein, and

ii) expressing the silencing nucleotide sequence within the plant or a tissue within the plant, to reduce the level of the one or more than one protein in the plant or within a tissue of the plant, the reduced level of the one or more than one protein is determine by comparing the level of the one or more than one protein in the plant, or a tissue of the plant, with a level of the one ore more than one protein in a second plant, or the tissue from the second plant, that does not express the nucleic acid sequence,

wherein the anti-nutrient factor is sinapine, and the two or more than two enzymes are involved in a pathway of phenylpropanoid biosynthesis.

The present invention also provides the method as described above (method B), wherein the two or more than two enzymes are selected from the group consisting of phenylalanine ammonia lyase (PAL), cinnamate 4 hydroxylase (C4H), coumarate 3 hydroxylase (C3H), caffeic acid-/5-hydroxyferulic acid O-methyltransferase (COMT; also referred to as O-methyl transferase, OMT), ferulic acid hydroxylase (FAH), sinapate:UDP-glucose sinapoyltransferase (SGT), sinapoylglucose:malate sinapoyltransferase (SMT), sinapoylglucose:choline sinapoyltransferase (SCT), S-adenosylmethionine synthase (SAMS).

Preferably, the two or more than two enzymes (in Method B) are FAH and SCT. Furthermore, the silencing nucleotide sequence is selected from the group consisting of an antisense RNA nucleotide sequence encoding sequence, a ribozyme encoding sequence, and an RNAi encoding nucleotide sequence.

According to the present invention there is provided a method (C) for reducing the level of one or more than one protein involved in the synthesis of an anti-nutrient factor in a plant or a tissue within the plant comprising,

i) introducing a nucleic acid sequence into the plant, the nucleic acid sequence comprising a regulatory region operatively associated with a silencing nucleotide sequence, wherein expression of the silencing nucleotide sequence reduces or eliminates the expression of two or more than two enzymes involved in the synthesis of the one or more than one protein, and

ii) expressing the silencing nucleotide sequence within the plant or a tissue within the plant, to reduce the level of the one or more than one protein in the plant or within a tissue of the plant, the reduced level of the one or more than one protein is determine by comparing the level of the one or more than one protein in the plant, or a tissue of the plant, with a level of the one ore more than one protein in a second plant, or the tissue from the second plant, that does not express the nucleic acid sequence,

wherein the anti-nutrient factor is lignin, and the two or more than two enzymes are involved in a pathway of phenylpropanoid biosynthesis.

The present invention also provides the method as described above (method C), wherein the two or more than two enzymes are selected from the group consisting of cinnamic acid 4 hydroxylase (C4H), coumaric acid 3 hydroxylase (C3H), caffeic acid O-methyl transferase (COMT), ferulic acid hydroxylase (FAH), S-adenosylmethionine synthase (SAMS), 4-coumarate:CoA lyase (4CL), and cinnamoyl-CoA reductase (CCR).

Preferably, the two or more than two enzymes (in Method C) are selected from the group consisting of FAH-COMT, C3H-C4H, and 4CL-CCR. Furthermore, the silencing nucleotide sequence is selected from the group consisting of an antisense RNA nucleotide sequence encoding sequence, a ribozyme encoding sequence, and an RNAi encoding nucleotide sequence.

According to the present invention there is provided a method (D) for reducing the level of one or more than one protein involved in the synthesis of an anti-nutrient factor in a plant or a tissue within the plant comprising,

i) introducing a nucleic acid sequence into the plant, the nucleic acid sequence comprising a regulatory region operatively associated with a silencing nucleotide sequence, wherein expression of the silencing nucleotide sequence reduces or eliminates the expression of two or more than two enzymes involved in the synthesis of the one or more than one protein, and

ii) expressing the silencing nucleotide sequence within the plant or a tissue within the plant, to reduce the level of the one or more than one protein in the plant or within a tissue of the plant, the reduced level of the one or more than one protein is determine by comparing the level of the one or more than one protein in the plant, or a tissue of the plant, with a level of the one or more than one protein in a second plant, or the tissue from the second plant, that does not express the nucleic acid sequence, wherein the anti-nutrient factor is phytate, and the two or more than two enzymes are involved in a pathway of phytate biosynthesis.

The present invention also provides the method as described above (method D), wherein the two or more than two enzymes are selected from the group consisting of inositol 1,3,4-trisphosphate 5/6-kinase (IP3K), IP6K, myo-inositol hexaphosphate kinase, 1-phosphatidylinositol-4,5-bisphosphate phosphodiesterase (PIBP PDE), phosphatidylinositol phophatidylcholine transfer protein (PI/PC TP), inositol polyphosphate 5-phosphatase II (IPP), phosphatidylinositol-4-phosphate 5-kinase (PIKa), CDP-diacylglycerol-inositol 3-phosphatidyltransferase (phosphatidylinositol synthase, PIS), inositol polyphosphate 6-/3-/5-kinase 2b (IPK2a & IPK2b), inositol polyphosphate 5′-phosphatase I (IPP), Myo-inositol-1-phosphate synthase (MIP), phosphatidylinositol kinase (PIPK), phosphatidylinositol 3-kinase (PI3K), Myo-inositol monophosphatase (MIM), and phosphatidylinositol 3- and 4-kinase (PIKb).

Preferably, the two or more than two enzymes (in Method D) are selected from the group consisting of IP3K-IP6K and PIKa-PIKb. Furthermore, the silencing nucleotide sequence is selected from the group consisting of an antisense RNA nucleotide sequence encoding sequence, a ribozyme encoding sequence, and an RNAi encoding nucleotide sequence.

The present invention also provides a construct comprising a silencing nucleotide sequence, the silencing nucleotide sequence encoding two or more than two sequences that reduce or inhibit the expression of two or more that two enzymes involved in the synthesis of the one or more than one protein leading to sinapine biosynthesis, lignin biosynthesis, phytate biosynthesis, or a combination thereof.

The present invention also pertains to a plant comprising a nucleic acid sequence comprising a regulatory region operatively associated with a silencing nucleotide sequence, the silencing nucleotide sequence reducing or eliminating the expression of two or more than two enzymes involved in the synthesis of the one or more than one protein leading to sinapine biosynthesis, lignin biosynthesis, phytate biosynthesis, or a combination thereof.

The present invention also provides a seed that is characterized as having reduced levels of one or more than one anti-nutrient compounds. Examples of an anti-nutrient compound include intermediates within the phenylpropanoid pathway, or products of the phenylpropanoid pathway including sinapine and lignin, the phytate biosynthetic pathway, including phytate. The level of the anti-nutrient compound may be reduced by about 10% to about 100%, or any amount therebetween, when compared to the level of the same anti-nutrient compound obtained from a second plant that does not express a silencing nucleotide sequence. For example, the anti-nutrient compound may be reduced by from about 10% to about 60% or any amount therebetween, about 10% to about 50% or any amount therebetween, about 10% to about 40% or any amount therebetween, or from about 10% to about 30%, or any amount therebetween, or about 10% to about 20% or any amount therebetween.

The present invention also provides a method (Method E) for reducing the level of one or more than one protein within a plant or a tissue within the plant comprising, expressing a nucleotide sequence within the plant or a tissue within the plant, the nucleic acid sequence comprising a regulatory region operatively associated with a silencing nucleotide sequence, where expression of the silencing nucleotide sequence reduces or eliminates the expression of two or more than two enzymes involved in the synthesis of the one or more than one protein leading to sinapine biosynthesis, lignin biosynthesis, phytate biosynthesis, or a combination thereof, the reduced level of the one or more than one protein determined by comparing the level of the protein in the plant, or a tissue of the plant, with a level of the protein in a second plant, or the tissue from the second plant, that does not express the nucleic acid sequence.

The present invention also provides a method (Method F) for reducing the level of one or more than one protein in a plant or a tissue within the plant comprising,

i) providing a plant comprising a nucleic acid sequence, the nucleic acid sequence comprising a regulatory region operatively associated with a silencing nucleotide sequence, wherein expression of the silencing nucleotide sequence reduces or eliminates the expression of two or more than two enzymes involved in the synthesis of the one or more than one protein, and

ii) expressing the silencing nucleotide sequence within the plant or a tissue within the plant, to reduce the level of the one or more than one protein in the plant or within a tissue of the plant, the reduced level of the one or more than one protein is determine by comparing the level of the one or more than one protein in the plant, or a tissue of the plant, with a level of the one ore more than one protein in a second plant, or the tissue from the second plant, that does not express the silencing nucleic acid sequence.

Unlike the limited scope of conventional breeding, molecular approaches to reduce lignin have much wider applications in plant biotechnology. Silencing of COMT, SAMS, C3H and C4H can alter lignin levels not only in Brassica oilseeds, but also potentially in other plants where high levels of lignin are undesirable, e.g. woody plants for the pulp and paper applications.

This invention is aimed at improving seed meal, for example canola meal by reducing the levels of one or more than one antinutritional factor (ANF), including but not limited to sinapine, phytate, lignin, and a combination of sinapine, phytate and lignin. This may be achieved by impairing the expression of rate limiting genes in the biosynthesis pathways of these metabolites. Mutant plant lines with knockouts in genes affecting the metabolism of phenylpropanoids, phytate, or phenylpropanoids and phytate, were isolated and characterized, and constructs to silence key genes in these pathways were engineered The approach is exemplified using B. napus, however, other plants may also be modified using the methods as described herein, for example, but not limited to plants within the Crucifereae, tobacco, soybean, woody trees used in pulp and paper, for example, spruce, pine, Douglas fir, Alpine fir, larch, poplar, eucalyptus and the like.

This summary of the invention does not necessarily describe all features of the invention.

### BRIEF DESCRIPTION OF THE DRAWINGS

These and other features of the invention will become more apparent from the following description in which reference is made to the appended drawings wherein:

FIG. 1 shows a schematic of the phenylpropanoid pathway. PAL: phenylalanine ammonia lyase; C4H: cinnamate 4 hydroxylase; C3H: coumarate 3 hydroxylase; OMT: O-methyl transferase; FAH: ferulic acid hydroxylase; SGT: sinapate:UDP-glucose sinapoyltransferase SMT: Sinapate:malate sinapoyltransferase; SCT: sinapolyglucose: choline sinapoyltransferase; 4CL:4-coumarate:CoA lyase; CCR: cinnamoyl-CoA reductase CAD: cinnamyl alcohol dehydrogenase; POD: peroxidase.

FIG. 2 shows vectors, engineering of antisense and RNAi constructs, and cloning strategy for fusion products. FIG. 2a shows a generic pGSA series vector used for engineering of RNAi constructs. Vector pGSA1252 contains a pMas2′ driven BAR gene for BASTA selection in plants, and vector pGSA1285 contains a pMas2′ driven NptII gene for kanamycin selection in plants. FIG. 2b shows an outline of a strategy for cloning fusion products (see Examples for details). FIG. 2c shows a schematic diagram of p72-148, in pBluescript (napin::SCT-FAH AS; see Examples for details). FIG. 2d shows a schematic diagram of p79-103, a plant transformation vector having a NOS driven BAR gene for BASTA selection in plants. FIG. 2e shows a schematic diagram of p72-515 (see Examples for details). FIG. 2f shows a schematic diagram of an RNAi intermediate using a GUS linker (see Examples for details). FIG. 2g shows a cloning strategy for placing GUS linker with RNAi into plant transformation vector (pGSA series vector). FIG. 2h shows a schematic diagram of p 72-512 (SEQ ID NO: 24).

FIG. 3 shows the levels of sinapine in seeds of wild type and knockout mutant lines in A. thaliana. Col wt: wild type, knockout mutant lines (see Examples).

FIG. 4 shows reduction of sinapine in seeds of transgenic lines of B. napus expressing an antisense construct comprising SCT. FIG. 4a shows a schematic diagram of CaMV35S::SCT RNAi construct, including a CaMV35S promoter, a sense and antisense SCT sequence separated by a linker fragment. FIG. 4b shows levels of sinapine in seeds obtained from transgenic (DE192 to DE329) and wild type control (DH12075) lines of Brassica napus. FIG. 4c shows Northern blot analysis of total RNA isolated from seeds of the offspring of DE324 (RS1859 to RS1862 expressing CaMV35S::SCT RNAi construct) collected at various days after flowering (DAF) and probed with the SCT-specific exon. FIG. 4d shows quantification of silencing levels. Relative densities generated by Northern blot analysis were expressed as a percent of the densities of the corresponding 28s rRNA bands on the ethidium bromide-stained gel. DH12075: wild type plants; RS1859 to RS1862 (offspring of DE324) transgenic lines expressing the CaMV35S::SCT RNAi construct.

FIG. 5 shows reduction of sinapine in seeds of transgenic lines of B. napus expressing an antisense construct comprising a FAH::SCT gene fusion, under control of a napin promoter. FIG. 5a shows a schematic diagram of the napin::FAH-SCT antisense construct. FIG. 5b shows levels of sinapine in seeds obtained from transgenic (DE355 to DE384) and wild type control lines (DH12075) of Brassica napus. FIG. 5c shows Northern blot analysis of total RNA isolated from seeds of the offspring of DE362 (RS1863 to RS1866), DE368 (RS1868), and DE373 (RS1869 to RS1872), collected at various days after flowering (DAF) probed with the SCT-specific exon. FIG. 5d shows quantification of silencing levels. Relative densities generated by Northern blot analysis were expressed as a percent of the densities of the corresponding 28s rRNA bands on the ethidium bromide-stained gel. DH12075: wild type plants; RS1863 to RS1866 (offspring of DE362), RS1868 (offspring of DE368), RS1869 to RS1872 (offspring of DE373): transgenic lines expressing the napin::FAH-SCT antisense construct.

FIG. 6 shows reduction of sinapine in transgenic Brassica napus seeds expressing an antisense construct comprising a C3H-C4H gene fusion, under control of a cruciferin promoter. FIG. 6a shows a schematic diagram of the cruciferin::C3H-C4H antisense construct. FIG. 6b shows levels of sinapine in seeds obtained from transgenic (DE443 to DE599) plants expressing the cruciferin::C3H-C4H antisense construct and wild type control lines (DH12075).

FIG. 7 shows reduction of sinapine in transgenic Brassica napus seeds expressing an RNAi construct comprising a C3H-C4H gene fusion, under control of a CaMV35S promoter. FIG. 7a shows a schematic diagram of the CaMV35S::C3H-C4H RNAi construct. FIG. 7b shows levels of sinapine in seeds obtained from transgenic (DE268 to DE496) plants expressing the CaMV35S::C3H-C4H RNAi construct, and wild type control line (DH12075).

FIG. 8 shows reduction of sinapine levels in transgenic Brassica napus seeds expressing an RNAi construct comprising a SAMS, under control of a napin promoter. FIG. 8a shows a schematic diagram of the napin::SAMS RNAi construct. FIG. 8b shows levels of sinapine in seeds obtained from transgenic (DE295 to DE318) plants expressing the napin::SAMS RNAi construct, and wild type control line (DH12075).

FIG. 9 shows reduction of lignin levels in transgenic Brassica napus seeds expressing an RNAi construct comprising COMT under the control of a cruciferin promoter. FIG. 9a shows a schematic diagram of the Cruciferin::COMT RNAi construct. FIG. 9b shows lignin levels in seeds expressed as a percent of wild type. DH12075: Wild type control lines; Doelolla: low lignin Brassica carinata lines; DE132 to DE187: transgenic lines expressing Cruciferin::COMT RNAi construct.

FIG. 10 shows reduction of lignin levels in transgenic Brassica napus seeds expressing FAH-COMT RNAi. FIG. 10a shows a schematic diagram of the CaMV35S::FAH-COMT RNAi construct. FIG. 10b shows lignin levels expressed as a percent of wild type lignin levels. DH12075: Wild type lines; Doelolla: low lignin Brassica carinata lines, DE240 to DE472: transgenic lines expressing the CaMV35S::FAH-COMT RNAi construct.

FIG. 11 shows reduction of lignin levels in transgenic Brassica napus seeds expressing SAMS RNAi. FIG. 11a shows a schematic diagram of the napin::SAMS RNAi construct. FIG. 11b shows lignin levels expressed as a percent of wild type lignin levels. DH12075: wild type plants; Doelolla: low lignin Brassica carinata lines: DE295 to DE318: transgenic lines expressing the napin::SAMA RNAi construct.

FIG. 12 shows reduction of lignin levels in transgenic Brassica napus seeds expressing antisense COMT. FIG. 12a shows a schematic diagram of the napin::COMT antisense construct. FIG. 12b shows lignin levels, expressed as a percent of wild type lignin levels. DH12075: wild type line; Doelolla: low lignin Brassica carinata line; DE36 to DE111 transgenic lines expressing the napin::COMT antisense construct.

FIG. 13 shows reduction of lignin levels in transgenic Brassica napus seeds expressing C3H-C4H RNAi. FIG. 13a shows a schematic diagram of the 35S::C3H-C4H RNAi construct. FIG. 13b shows lignin levels, expressed as a percent of wild type lignin levels. DH12075: Wild type plants; Doelolla: low lignin Brassica carinata line; DE268 to DE496: transgenic lines expressing the 35S::C3H-C4H RNAi construct.

FIG. 14 shows reduction of sinapine in transgenic Brassica napus seeds expressing an RNAi construct comprising the COMT gene under control of a CaMV35S promoter. FIG. 14a shows a schematic diagram of the CaMV35S::COMT RNAi construct. FIG. 14b shows levels of sinapine in seeds obtained from transgenic (DE114 to DE191) plants expressing the CaMV35S::COMT RNAi construct, and wild type control line (DH12075).

FIG. 15 shows reduction of sinapine in transgenic Brassica napus seeds expressing an antisense construct comprising the COMT gene under the control of a cruciferin promoter. FIG. 15a shows a schematic diagram of the cruciferin::COMT antisense construct. FIG. 15b shows levels of sinapine in seeds obtained from transgenic (AB411 to AB438) plants expressing the cruciferin::COMT antisense construct and wild type control lines (DH12075).

FIG. 16 shows reduction of lignin levels in transgenic Brassica napus seeds expressing antisense COMT. FIG. 16a shows a schematic diagram of the cruciferin::COMT antisense construct. FIG. 16b shows lignin levels, expressed as a percent of wild type lignin levels. DH12075: wild type line; Doelolla: low lignin Brassica carinata line; AB411 to AB438 transgenic lines expressing the cruciferin::COMT antisense construct.

FIG. 17 shows reduction of sinapine in transgenic Brassica napus seeds expressing an RNAi construct comprising a FAH-COMT gene fusion, under control of a CaMV35S promoter. FIG. 17a shows a schematic diagram of the CaMV35S::FAH-COMT RNAi construct. FIG. 17b shows levels of sinapine in seeds obtained from transgenic (DE240 to DE472) plants expressing the CaMV35S::FAH-COMT RNAi construct, and wild type control line (DH12075).

FIG. 18 shows a schematic of the Inositol phosphate pathway (phytate biosynthetic pathway).

### DETAILED DESCRIPTION

The present invention relates to methods of altering one or more than one anti-nutrient factor within plants, and plants with altered levels of one or more than one anti-nutrient factor. Examples of anti-nutrient factors that may be modified include sinapine, phytate, and lignin.

The following description is of an exemplary embodiment.

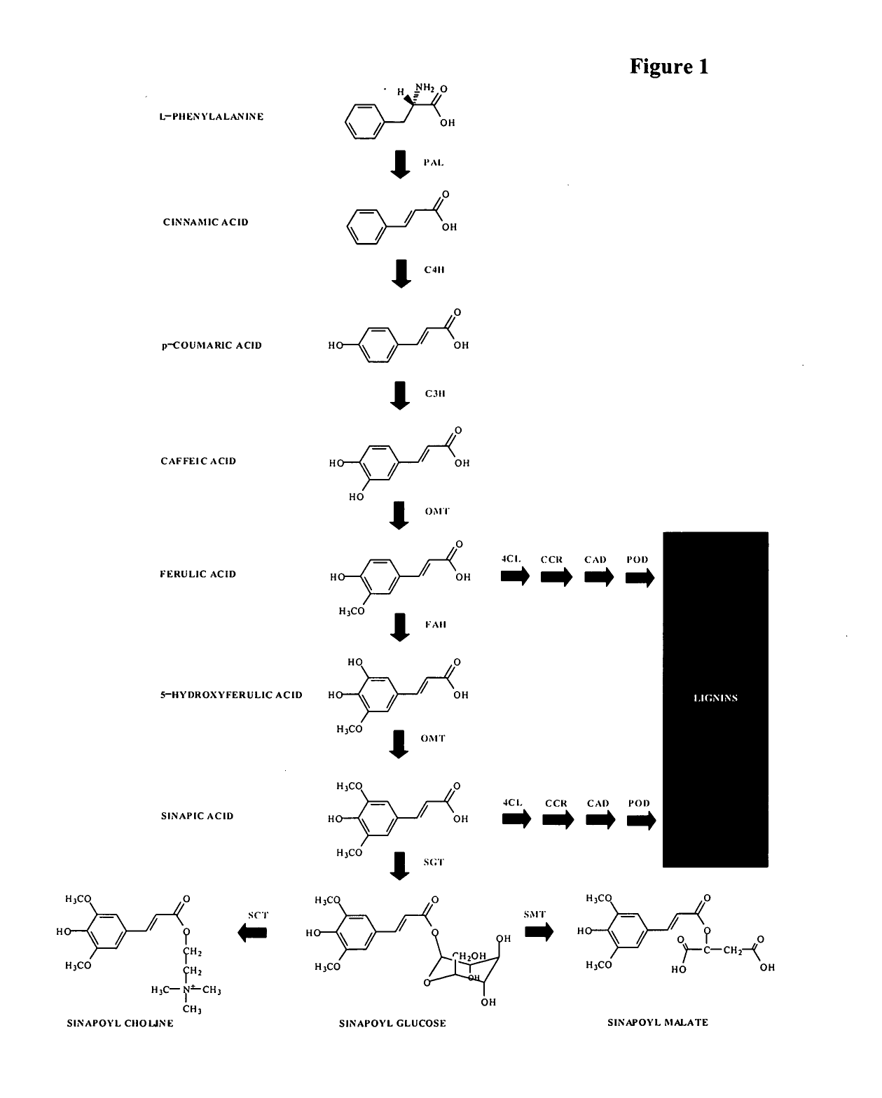

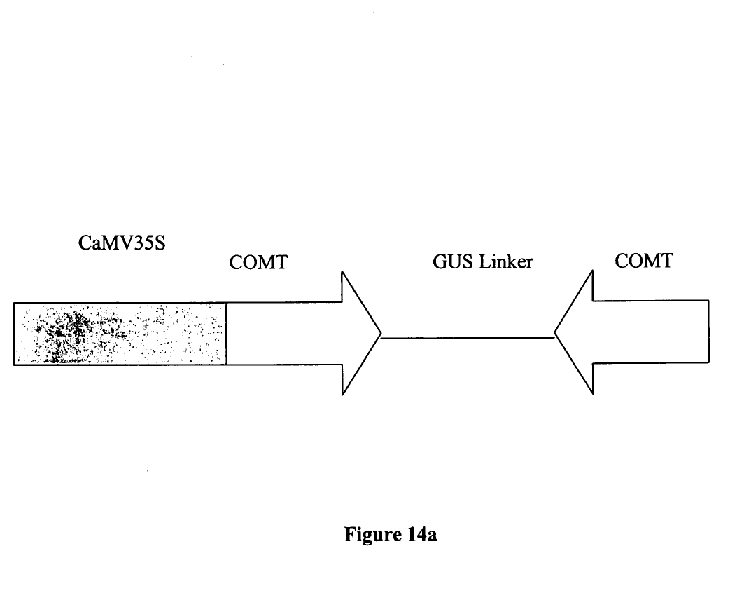

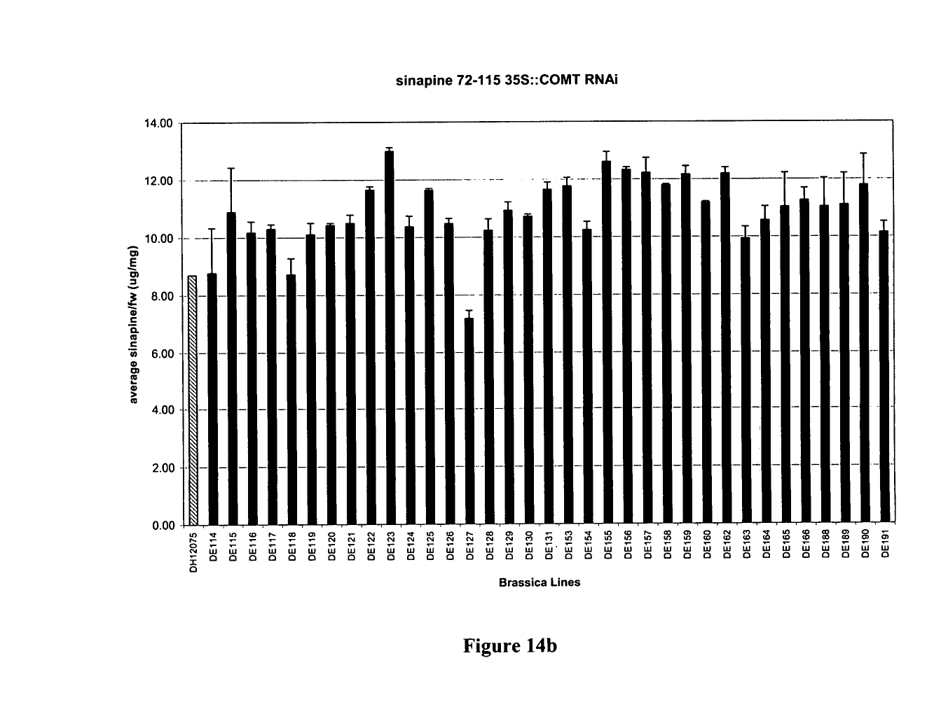

The present invention provides methods and plants with reduced anti-nutrient factors. These anti-nutrient factors may include, but are not limited to products of the phenylpropanoid pathway (see FIG. 1), for example sinapine, and lignin, the phytate biosynthesis (see FIG. 14), for example phytate, or a combination of products produced within, or by, these pathways. Additonally, even though not shown in FIG. 1, S-adenosyl methionine Synthase (SAMS) catalyses the synthesis of the methyl donor SAM (S-adenosyl methionine) from methionine and affects methylation reactions catalyzed by COMT (or OMT) in the phenylpropanoid pathway.

The present invention therefore provides a method for reducing the level of one or more than one protein in a plant or a tissue within the plant comprising,

i) introducing a nucleic acid sequence into the plant, the nucleic acid sequence comprising a regulatory region operatively associated with a silencing nucleotide sequence, wherein expression of the silencing nucleotide sequence reduces or eliminates the expression of one, or two, or more than one, or two, enzymes involved in the synthesis of the one or more than one protein, and

ii) expressing the silencing nucleotide sequence within the plant or a tissue within the plant, to reduce the level of the one or more than one protein in the plant or within a tissue of the plant, the reduced level of the one or more than one protein is determined by comparing the level of the one or more than one protein in the plant, or a tissue of the plant, with a level of the one ore more than one protein in a second plant, or the tissue from the second plant, that does not express the nucleic acid sequence.

The level of the one or more than one protein may be reduced by about 10% to about 100%, or any amount therebetween, where compared to the level of the same one or more than one protein obtained from a second plant that does not express the nucleotide sequence. For example, the protein may be reduced by from about 10% to about 60% or any amount therebetween, about 10% to about 50% or any amount therebetween, about 10% to about 40% or any amount therebetween, or from about 10% to about 30%, or any amount therebetween, or about 10% to about 20% or any amount therebetween.

The regulatory region may be a constitutive regulatory region, an inducible regulatory region, a developmentally regulated regulatory region, and a tissue specific regulatory region.

By the term “expression” it is meant the production of a functional RNA, protein or both, from a gene or transgene.

By “reduction of gene expression” it is meant the reduction in the level of mRNA, protein, or both mRNA and protein, encoded by a gene or nucleotide sequence of interest. Reduction of gene expression may arise as a result of the lack of production of full length RNA, for example mRNA, or through cleaving the mRNA, for example with a ribozyme (e.g. see Methods in Molecular Biology, vol 74 Ribozyme Protocols, P. C. Turner, ed, 1997, Humana Press), or RNAi (e.g. see Gene Silencing by RNA Interference, Technology and Application, M. Sohail ed, 2005, CRC Press), or otherwise reducing the half-life of RNA, using antisense (e.g. see Antisense Technology, A Practical Approach, C. Lichtenstien and W. Nellen eds., 1997, Oxford University Press), ribozyme, or RNAi techniques.

A “silencing nucleotide sequence” refers to a sequence that when transcribed results in the reduction of expression of a target gene, or of two or more than two target genes, for example, 1, 2, 3, 4, 5, 6, 7, 8, 9, 10 target genes, or any number of target genes therebetween. A silencing nucleotide sequence may involve the use of antisense RNA, a ribozyme, or RNAi, targeted to a single target gene, or the use of antisense RNA, ribozyme, or RNAi, comprising two or more than two sequences that are linked or fused together and targeted to two or more than two target genes. When transcribed the product of the silencing nucleotide sequence may target one, or it may target two or more than two, of the target genes. When two or more than two sequences are linked or fused together, these sequences may be referred to as gene fusions, or gene stacking. It is within the scope of the present invention that gene fusions may comprise 2, 3, 4, 5, 6, 7, 8, 9, 10 nucleotide sequences, or any number therebetween, that are fused or linked together. The fused or linked sequences may be immediately adjacent each other, or there may be linker fragment between the sequences.

Reduction in the expression of a target gene, or two or more than two target genes, results in the reduced synthesis of a protein encoded by the target sequence, or the two or more proteins encoded by the two or more than two target sequences. Preferably the protein is involved in the synthesis of an anti-nutrient factor in plant tissue (e.g. stem, leaf, root, flower), including seed tissue, but it may also be involved in reducing fiber content in plant tissue (e.g. stem, leaf, root). Examples of anti-nutrient factors include, but are not limited to sinapine, phytate, fiber, and lignin, and the protein may be involved in a pathway of phenylpropanoid biosynthesis leading to sinapine synthesis, a pathway of phenylpropanoid biosynthesis leading to lignin synthesis, a pathway of phytate biosynthesis, or a combination thereof.

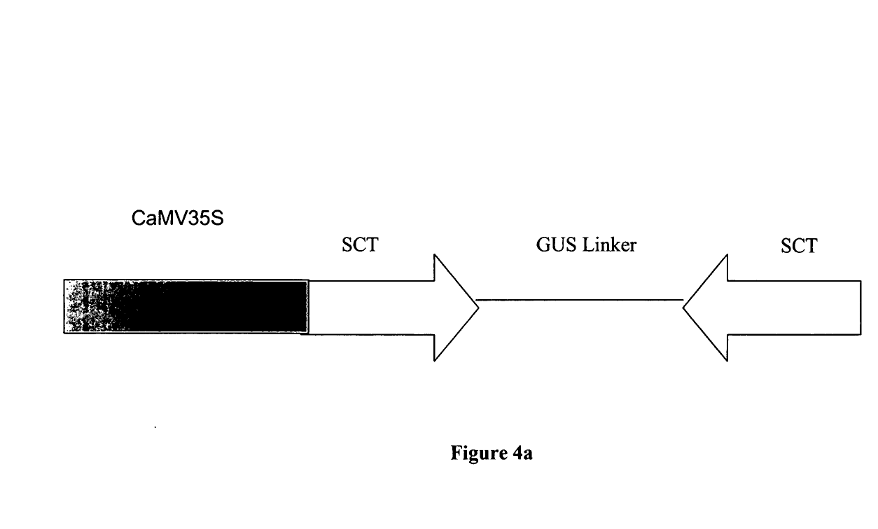

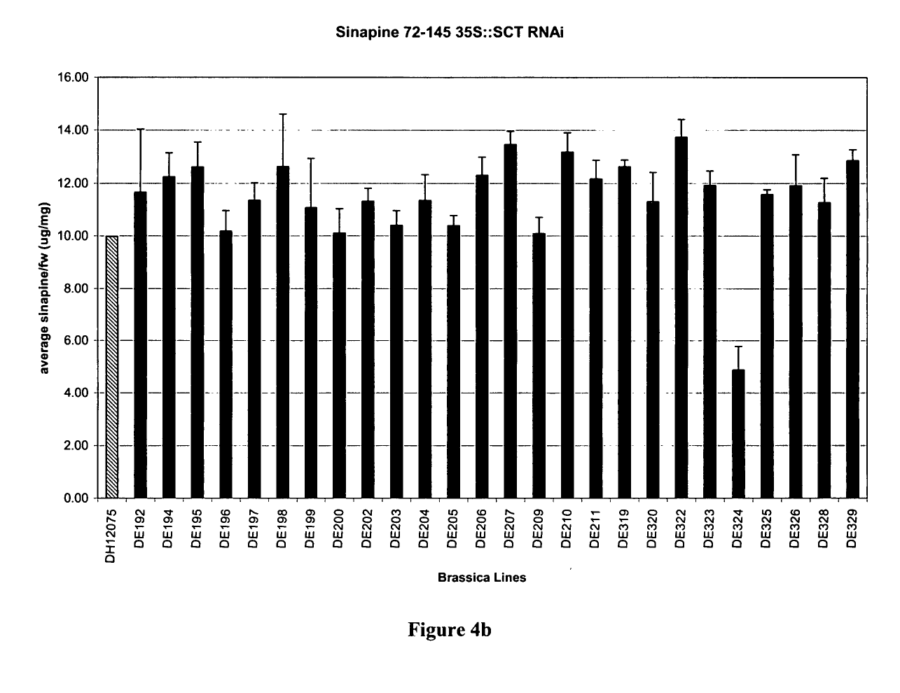

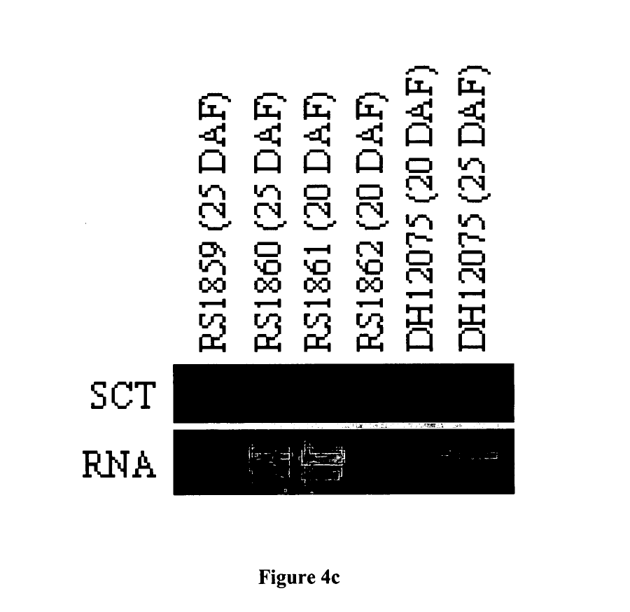

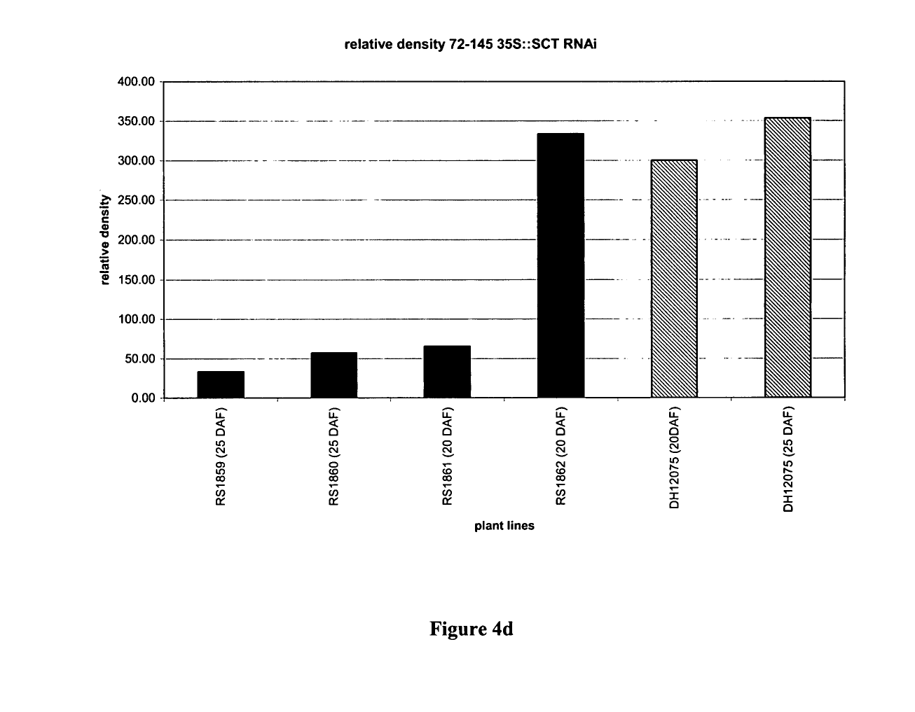

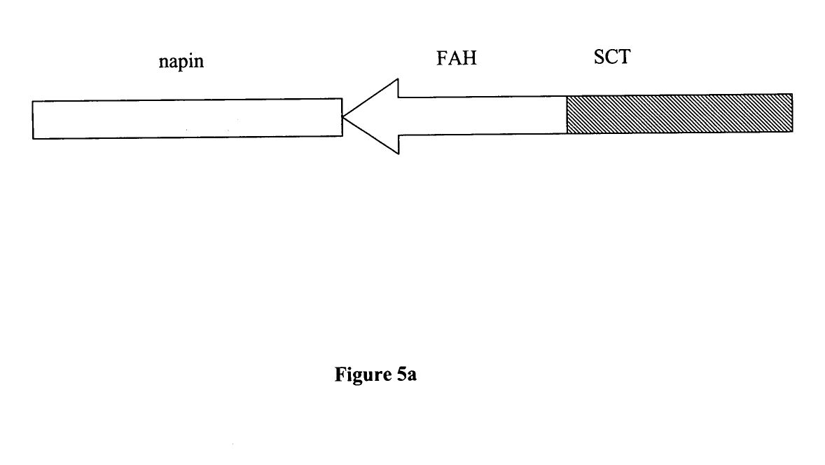

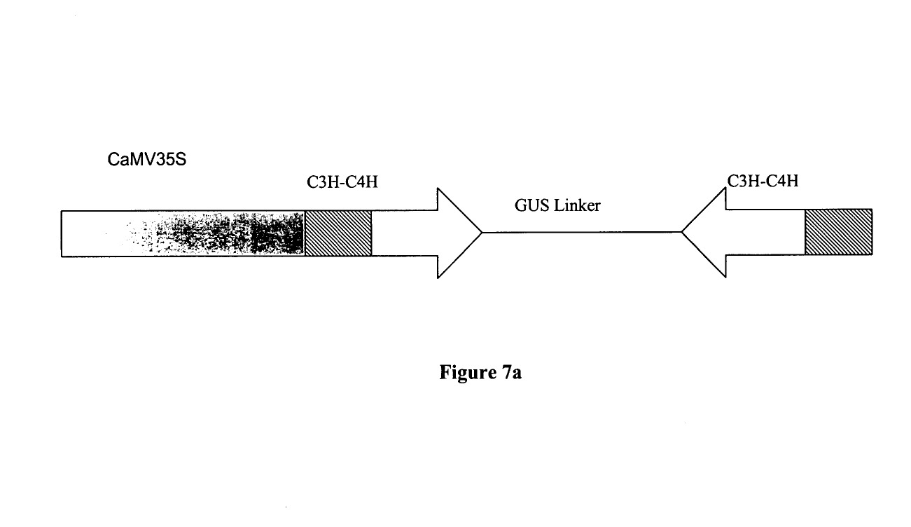

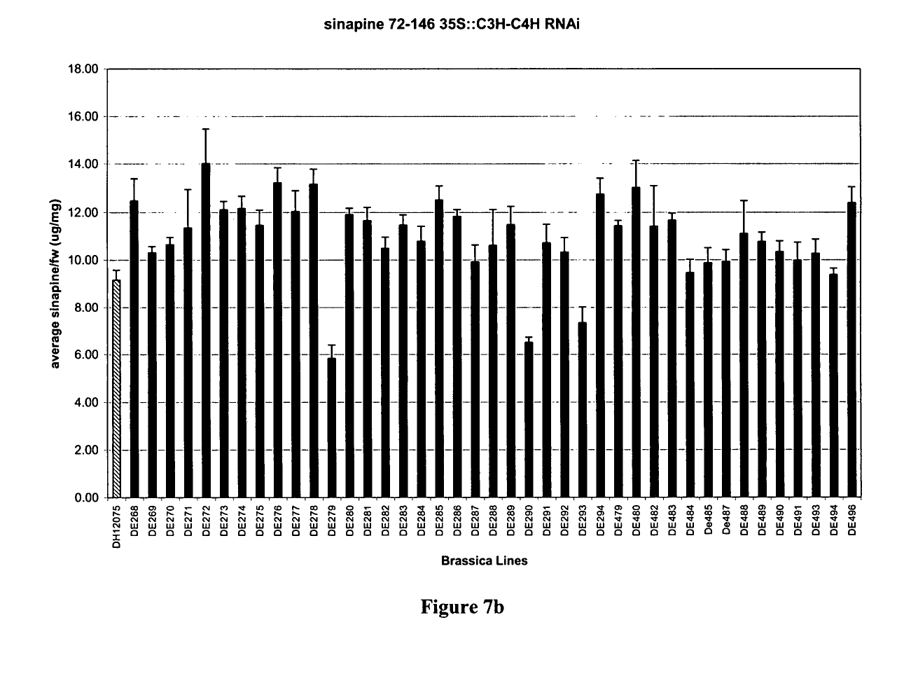

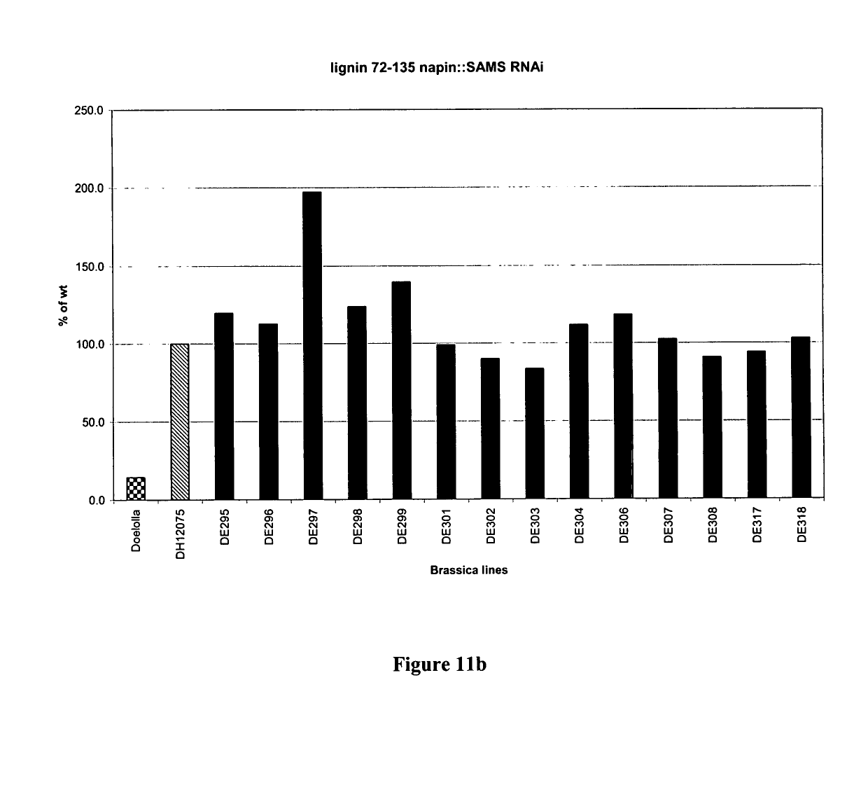

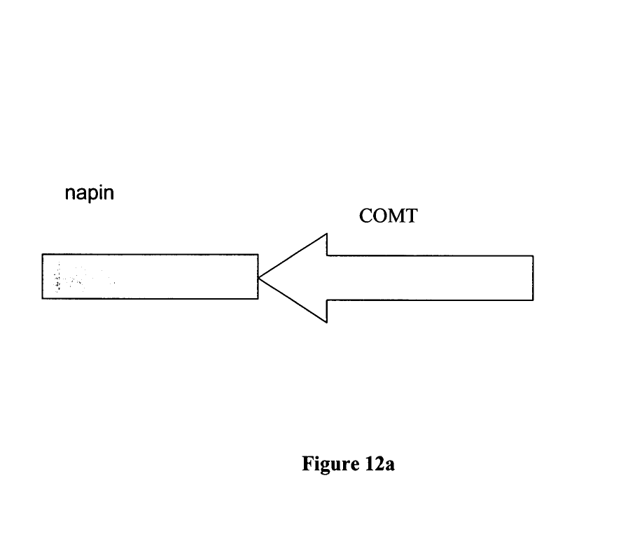

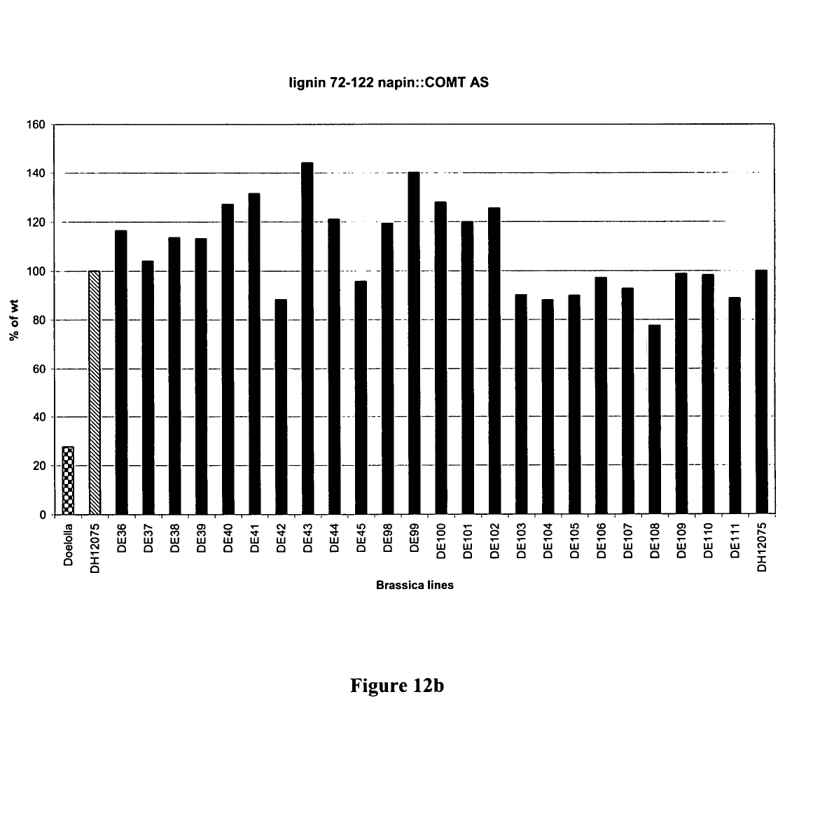

Non-limiting examples of a silencing nucleotide sequence include the antisense sequence napin::COMT AS (e.g. p72-122, see FIG. 12a) or the gene fusion: napin::FAH-SCT AS (e.g. p72-148; see FIG. 5a), the RNAi sequence CaMV35S::SCT RNAi (e.g. p72-145; see FIG. 4a), or the gene fusion CaMV35S::C3H-C4H RNAi (e.g. p72-146; see FIG. 7a). Additional non-limiting examples of gene fusions are provided in the figures and examples herein.

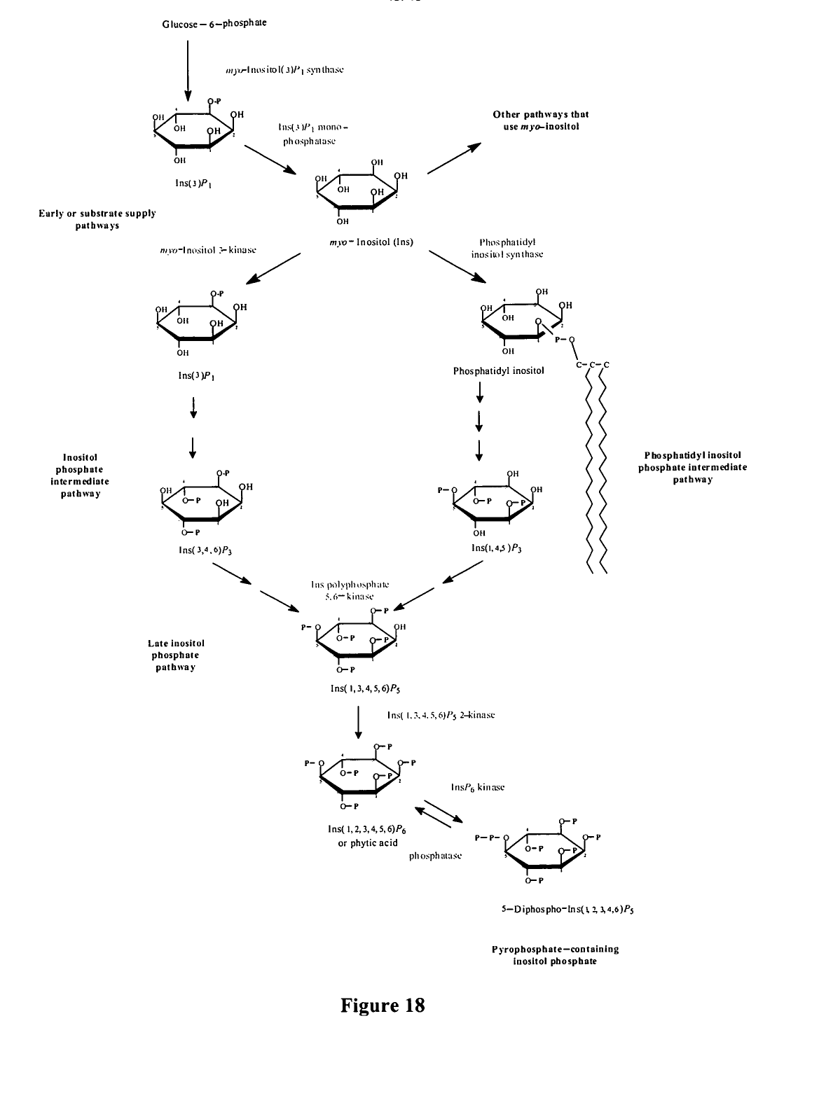

Nucleotide sequences used for gene fusions may comprise sequences that encode proteins, that are involved in the same biosynthetic pathway, for example two or more enzymes involved in the phenylpropanoid pathway (FIG. 1), or the pathway leading to phytate biosynthesis (see FIG. 18), or a combination thereof Therefore, gene fusions may comprise one or more that one nucleotide sequence encoding one or more than one enzyme involved in for example, sinapine and phytate synthesis, sinapine and lignin biosynthesis, lignin and phytate biosynthesis, or sinapine, lignin and phytate biosynthesis. In complex biosynthetic pathways, it may be preferred to use gene fusions comprising from 2 to about 10 sequences, or nay number of sequences therebetween, to ensure that multiple steps of the pathway are interrupted. Similarly, gene stacking using from about 2 to about 10 sequences, or any number therebetween, may be used to interrupt multiple steps of one or more than one pathway, including phenylpropanoid biosynthesis, phytate biosynthesis, and both phenylpropanoid and phytate biosynthesis. In this way, a reduction of anti-nutrient factors may be achieved for several compounds.

Plants comprising two or more than two nucleic acids, including gene fusions, or combinations of silencing nucleic acid sequences, may be introduced into a plant using standard techniques, for example, but not limited to, by introducing one or more than one nucleic acid comprising a gene fusion into a plant by transformation, or by introducing one, two, or more than two, silencing nucleic acid sequences, each silencing nucleic acid sequence comprising a sequence directed against a target gene, into a plant by transformation. Alternatively, silencing nucleic acid sequences may be introduced into a plant by crossing a first plant with a second plant that comprises one or more than one first gene fusion, or by crossing a first plant comprising one or more than one first gene fusion with a second plant comprising one or more than one second gene fusion. Silencing nucleic acid sequences may also be introduced into a plant by crossing a first plant with a second plant that comprises one, two, or more than two, silencing nucleic acid sequences, each silencing nucleic acid sequence comprising a sequence directed at silencing a target gene, or by crossing a first plant comprising one, two, or more than two, silencing nucleic acid sequences, each silencing nucleic acid sequence comprising a sequence directed against a target gene with a second plant comprising one, two, or more than two, silencing nucleic acid sequences, each silencing nucleic acid sequence comprising a sequence directed against a target gene.

The anti-nutrient factor may be sinapine, and the one, or two or more than two, enzymes may be involved in the pathway of phenylpropanoid biosynthesis. Non-limiting examples of the one, or two or more than two, enzymes include:

phenylalanine ammonia lyase (PAL),

cinnamate 4 hydroxylase (C4H),

coumarate 3 hydroxylase (C3H),

O-methyl transferase (OMT; or COMT),

ferulic acid hydroxylase (FAH),

sinapate:UDP-glucose sinapoyltransferase (SGT),

sinapolyglucose:choline sinapoyltransferase (SCT),

S-adenosylmethionine synthase (SAMS),

SAMS catalyses the synthesis of the methyl donor SAM (S-adenosyl methionine) from methionine and affects methylation reactions catalyzed by COMT (or OMT) in the phenylpropanoid pathway.

Therefore, a silencing nucleotide sequence may be directed to one, two, three, four, five, six, seven or eight genes listed above, alone or in combination. As stated above, a silencing nucleotide sequence may comprise a gene fusion that comprises two or more that two nucleotide sequence that disrupts the synthesis of two or more than two enzymes involved in phenylpropanoid, phytate biosynthesis or both, for example, sinapine and phytate synthesis, sinapine and lignin biosynthesis, lignin and phytate biosynthesis, or sinapine, lignin and phytate biosynthesis.

The anti-nutrient factor may also be lignin, and the one, two or more than two enzymes, involved in a pathway of phenylpropanoid biosynthesis. Non-limiting examples of the one, or two or more than two, enzymes include

phenylalanine ammonia lyase (PAL),

cinnamic acid 4 hydroxylase (C4H),

coumaric acid 3 hydroxylase (C3H),

caffeic acid O-methyl transferase (COMT),

ferulic acid hydroxylase (FAH),

S-adenosylmethionine synthase (SAMS),

4-coumarate:CoA lyase (4CL),

cinnamoyl-CoA reductase (CCR),

cinnamyl alcohol dehydrogenase (CAD), and

peroxidase (POD)

Therefore, a silencing nucleotide sequence may be directed to one, two, three, four, five, six, seven or eight genes listed above, either alone or in combination. Furthermore, a silencing nucleotide sequence may comprise a gene fusion that comprises two or more that two nucleotide sequence that disrupts the synthesis of two or more than two enzymes involved in phenylpropanoid, phytate biosynthesis or both, for example, sinapine and phytate synthesis, sinapine and lignin biosynthesis, lignin and phytate biosynthesis, or sinapine, lignin and phytate biosynthesis.

The anti-nutrient factor may also be phytate, and the one, two or more than two enzymes may be involved in a pathway of phytate biosynthesis. Non-limiting examples of the one, or two or more than two, enzymes include:

inositol 1,3,4-trisphosphate 5/6-kinase (IP3K),

myo-inositol hexaphosphate kinase (IP6K),

1-phosphatidylinositol-4,5-bisphosphate phosphodiesterase (PIBP PDE),

phosphatidylinositol phophatidylcholine transfer protein (PI/PC TP),

inositol polyphosphate 5-phosphatase II (IPP),

phosphatidylinositol-4-phosphate 5-kinase (PIKa),

CDP-diacylglycerol-inositol 3-phosphatidyltransferase (phosphatidylinositol synthase, PIS),

inositol polyphosphate 6-/3-/5-kinase 2b (IPK2a & IPK2b),

inositol polyphosphate 5′-phosphatase I (IPP),

myo-inositol-1-phosphate synthase (MIP),

phosphatidylinositol kinase (PIPK),

phosphatidylinositol 3-kinase (PI3K),

myo-inositol monophosphatase (MIM), and

phosphatidylinositol 3- and 4-kinase (PIKb).

Therefore, a silencing nucleotide sequence may be directed to one, two, three, four, five, six, seven or eight genes listed above, either alone or in combination. Furthermore, a silencing nucleotide sequence may comprise a gene fusion that comprises two or more that two nucleotide sequence that disrupts the synthesis of two or more than two enzymes involved in phenylpropanoid, phytate biosynthesis or both, for example, sinapine and phytate synthesis, sinapine and lignin biosynthesis, lignin and phytate biosynthesis, or sinapine, lignin and phytate biosynthesis.

Furthermore, analogues of any of the silencing nucleotide sequences encoding the above proteins may be used according to the present invention. An “analogue” or “derivative” includes any substitution, deletion, or addition to the silencing nucleotide sequence, provided that the nucleotide sequence retains the property of silencing expression of a target gene or sequence, reducing expression of a target sequence, or reducing synthesis or activity of a protein encoded by the target sequence. For example, derivatives, and analogues of nucleic acid sequences typically exhibit greater than 80% similarity with, a silencing nucleic acid sequence. Sequence similarity, may be determined by use of the BLAST algorithm (GenBank: www.ncbi.nlm.nih.gov/cgi-bin/BLAST/), using default parameters (Program: blastn; Database: nr; Expect 10; filter: low complexity; Alignment: pairwise; Word size:11). Analogs, or derivatives thereof, also include those nucleotide sequences that hybridize under stringent hybridization conditions (see Maniatis et al., in Molecular Cloning (A Laboratory Manual), Cold Spring Harbor Laboratory, 1982, p. 387-389) to any one of the sequences described herein, provided that the sequences exhibit the property of silencing expression of a target gene. An example of one such stringent hybridization conditions may be hybridization with a suitable probe, for example but not limited to, a [∀-³²P]dATP labelled probe for 16-20 hrs at 65EC in 7% SDS, 1 mM EDTA, 0.5M Na₂HPO₄, pH 7.2. Followed by washing in 5% SDS, 1 mM EDTA 40 mM Na₂HPO₄, pH 7.2 for 30 min followed by washing in 1% SDS, 1 mM EDTA 40 mM Na₂HPO₄, pH 7.2 for 30 min. Washing in this buffer may be repeated to reduce background.

By “regulatory region” “regulatory element” or “promoter” it is meant a portion of nucleic acid typically, but not always, upstream of the protein coding region of a gene, which may be comprised of either DNA or RNA, or both DNA and RNA. When a regulatory region is active, and in operative association, or operatively linked, with a gene of interest, this may result in expression of the gene of interest. A regulatory element may be capable of mediating organ specificity, or controlling developmental or temporal gene activation. A “regulatory region” includes promoter elements, core promoter elements exhibiting a basal promoter activity, elements that are inducible in response to an external stimulus, elements that mediate promoter activity such as negative regulatory elements or transcriptional enhancers. “Regulatory region”, as used herein, also includes elements that are active following transcription, for example, regulatory elements that modulate gene expression such as translational and transcriptional enhancers, translational and transcriptional repressors, upstream activating sequences, and mRNA instability determinants. Several of these latter elements may be located proximal to the coding region.

In the context of this disclosure, the term “regulatory element” or “regulatory region” typically refers to a sequence of DNA, usually, but not always, upstream (5′) to the coding sequence of a structural gene, which controls the expression of the coding region by providing the recognition for RNA polymerase and/or other factors required for transcription to start at a particular site. However, it is to be understood that other nucleotide sequences, located within introns, or 3′ of the sequence may also contribute to the regulation of expression of a coding region of interest. An example of a regulatory element that provides for the recognition for RNA polymerase or other transcriptional factors to ensure initiation at a particular site is a promoter element. Most, but not all, eukaryotic promoter elements contain a TATA box, a conserved nucleic acid sequence comprised of adenosine and thymidine nucleotide base pairs usually situated approximately 25 base pairs upstream of a transcriptional start site. A promoter element comprises a basal promoter element, responsible for the initiation of transcription, as well as other regulatory elements (as listed above) that modify gene expression.

There are several types of regulatory regions, including those that are developmentally regulated, inducible or constitutive. A regulatory region that is developmentally regulated, or controls the differential expression of a gene under its control, is activated within certain organs or tissues of an organ at specific times during the development of that organ or tissue. However, some regulatory regions that are developmentally regulated may preferentially be active within certain organs or tissues at specific developmental stages, they may also be active in a developmentally regulated manner, or at a basal level in other organs or tissues within the plant as well. Examples of tissue-specific regulatory regions, for example see-specific a regulatory region, include the napin promoter, and the cruciferin promoter (Rask et al., 1998, J. Plant Physiol. 152: 595-599; Bilodeau et al., 1994, Plant Cell 14: 125-130).

An inducible regulatory region is one that is capable of directly or indirectly activating transcription of one or more DNA sequences or genes in response to an inducer. In the absence of an inducer the DNA sequences or genes will not be transcribed. Typically the protein factor that binds specifically to an inducible regulatory region to activate transcription may be present in an inactive form, which is then directly or indirectly converted to the active form by the inducer. However, the protein factor may also be absent. The inducer can be a chemical agent such as a protein, metabolite, growth regulator, herbicide or phenolic compound or a physiological stress imposed directly by heat, cold, salt, or toxic elements or indirectly through the action of a pathogen or disease agent such as a virus. A plant cell containing an inducible regulatory region may be exposed to an inducer by externally applying the inducer to the cell or plant such as by spraying, watering, heating or similar methods. Inducible regulatory elements may be derived from either plant or non-plant genes (e.g. Gatz, C. and Lenk, I. R. P., 1998, Trends Plant Sci. 3, 352-358; which is incorporated by reference). Examples, of potential inducible promoters include, but not limited to, tetracycline-inducible promoter (Gatz, C., 1997, Ann. Rev. Plant Physiol. Plant Mol. Biol. 48, 89-108; which is incorporated by reference), steroid inducible promoter (Aoyama, T. and Chua, N. H., 1997, Plant J. 2, 397-404; which is incorporated by reference) and ethanol-inducible promoter (Salter, M. G., et al, 1998, Plant Journal 16, 127-132; Caddick, M. X., et al,1998, Nature Biotech. 16, 177-180, which are incorporated by reference) cytokinin inducible IB6 and CKI1 genes (Brandstatter, I. and Kieber, J. J., 1998, Plant Cell 10, 1009-1019; Kakimoto, T., 1996, Science 274, 982-985; which are incorporated by reference) and the auxin inducible element, DR5 (Ulmasov, T., et al., 1997, Plant Cell 9, 1963-1971; which is incorporated by reference).

A constitutive regulatory region directs the expression of a gene throughout the various parts of a plant and continuously throughout plant development. Examples of known constitutive regulatory elements include promoters associated with the CaMV 35S transcript. (Odell et al., 1985, Nature, 313: 810-812), the rice actin 1 (Zhang et al, 1991, Plant Cell, 3: 1155-1165), actin 2 (An et al., 1996, Plant J., 10: 107-121), or tms 2 (U.S. Pat. No. 5,428,147, which is incorporated herein by reference), and triosephosphate isomerase 1 (Xu et. al., 1994, Plant Physiol. 106: 459-467) genes, the maize ubiquitin 1 gene (Cornejo et al, 1993, Plant Mol. Biol. 29: 637-646), the Arabidopsis ubiquitin 1 and 6 genes (Holtorf et al, 1995, Plant Mol. Biol. 29: 637-646), and the tobacco translational initiation factor 4A gene (Mandel et al, 1995 Plant Mol. Biol. 29: 995-1004). The term “constitutive” as used herein does not necessarily indicate that a gene under control of the constitutive regulatory region is expressed at the same level in all cell types, but that the gene is expressed in a wide range of cell types even though variation in abundance is often observed.

The silencing nucleotide sequence may be expressed in any suitable plant host that is transformed by the nucleotide sequence, or constructs, or vectors of the present invention. Examples of suitable hosts include, but are not limited to, agricultural crops including canola, Brassica spp., maize, tobacco, alfalfa, potato, ginseng, pea, oat, rice, soybean, wheat, barley, sunflower, and cotton. Any member of the Brassica-family can be transformed with one or more genetic constructs of the present invention including, but not limited to, canola, Brassica napus, B. carinata, B. nigra, B. oleracea, B. chinensis, B. cretica, B. incana, B. insularis, B. japonica, B. atlantica, B. bourgeaui, B. narinosa, B. juncea, B. rapa, Arabidopsis. Additionally, woody plants and trees may be transformed with a silencing nucleotide sequence or construct of the present invention to reduce fiber content.

The one or more chimeric genetic constructs of the present invention can further comprise a 3′ untranslated region. A 3′ untranslated region refers to that portion of a gene comprising a DNA segment that contains a polyadenylation signal and any other regulatory signals capable of effecting mRNA processing or gene expression. The polyadenylation signal is usually characterized by effecting the addition of polyadenylic acid tracks to the 3′ end of the mRNA precursor. Polyadenylation signals are commonly recognized by the presence of homology to the canonical form 5′ AATAAA-3′ although variations are not uncommon. One or more of the chimeric genetic constructs of the present invention can also include further enhancers, either translation or transcription enhancers, as may be required. These enhancer regions are well known to persons skilled in the art, and can include the ATG initiation codon and adjacent sequences. The initiation codon must be in phase with the reading frame of the coding sequence to ensure translation of the entire sequence.

Non-limiting examples of suitable 3′ regions are the 3′ transcribed non-translated regions containing a polyadenylation signal of Agrobacterium tumor inducing (Ti) plasmid genes, such as the nopaline synthase (Nos gene) and plant genes such as the soybean storage protein genes and the small subunit of the ribulose-1,5-bisphosphate carboxylase (ssRUBISCO) gene.

To aid in identification of transformed plant cells, the constructs of this invention may be further manipulated to include plant selectable markers. Useful selectable markers include enzymes that provide for resistance to chemicals such as an antibiotic for example, gentamycin, hygromycin, kanamycin, or herbicides such as phosphinothrycin, glyphosate, chlorosulfuron, and the like. Similarly, enzymes providing for production of a compound identifiable by colour change such as GUS (beta-glucuronidase), or luminescence, such as luciferase or GFP, may be used.

Also considered part of this invention are transgenic plants containing the chimeric gene construct of the present invention. Methods of regenerating whole plants from plant cells are also known in the art. In general, transformed plant cells are cultured in an appropriate medium, which may contain selective agents such as antibiotics, where selectable markers are used to facilitate identification of transformed plant cells. Once callus forms, shoot formation can be encouraged by employing the appropriate plant hormones in accordance with known methods and the shoots transferred to rooting medium for regeneration of plants. The plants may then be used to establish repetitive generations, either from seeds or using vegetative propagation techniques. Transgenic plants can also be generated without using tissue cultures.

The constructs of the present invention can be introduced into plant cells using Ti plasmids, Ri plasmids, plant virus vectors, direct DNA transformation, micro-injection, electroporation, etc. For reviews of such techniques see for example Weissbach and Weissbach, Methods for Plant Molecular Biology, Academy Press, New York VIII, pp. 421-463 (1988); Geierson and Corey, Plant Molecular Biology, 2d Ed. (1988); and Miki and Iyer, Fundamentals of Gene Transfer in Plants. In Plant Metabolism, 2d Ed. D T. Dennis, D H Turpin, D D Lefebrve, D B Layzell (eds), Addison Wesly, Langmans Ltd. London, pp. 561-579 (1997); Clough and Bent (1998)).

Reducing Anti-Nutrient Synthesis

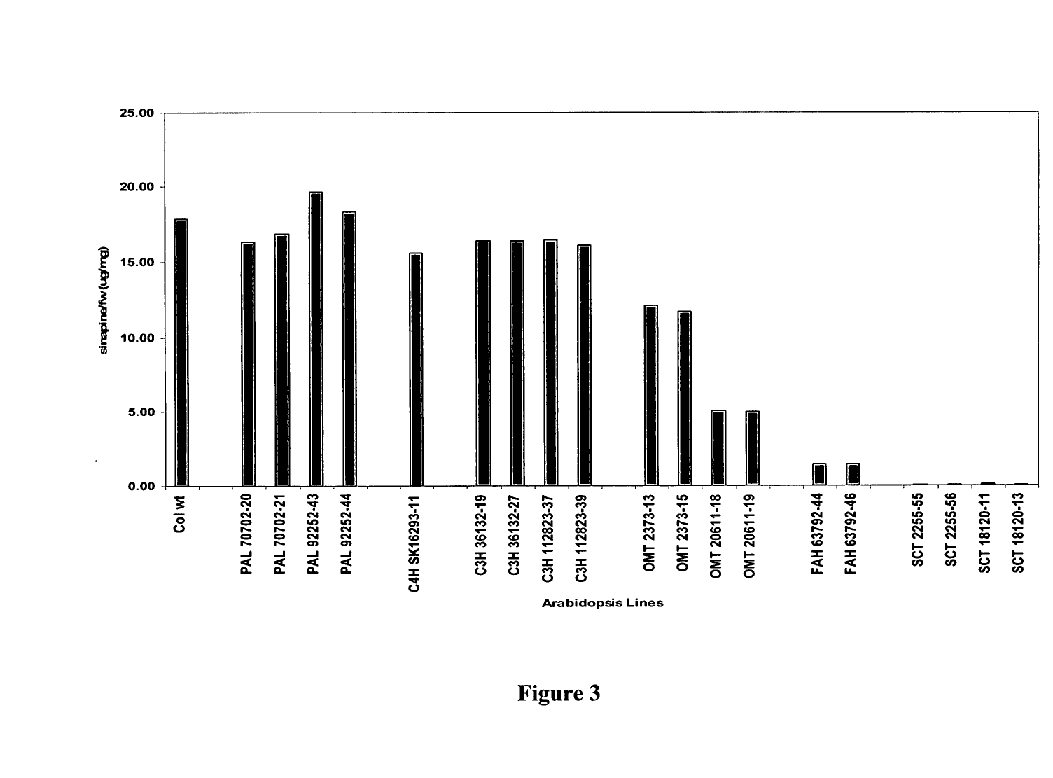

Arabidopsis knockout mutants within the phenylpropanoid pathway were identified, and seeds obtained from the mutants analyzed (see FIG. 1, and examples for details). Knockouts in genes in the early steps in the pathway had negligible impact on sinapine accumulation. However, knockouts in genes affecting latter steps resulted in up to increased reduction in sinapine content (FIG. 3; e.g. caffeic acid O-methyltransferase (COMT), ferulic acid hydroxylase (FAH), and sinapolyglucose:choline sinapoyltransferase (SCT)).

Homologs of genes encoding several enzymes involved in the phenylpropanoid pathway and the synthesis of sinapine were obtained and used to generate silencing nucleotide sequences (RNAi and antisense constructs). Expression of the silencing nucleotide sequences in seeds of B. napus, resulted in reduced levels of anti-nutrient factors, for example sinapine (see Example 1), in the seed. For example:

B. napus lines expressing an RNAi construct specific SCT (CaMV35S::SCT RNAi, p72-145, FIG. 4a) had up to a 51% reduction in sinapine when compared to a wild type B. napus (see FIG. 4b);

B. napus lines expressing an RNAi construct specific COMT (CaMV35S::COMT RNAi, p72-115, FIG. 14a) had up to a 17% reduction in sinapine when compared to a wild type B. napus (see FIG. 14b);

an antisense construct expressing the gene fusion FAH-SCT (napin::FAH-SCT, p72-148, FIG. 5a;) resulted in reductions of up to 90%, compared to the wild type (FIGS. 5c and 5d);

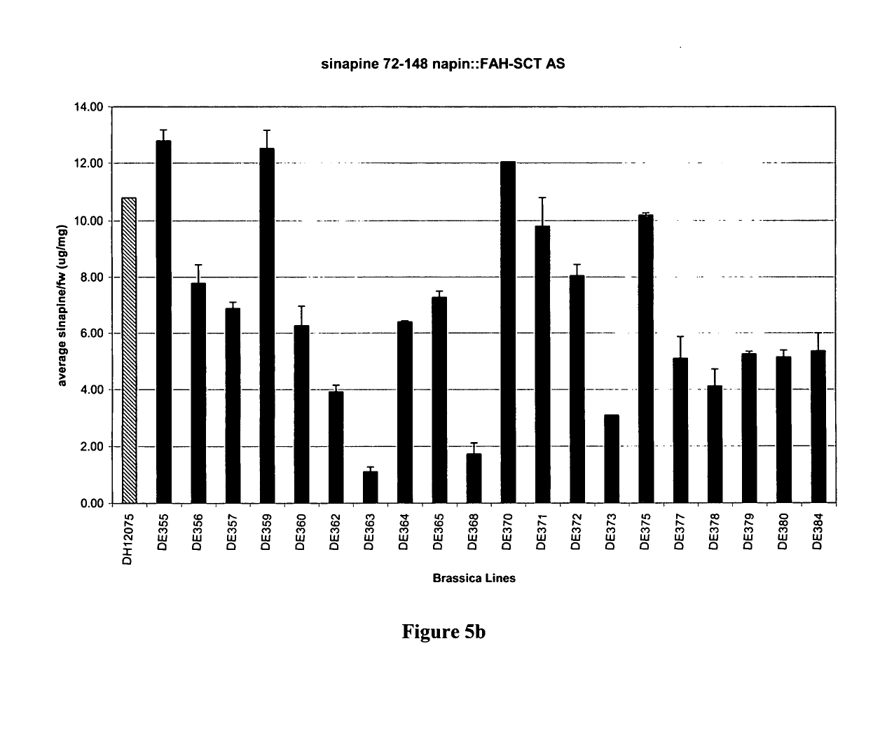

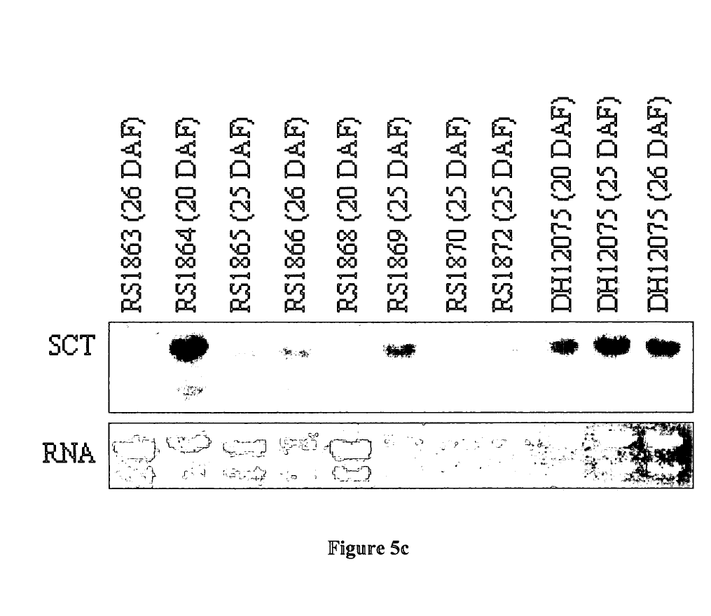

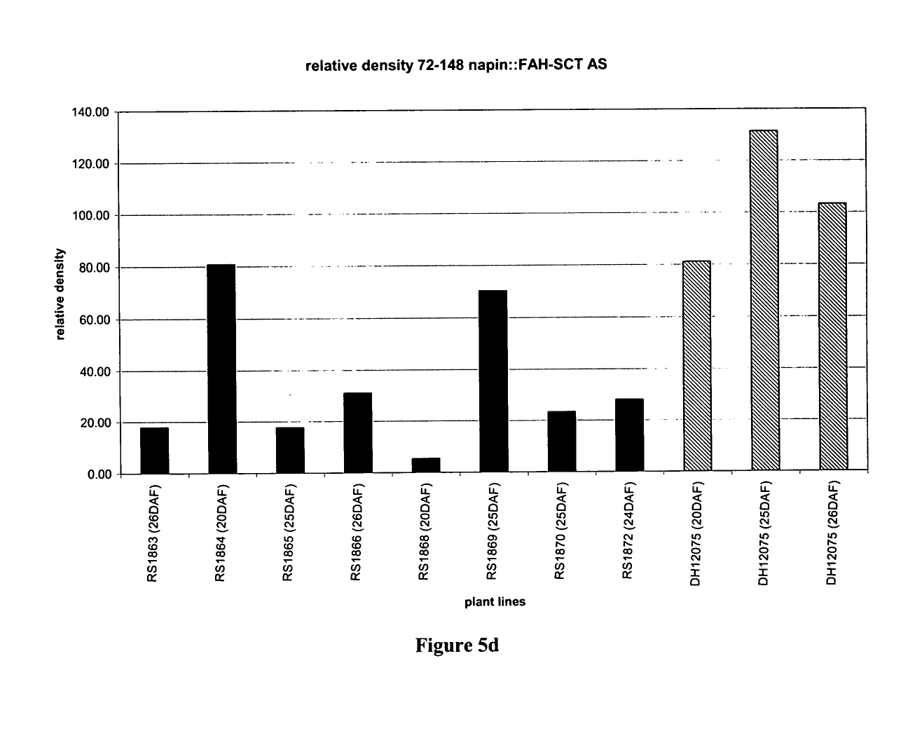

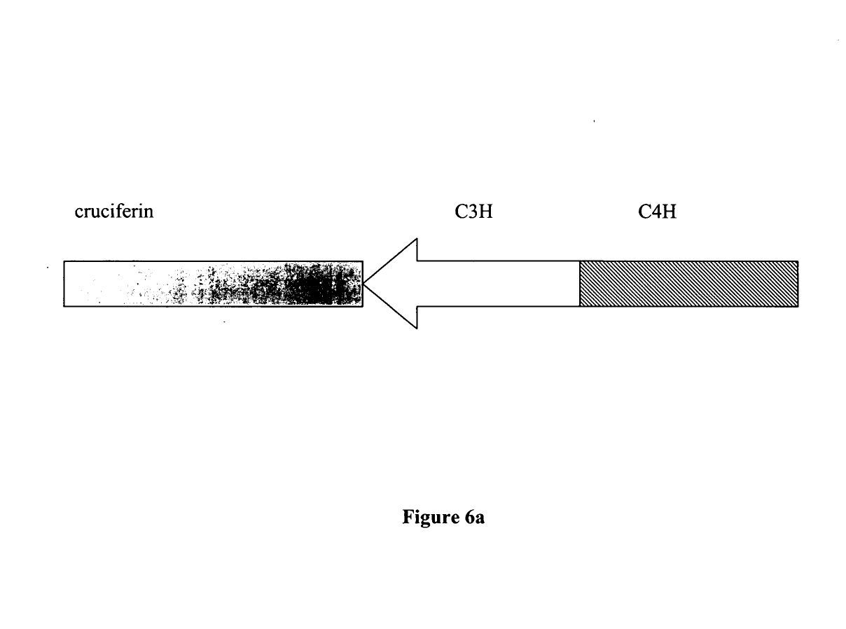

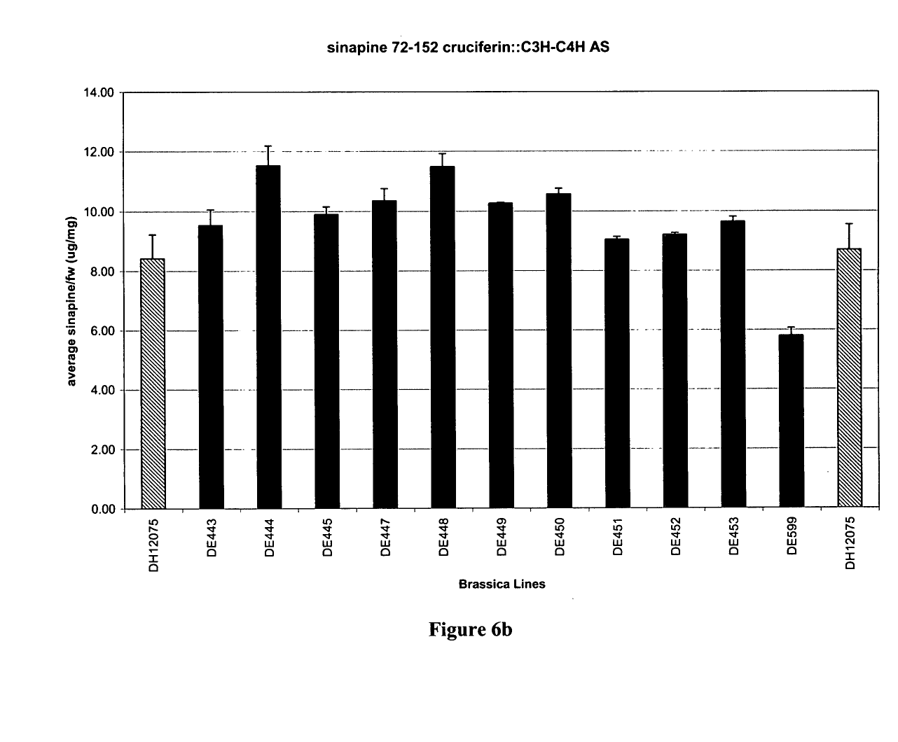

an antisense gene fusion, C3H-C4H, under the control of the seed-specific promoter cruciferin (Cruciferin::C3H-C4H, p72-152, FIG. 6a) resulted in 33% reduction in seed sinapine compared to the wild type line (FIG. 6b);

an RNAi gene fusion construct, C3H-C4H, under the control of the constitutive promoter CaMV35S (CaMV35S::C3H-C4H RNAi, p72-146, FIG. 7a) resulted in a reduction of sinapine of 36% (FIG. 7b) when compared to sinapine levels in seeds from the control plant;

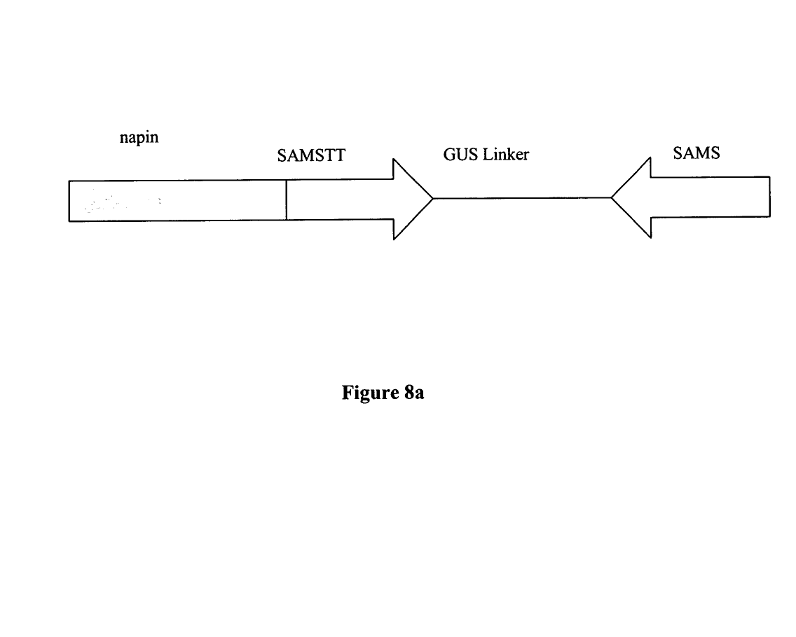

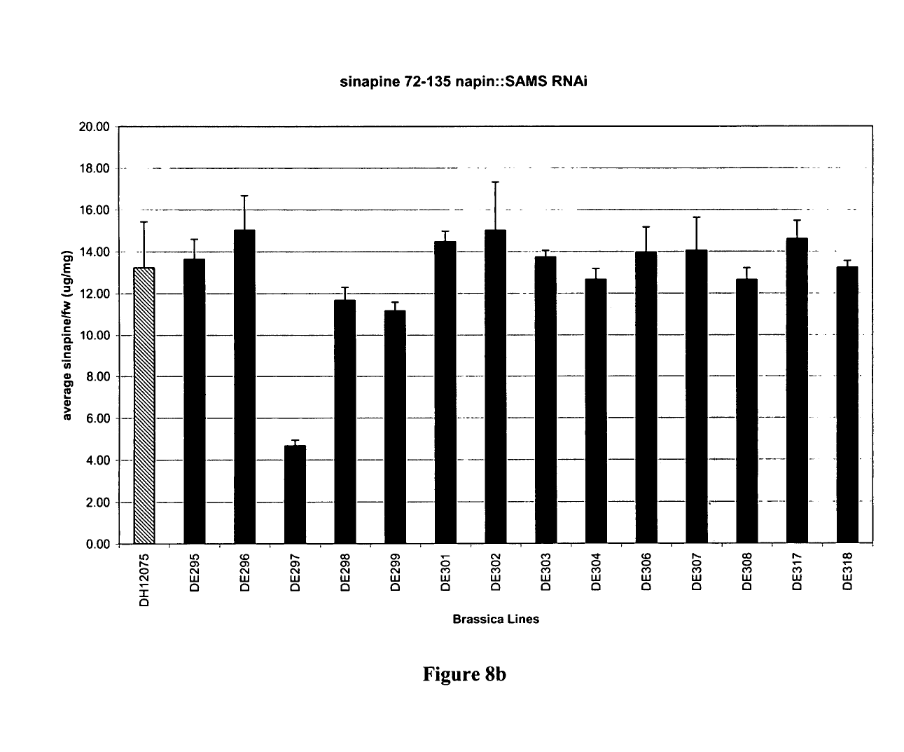

an RNAi construct directed against SAMS, under the control of the seed-specific promoter napin caused (Napin::SAMS RNAi, p72-135, FIG. 8a) resulted in a reduction of sinapine of 65% when compared to sinapine levels in seeds from the wild type line (FIG. 8b);

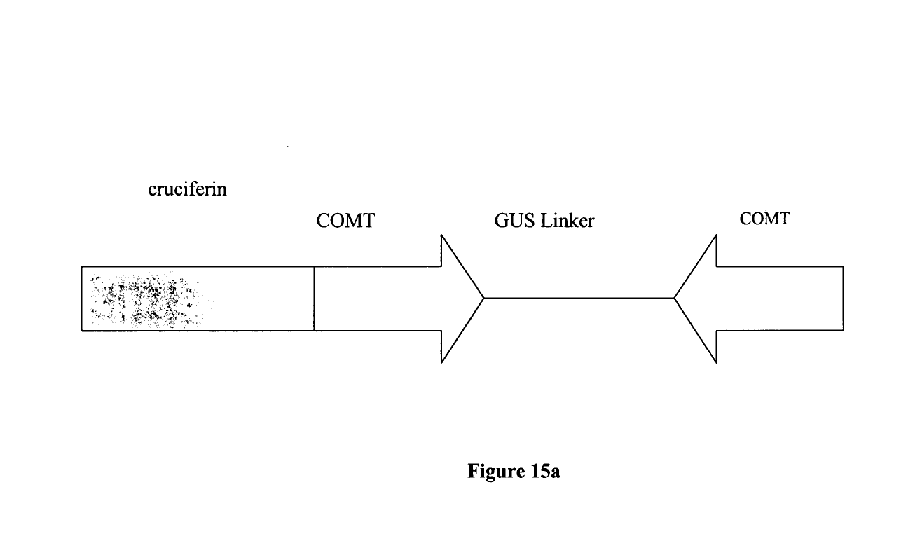

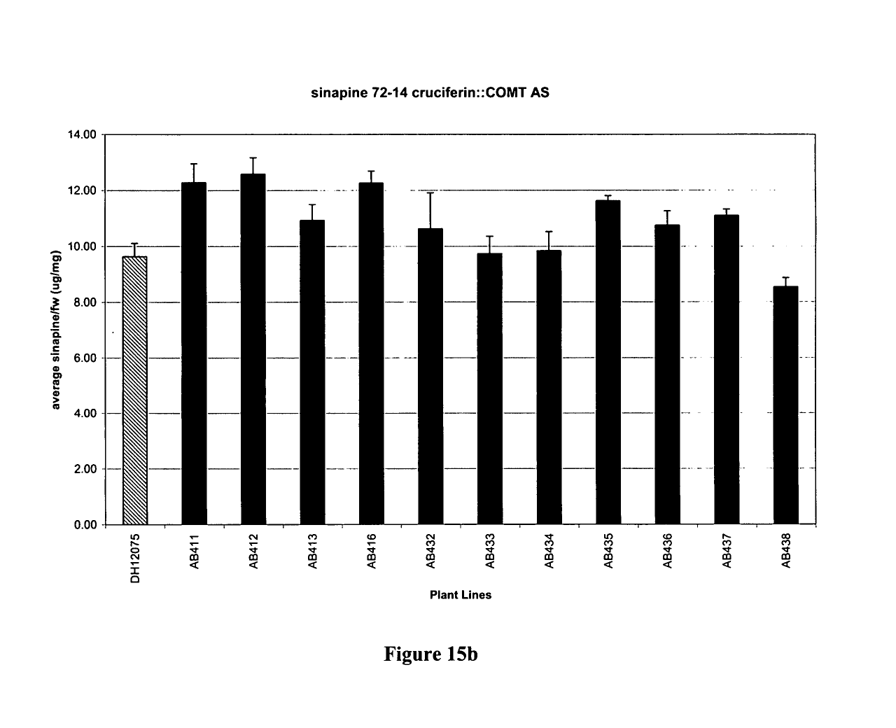

an antisense construct directed against COMT (cruciferin::COMT, p72-14, FIG. 15a) under the control of the seed-specific promoter cruciferin resulted in reductions of up to 11.4%, compared to the wild type (FIG. 15b);

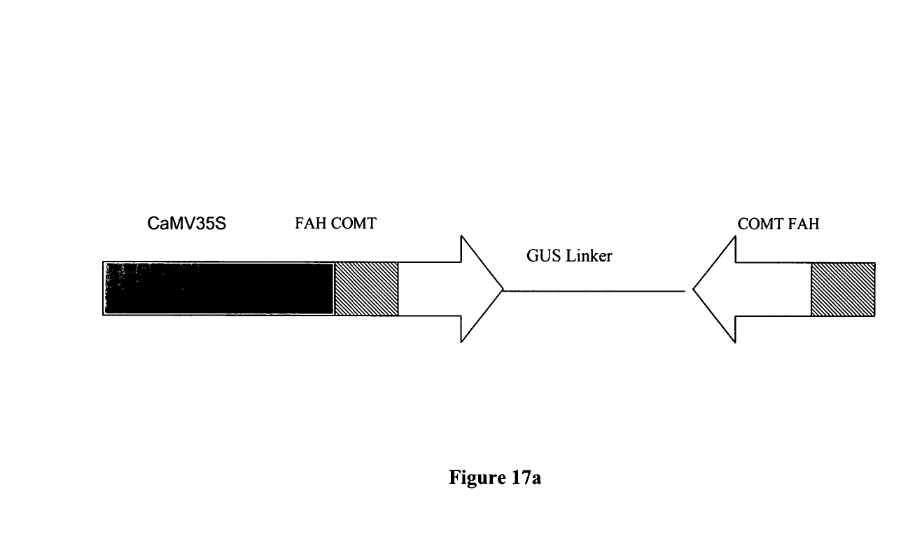

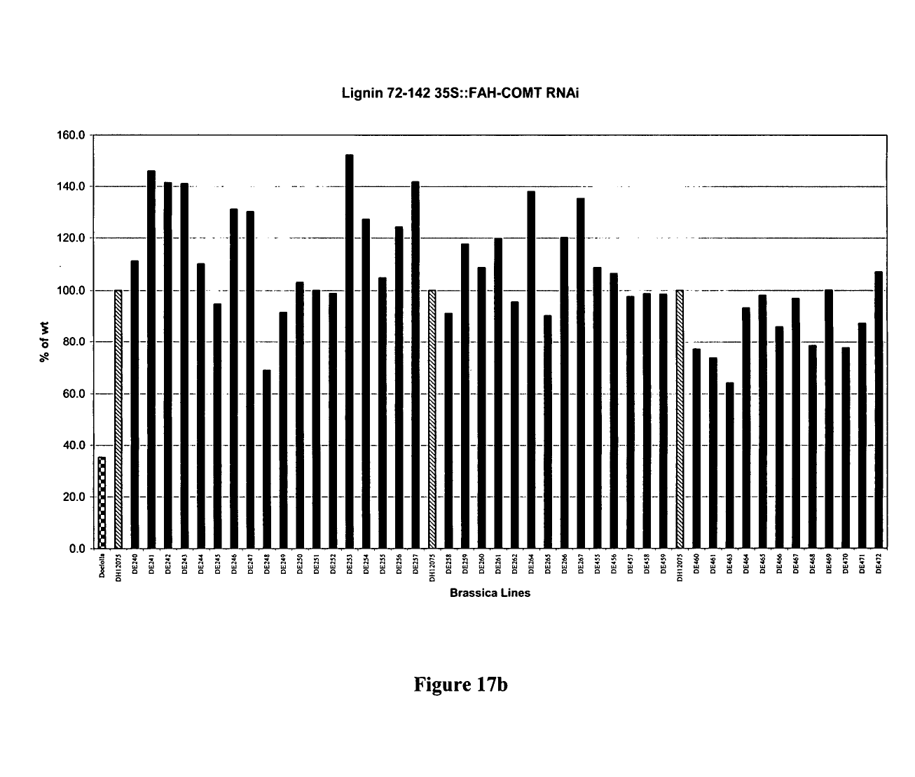

an RNAi gene fusion construct, FAH-COMT, under the control of the constitutive promoter CaMV35S (CaMV35S:: FAH-COMT RNAi, p72-142, FIG. 17a) resulted in a reduction of sinapine of 17.8% (FIG. 17b) when compared to sinapine levels in seeds from the control plant.

These results indicate that silencing nucleotide sequences may be used to reduce anti-nutrient factors synthesized within the phenylpropanoid pathway.

B. napus lines were also developed with silencing nucleotide sequences that interfered with the expression of genes encoding enzymes involved in lignin biosynthesis (see Example 2), including caffeic acid O-methyltransferase (COMT), ferulic acid hydroxylase (FAH), S-adenosylmethionine synthase (SAMS), cinnamic acid 4-hydroxylase (C4H) and coumaric acid 3-hydroxylase (C3H), 4-coumarate ligase (4CL), cinnamoyl CoA reductase (CCR).

Genes encoding several enzymes involved in the phenylpropanoid pathway and the synthesis of lignin were obtained and used to generate silencing nucleotide sequences (RNAi and antisense constructs). Expression of the silencing nucleotide sequences in seeds of B. napus, resulted in reduced levels of anti-nutrient factors, for example lignin, in the seed For example:

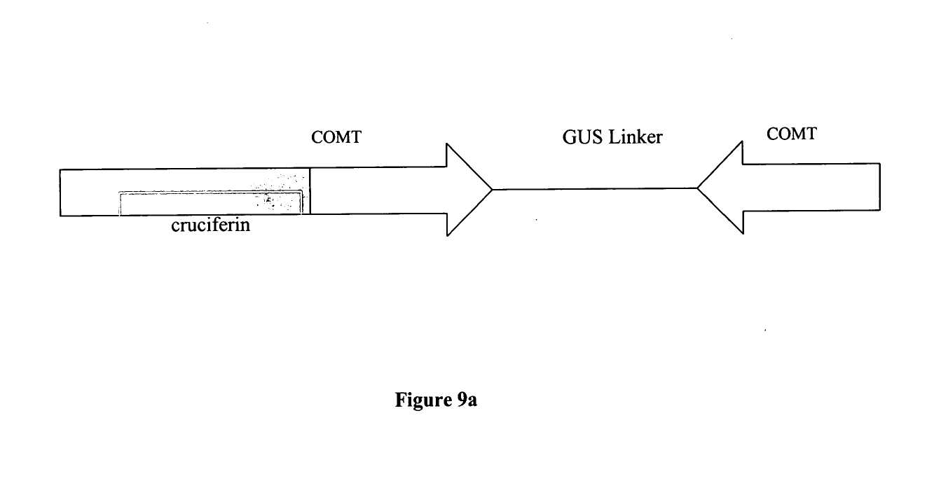

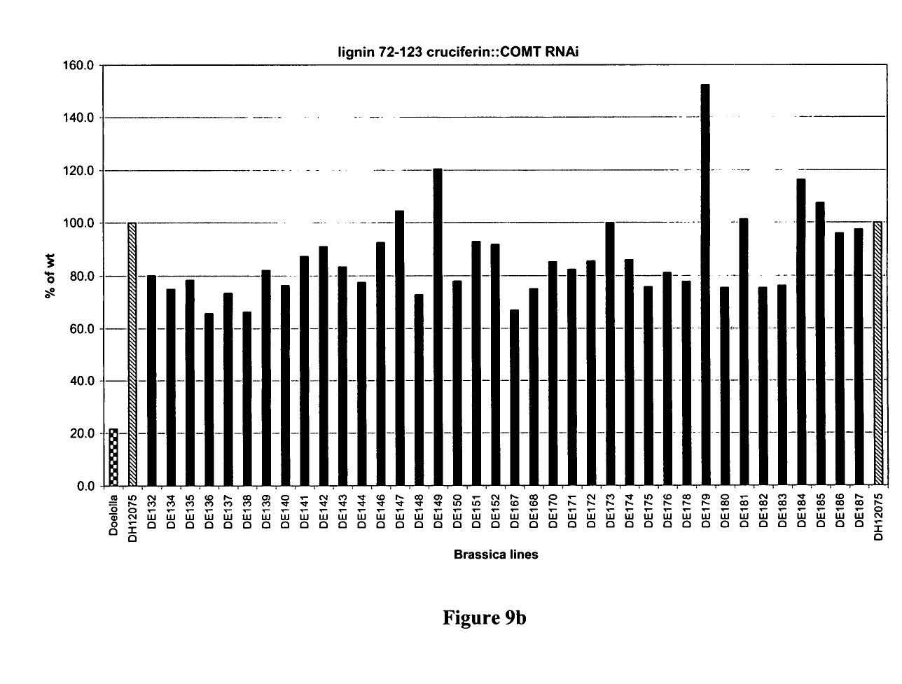

expression of a COMT RNAi construct under control of a cruciferin promoter (Cruciferin::COMT FIG. 9a, p72-123) caused a reduction in lignin levels of up to 34% (FIG. 9b) when compared to wild type lignin levels;

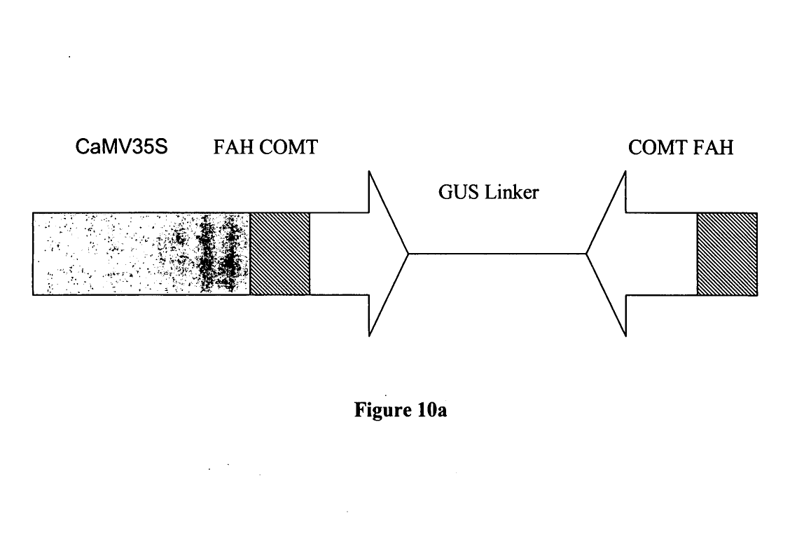

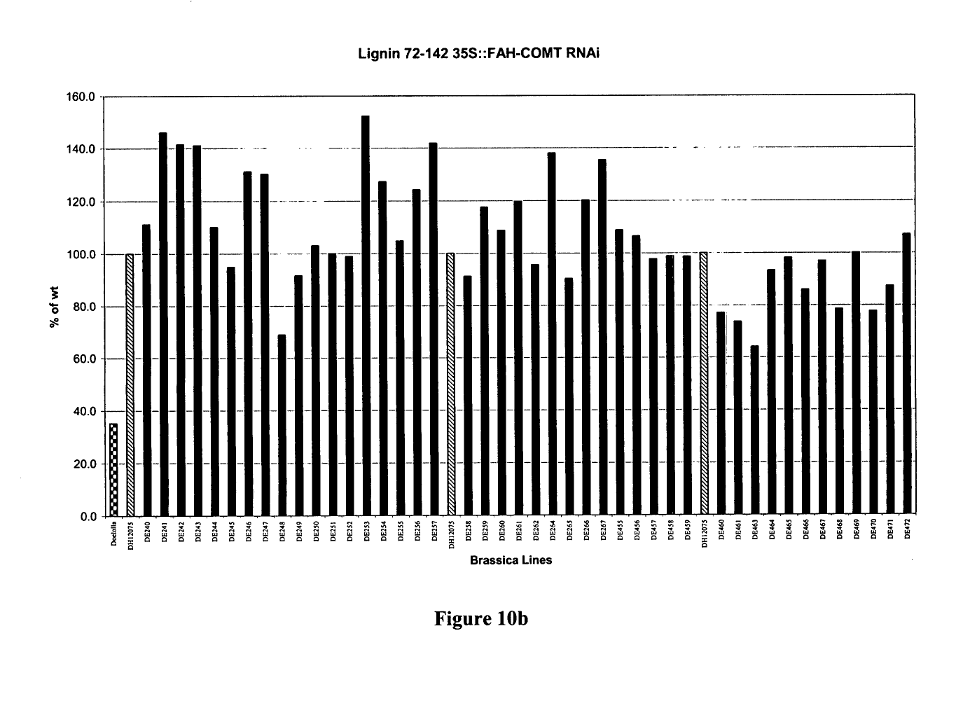

an RNAi gene fusion construct directed against FAH-COMT (FIG. 10a, p72-142) under the control of the constitutive promoter CaMV35S resulted in a reduction in seed lignin content of up to 36% relative to the wild type (FIG. 10b);

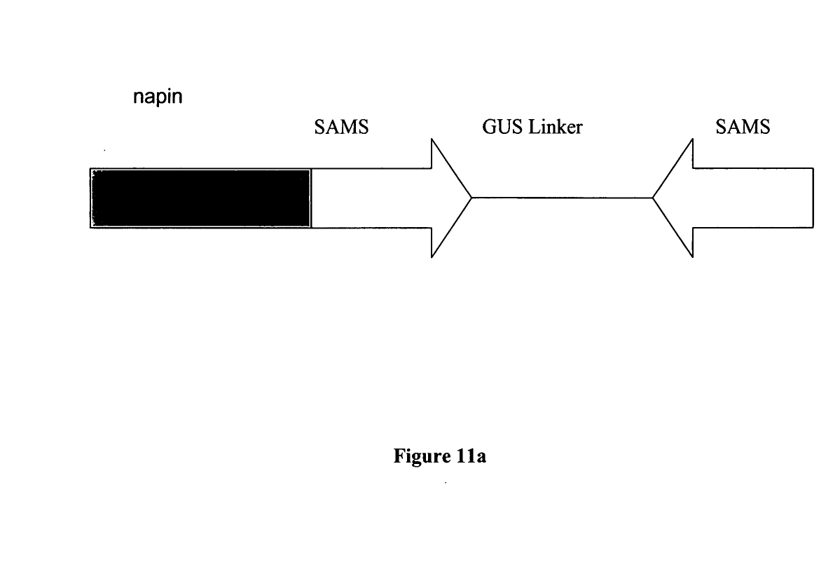

expression of SAMS RNAi construct under the control of a napin promoter (FIG. 11a, p72-135) caused a reduction in lignin of about 17% relative to the wild type (FIG. 11b).

B. napus expressing the antisense construct napin::COMT (FIG. 12a, p72-122) resulted in a reduction in lignin of up to 23% relative to the lignin levels in the wild type (FIG. 12b);

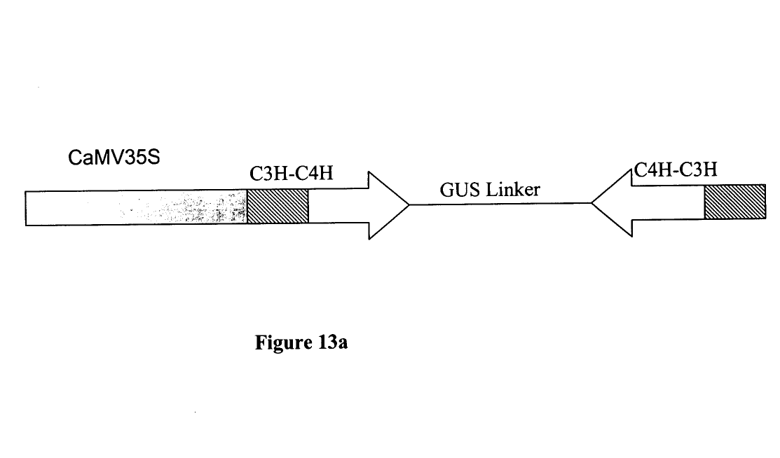

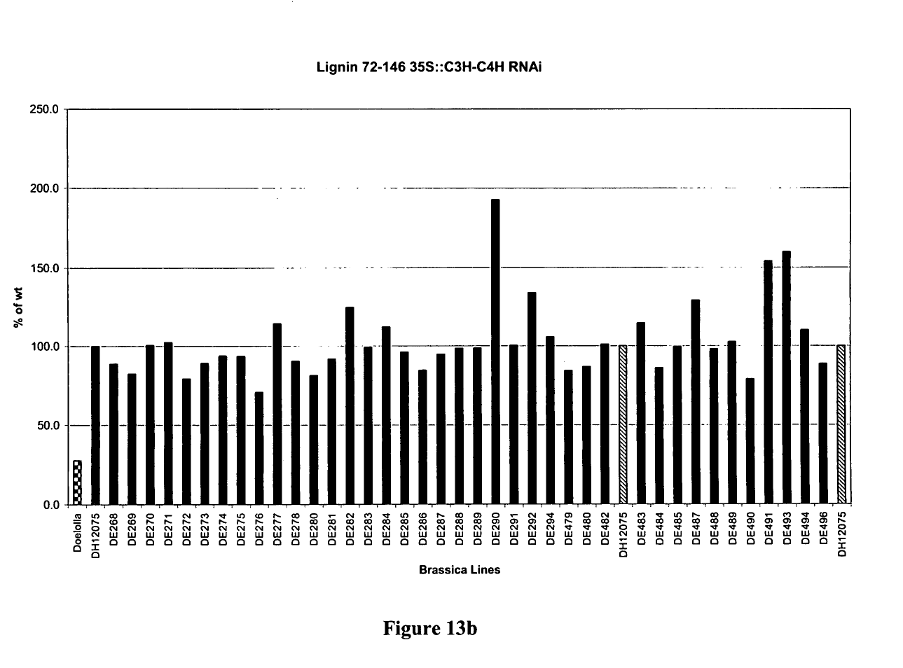

expressing the RNAi construct CaMV35S::C3H-C4H (FIG. 13a, p72-146) resulted in reduced seed lignin content of about 29% when compared to wild type (FIG. 13b).

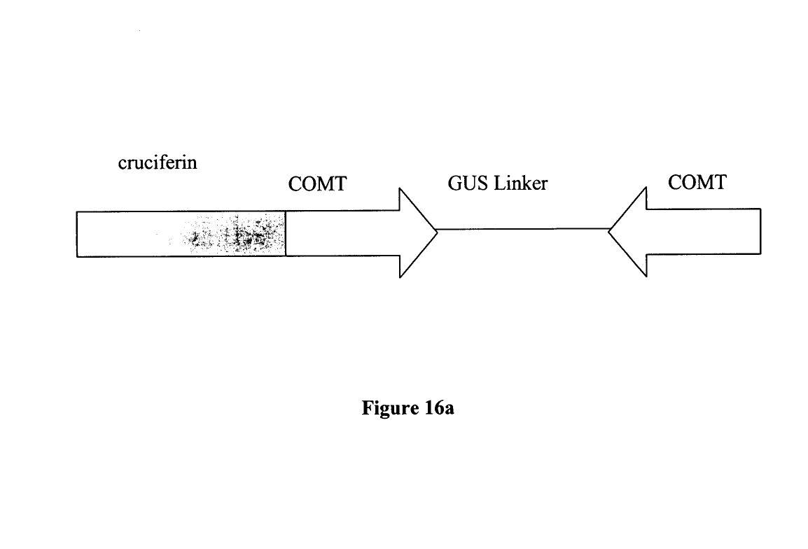

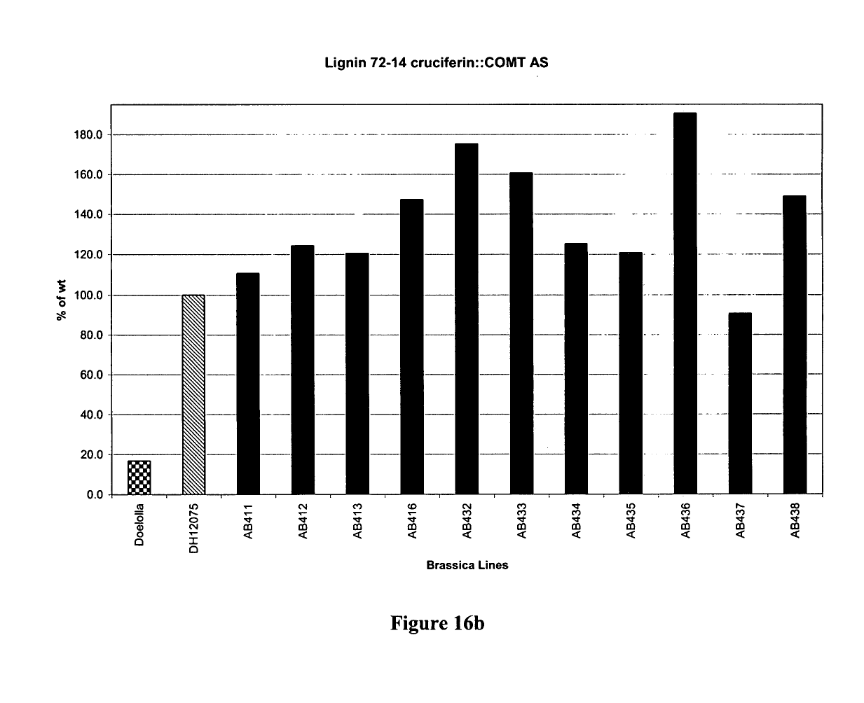

B. napus expressing the antisense construct cruciferin::COMT (FIG. 16a, p72-14) resulted in a reduction in lignin of up to 9.4% relative to the lignin levels in the wild type (FIG. 16b);

These results additionally indicate that silencing nucleotide sequences may be used to reduce anti-nutrient factors synthesized within the phenylpropanoid pathway.

B. napus lines were also developed with silencing nucleotide sequences that interfered with the expression of genes encoding enzymes involved in phytate biosynthesis (see FIG. 14, Example 3).

Genes encoding several enzymes involved in the pathway leading to the synthesis of phytate were obtained and used to generate silencing nucleotide sequences (RNAi and antisense constructs). Expression of the silencing nucleotide sequences in seeds of B. napus, resulted in reduced levels of anti-nutrient factors, for example phytate, in the seed (see Table 4, Example 3). For example:

levels of phytate B. napus seeds expressing the gene fusion RNAi construct IP3K-IP6K (Actin2::IP3K-IP6KP, p72-535), resulted in a 19% reduction in phytate levels when compared to wild type levels;

an RNAi gene fusion construct, PIKa-PIKb (CaMV35S::PIKₐ-PIK_{b}, p72-528) resulted in a reduction of seed phytate levels of about 13% when compared to wild type phytate levels.

B. napus plants expressing IP6K (Actin2::IP6K, p72-536) resulted in a reduction of seed phytate levels of about 16.5% when compared to wild type phytate levels;

the RNAi construct, PIKa under the control of an actin promoter (Actin2::PIKₐ, p72-537), resulted in a reduction of seed phytate levels of about 29.4% when compared to wild type phytate levels.

These results additionally show that silencing nucleotide sequences may be used to reduce anti-nutrient factors synthesized within the phytate biosynthetic pathway.

Most of the seed phytate is synthesized through the step-wise phosphorylation of myo-inositol rather than through phosphatidyl inositol. An alternate strategy to reduce phytate levels involves diverting more myo-inositol to phosphatidyl inositol biosynthesis, combined with blocking the de-esterification of the latter to myo-inositol phosphate. This is achieved by overexpressing phosphatidyl inositol synthase (PIS), by ectopically expressing PIS, and silencing phosphotidyl inositol bisphosphate phosphodiesterase (PIBP PDE), using a silencing nucleotide sequence and the methods disclosed within the present invention.

The present invention provides a construct comprising a silencing nucleotide sequence, the silencing nucleotide sequence encoding one, or two or more than two sequences that when expressed in a host reduces or inhibits the expression of one, or two or more that two enzymes involved in the synthesis of one or more than one protein leading to sinapine biosynthesis, lignin biosynthesis, phytate biosynthesis, or a combination thereof, the reduced level of the one or more than one protein determined by comparing the level of the protein in the host, or a tissue of the host, with a level of the protein in a second host, or the tissue from the second host, that does not express the nucleic acid sequence.

The silencing nucleotide sequence may comprise an RNAi construct, or an antisense construct directed against one gene, or two or more genes, for example the construct may comprise a gene fusion directed to 2, 3, 4, 5, 6, 7 or 8 different genes within one or more than one biosynthetic pathway.

The present invention also provides for a plant comprising a nucleic acid sequence comprising a regulatory region operatively associated with a silencing nucleotide sequence, the silencing nucleotide sequence encoding one, or two or more than two sequences that reduce or eliminates the expression of one, or two or more than two enzymes involved in the synthesis of the one or more than one protein leading to sinapine biosynthesis, lignin biosynthesis, phytate biosynthesis, or a combination thereof, the reduced level of the one or more than one protein determined by comparing the level of the protein in the plant, or a tissue of the plant, with a level of the protein in a second plant, or the tissue from the second plant, that does not express the nucleic acid sequence.

The silencing nucleotide sequence may comprise an RNAi construct, or an antisense construct directed against one gene, or two or more genes, for example the construct may comprise a gene fusion directed to 2, 3, 4, 5, 6, 7 or 8 different genes within one or more than one biosynthetic pathway.

The present invention also provides a seed that is characterized as having reduced levels of one or more than one anti-nutrient compounds. Examples of an anti-nutrient compound include intermediates within the phenylpropanoid pathway, or products of the phenylpropanoid pathway including sinapine and lignin, the phytate biosynthetic pathway, including phytate. The level of the anti-nutrient compound may be reduced by about 10% to about 100%, or any amount therebetween, when compared to the level of the same anti-nutrient compound obtained from a second plant that does not express a silencing nucleotide sequence. For example, the anti-nutrient compound may be reduced by from about 10% to about 60% or any amount therebetween, about 10% to about 50% or any amount therebetween, about 10% to about 40% or any amount therebetween, or from about 10% to about 30%, or any amount therebetween, or about 10% to about 20% or any amount therebetween.

The present invention also provides for a method for reducing the level of one or more than one protein within a plant or a tissue within the plant comprising, expressing a nucleotide sequence within the plant or a tissue within the plant, the nucleic acid sequence comprising a regulatory region operatively associated with a silencing nucleotide sequence, where expression of the silencing nucleotide sequence reduces or eliminates the expression of one, or two or more than two enzymes involved in the synthesis of the one or more than one protein leading to sinapine biosynthesis, lignin biosynthesis, phytate biosynthesis, or a combination thereof, the reduced level of the one or more than one protein determined by comparing the level of the protein in the plant, or a tissue of the plant, with a level of the protein in a second plant, or the tissue from the second plant, that does not express the nucleic acid sequence.

Examples of suitable plants that may be modified using the methods as described herein include, but are not limited to, agricultural crops including canola, Brassica spp., maize, tobacco, alfalfa, potato, ginseng, pea, oat, rice, soybean, wheat, barley, sunflower, cotton, and woody plants including trees. Any member of the Brassica-family can be transformed with one or more genetic constructs of the present invention including, but not limited to, Arabidopsis, Brassica amplexicaulis, Brassica atlantica, Brassica balearica, Brassica barrelieri, Brassica bourgeaui, Brassica carinata (Abyssinian mustard), Brassica chinensis, Brassica cretica, Brassica deflexa, Brassica erucastrum, Brassica hilarionis, Brassica incana, Brassica insularis, Brassica insularis subsp. insularis, Brassica juncea (Indian mustard), Brassica macrocarpa, Brassica maurorum, Brassica montana, Brassica napus (rape), Brassica napus var. napobrassica (Swedish turnip), Brassica napus var. napus (canola), Brassica narinosa, Brassica nigra (black mustard), Brassica oleracea, Brassica oleracea var. acephala (kale), Brassica oleracea var. alboglabra (Chinese kale), Brassica oleracea var. botrytis (cauliflower), Brassica oleracea var. capitata (cabbage), Brassica oleracea var. gemmifera (brussel sprouts), Brassica oleracea var. gongylodes (kohlrabi), Brassica oleracea var. italica (asparagus broccoli), Brassica oleracea var. medullosa (marrow-stem kale), Brassica oleracea var. oleracea, Brassica oleracea var. ramosa (branching bush kale), Brassica oxyrrhina, Brassica rapa (field mustard), Brassica rapa subsp. chinensis (bok-choy), Brassica rapa subsp. oleifera (biennial turnip rape), Brassica rapa subsp. pekinensis (Chinese cabbage), Brassica rapa subsp. rapa (turnip), Brassica rupestris, Brassica tournefortii, Brassica villosa.

The following sequences are included in the sequence listing:

| | |
|---|---|
| SEQ ID NO: 1 | ESS2134 COMT |
| SEQ ID NO: 2 | pGSA1285 |
| SEQ ID NO: 3 | RL1627 FAH |
| SEQ ID NO: 4 | P72-505 SCT |
| SEQ ID NO: 5 | p72-134 FAH-SCT |
| SEQ ID NO: 6 | Cruciferin promoter |
| SEQ ID NO: 7 | RL103 SAMS |
| SEQ ID NO: 8 | Napin promoter |
| SEQ ID NO: 9 | RL4992 C3H |
| SEQ ID NO: 10 | RL118F C4H |
| SEQ ID NO: 11 | P72-139 C3h-C4h |
| | fusion |
| SEQ ID NO: 12 | RL4637 COMT |
| SEQ ID NO: 13 | PGSA1252 |
| SEQ ID NO: 14 | RL4142 4CL |
| SEQ ID NO: 15 | RL2419 CCR |
| SEQ ID NO: 16 | p72-168 4CL-CCR |
| | fusion |
| SEQ ID NO: 17 | CL396R PIKa |
| SEQ ID NO: 18 | ESS1196F PIKb |
| SEQ ID NO: 19 | p72-514 PIKa-PIKb |
| | fusion |
| SEQ ID NO: 20 | P72-509 PIK |
| SEQ ID NO: 21 | Actin2 promoter |
| SEQ ID NO: 22 | P72-507 IP6K |
| SEQ ID NO: 23 | RL1344R IP3K |
| SEQ ID NO: 24 | P72-512 IP6K-IP3K |
| | fusion |
| SEQ ID NO: 25 | OMT-F5 primer |
| SEQ ID NO: 26 | OMT R6 primer |
| SEQ ID NO: 27 | SNG2-FI primer |
| SEQ ID NO: 28 | SNG2-R2 primer |
| SEQ ID NO: 29 | SCT-F7 primer |
| SEQ ID NO: 30 | SCT-R8 primer |
| SEQ ID NO: 31 | FAH-F7 primer |
| SEQ ID NO: 32 | FAH-R9 primer |
| SEQ ID NO: 33 | SCT-F11 primer |
| SEQ ID NO: 34 | SCT-R8 primer |
| SEQ ID NO: 35 | cruc-F3 primer |
| SEQ ID NO: 36 | cruc-R4 primer |
| SEQ ID NO: 37 | OMT-F1 5′ primer |
| SEQ ID NO: 38 | OMT-R2 5′ primer |
| SEQ ID NO: 39 | COMT-F1 primer |
| SEQ ID NO: 40 | COMT-R2 primer |
| SEQ ID NO: 41 | SAMS-F5 primer |
| SEQ ID NO: 42 | SAMS-R6 primer |
| SEQ ID NO: 43 | CCR-F8 primer |
| SEQ ID NO: 44 | FAH-R10 primer |
| SEQ ID NO: 45 | OMT1-F7 primer |
| SEQ ID NO: 46 | OMT-R6 primer |
| SEQ ID NO: 47 | C3H-F3 primer |
| SEQ ID NO: 48 | C3H-R4 primer |
| SEQ ID NO: 49 | C4H-F3 primer |
| SEQ ID NO: 50 | C4H-R4 primer |
| SEQ ID NO: 51 | 4CL-F5 primer |
| SEQ ID NO: 52 | 4CL-R6 primer |
| SEQ ID NO: 53 | CCR-F5 primer |
| SEQ ID NO: 54 | CCR-R6 primer |
| SEQ ID NO: 55 | CCR-R6 primer |
| SEQ ID NO: 56 | 4CL-R7 primer |
| SEQ ID NO: 57 | CCR-F8 primer |
| SEQ ID NO: 58 | PIK-F11 primer |
| SEQ ID NO: 59 | PIK-R20 primer |
| SEQ ID NO: 60 | PIK-F19 primer |
| SEQ ID NO: 61 | PIK-R16 primer |
| SEQ ID NO: 62 | act-F5 primer |
| SEQ ID NO: 63 | act-R6 primer |
| SEQ ID NO: 64 | PIK-R12 primer |
| SEQ ID NO: 65 | PIK-F5/IP6K-F5 |
| | primer |
| SEQ ID NO: 66 | IP6K-R6 primer |
| SEQ ID NO: 67 | IP6K-R11 primer |
| SEQ ID NO: 68 | IP3K-F12 primer |
| SEQ ID NO: 69 | IP3K-F12 primer |

The present invention will be further illustrated in the following examples.

### EXAMPLES

Plant Material

Wild type and mutant A. thaliana, ecotype Columbia, were grown in RediEarth (W.R. Grace & Co., Ajax, Canada) soil in pots covered with window screens at 25 C, 16 h light/8 h dark cycles. Wild type and transgenic Brassica napus DH12075 plants were grown in soil prepared according to the protocol described in Stringham (1971) under conditions similar to those of A. thaliana. Seeds were collected from dry siliques of mature plants of A. thaliana and B. napus.

Arabidopsis Knockout Mutants

Arabidopsis knockout mutants were identified by searching the SALK T-DNA knockout population in the TAIR database. Gene-specific sequences were used in the search. When no knockout mutants could be identified for specific gene, a T-DNA knockout population, generated at Agriculture and Agri-Food Canada (see: brassica.ca/index e.shtml) was screened for mutants of interest using a PCR-based method (Sussman et al., 2000).

Transformation of Brassica napus

B. napus transformation was carried out following the procedures that are described in Moloney et al. (1989; which is incorporated herein by reference). The presence of transgenes in potential transformants was determined by PCR using transgene-specific primers (one primer from the promoter and the other from the coding region) and total plant genomic DNA as template using standard methods known to one of skill in the art.

Estimation of Gene Copy Numbers in Arabidopsis

Gene copy numbers were estimated using the BioVis software (see: brassica.ca), and set to a homology threshold of 80%.

ESTs and Constructs

B. napus homologs of target Arabidopsis genes were obtained from a collection of 66,960 expressed sequence tags (ESTs) derived from B. napus line DH12075 cDNA libraries generated at Agriculture and Agri-Food Canada, Saskatoon Research Centre (see: brassica.ca/index e.shtml). Where no ESTs could be identified, B. napus sequences were isolated by PCR using primers specific to conserved Arabidopsis sequences and a B. napus cDNA library as template, using techniques known to one of skill in the art.

Northern Blot Hybridization

Northern blot analysis was carried out on total RNA extracted from plant seeds to determine the level of gene expression in the wild type and transgenic lines. Hybridization with [α-³²P]dCTP-labeled probes was carried out for 16-20 h at 65° C. in 7% SDS, 1 mM EDTA, 0.5 M Na₂HPO₄, pH 7.2. Membranes were washed once in a solution of 5% SDS, 1 mM EDTA, 40 mM Na₂HPO₄ (pH 7.2) for 30 min, followed by washing in 1% SDS, 1 mM EDTA, 40 mM Na₂HPO₄ (pH 7.2) for 30 min. The membranes were subjected to autoradiography using X-OMAT XAR5 film, and the intensity of bands measured using densitometer Quantity One Software (BioRad). The strength of the Northern blot bands was normalized by expressing it as a percentage of the density of the respective 28S rRNA band on the RNA gel.

Phenolics Extraction and Quantification

Seeds of Brassica napus and Arabidopsis thaliana were ground to a fine powder in liquid nitrogen using a pestle and a mortar. Approximately 50 mg of ground seeds were extracted with 15 volumes (w/v) of extraction buffer (50% methanol, 1.5% acetic acid), auto-vortexed for 5 minutes, stored at −80° C. for 2 hours followed by 5 min at room temperature, then auto-vortexed for 5 minutes, and centrifuged at 20 000 g for 5 minutes to pellet cellular debris. The supernatant was filtered through a 0.2 μm filter, transferred to HPLC autosampler vials and 3 μl were injected into the HPLC. HPLC was performed using a Waters Alliance 2695 instrument equipped with a Symmetry 5 μm C18 column (3 mm×150 mm). A 20 minute linear gradient was applied using a flow rate of 0.4 ml/min from 5% to 95% solvent B (methanol) in solvent A (0.05% TFA in water). The compounds were detected photometrically using a Waters 996 photodiode array detector set to 332 nm. Sinapine bisulfate (from Ian McGregor, Agriculture and Agri-Food Canada, Saskatoon), was used as a standard The percent reduction of sinapine was calculated by comparing values obtained from seeds of transgenic lines to those of the wild type. Total phenolic compounds absorbing at OD₃₃₂ were measured using an Ultraspec 3000 (Pharmacia Biotech). The samples were diluted 1/50 in extraction buffer and absorbance at OD₃₃₂ was read A standard curve was prepared using sinapine bisulfate. Concentrations of total phenolics absorbing at OD₃₃₂, total phenolics/fresh weight (FW) and sinapine/FW were determined as follows:
Total phenolic (μg/ml)=OD₃₃₂×slope of standard curve×dilution factor
Total phenolics/FW (μg/mg)=(total phenolics×μl of extraction buffer used)/seed weight/1×10³
Sinapine/FW (μg/mg)=(sinapine % area/100)×total phenolics(μg/mg)
Lignin Extraction and Quantification

Approximately 50 mg of ground seed was extracted with 1 ml of protein extraction buffer (200 mM Tris pH 7.5 using acetic acid, 1% SDS, 1 ul/ml β-mercaptoethanol) vortexed for 5 minutes, iced for 20 minutes and centrifuged at 20,000 g for 5 minutes. These steps were repeated until the supernatant was clear. The pellet was resuspended in 80% methanol by vortexing and incubated at 80° C. overnight. Insoluble material was collected by centrifuging at 20 000 g for 5 minutes. After removal of the supernatant, the pellet was resuspended in a 1 ml mixture of H₂O, HCl, and thioglycolic acid (75:25:1 v/v). This mixture was incubated at 80° C. for 3 hours. The insoluble material was collected by centrifuging at 20 000 g for 5 minutes. The pellet was washed with 1 ml of H₂O and centrifuging as before. After removal of the supernatant the pellet was resuspended in 1 ml 1M NaOH and gently agitated at room temperature overnight. Insoluble material was collected by centrifuging at 20 000 g for 5 minutes. The supernatant was transferred to a clean tube and 200 ul of HCL was added. The tubes were vortexed and incubated at 4° C. for 4 hours. Insoluble material was collected by centrifuging at 20 000 g for 5 minutes. The pellet was dissolved in 1 ml 1M NaOH. Samples were diluted 1:20 in 1M NaOH and the absorbance read at OD₂₈₀. Samples were prepared in triplicate. The average OD/mg of fresh weight of tissue was compared to that of wild-type.

Phytate Extraction and Quantification

Phytate was extracted and quantified following the protocol described in Brooks et al., (2001, which is incorporated herein by reference).

Strategy for Generating RNAi Constructs

The target DNA fragment is amplified by PCR using primers that generate products containing SpeI and AscI sites at the 5′ end and SwaI and BamHI sites at the 3′ end:

3′-SpeI-AscI-PCR PRODUCT-SwaI-BamHI-5′

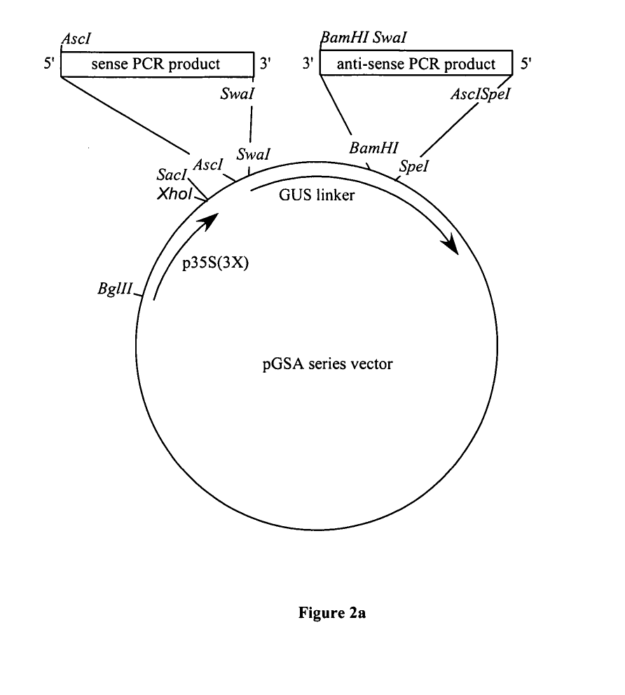

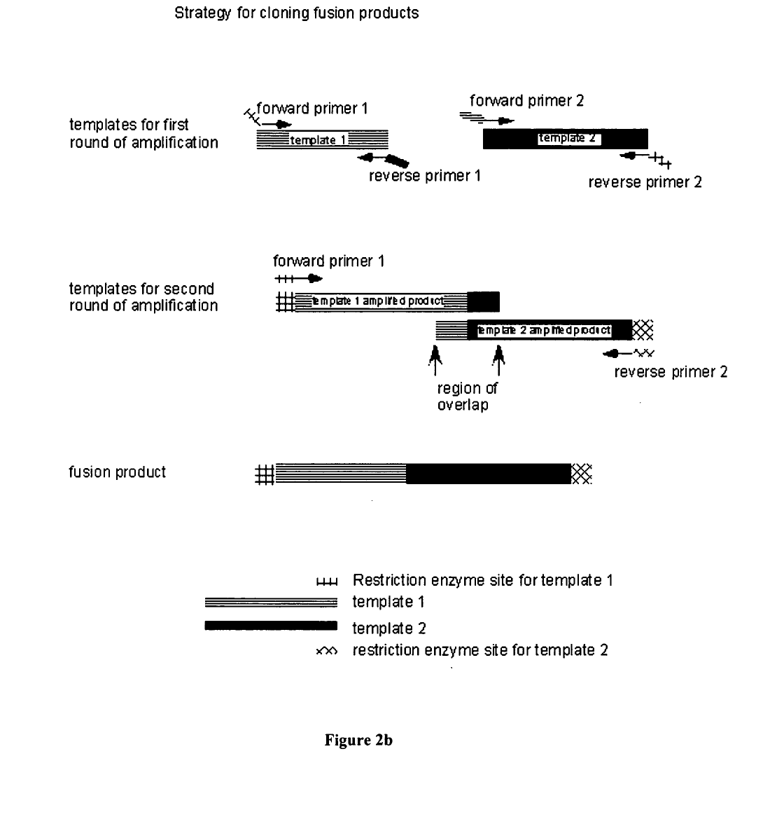

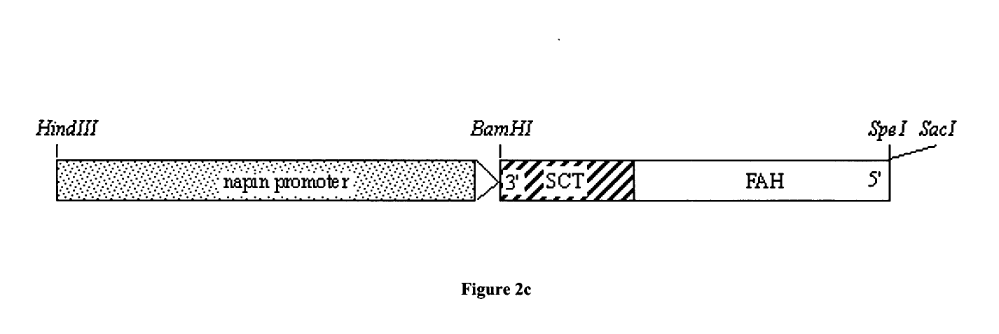

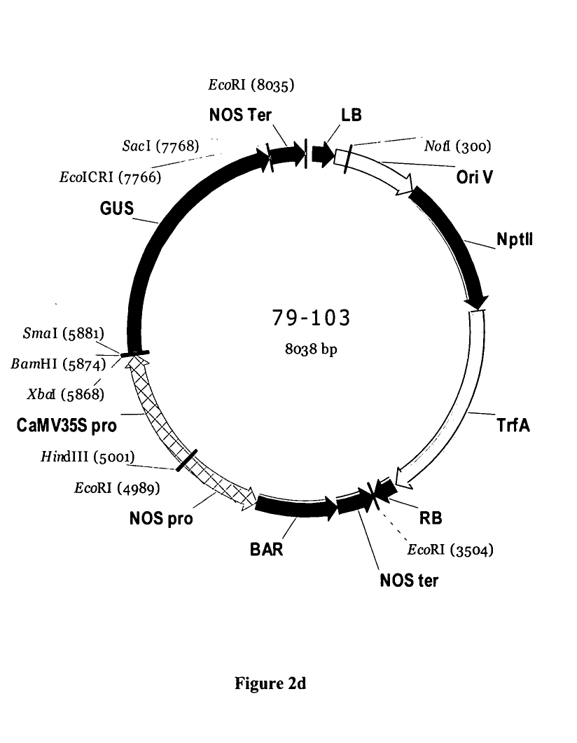

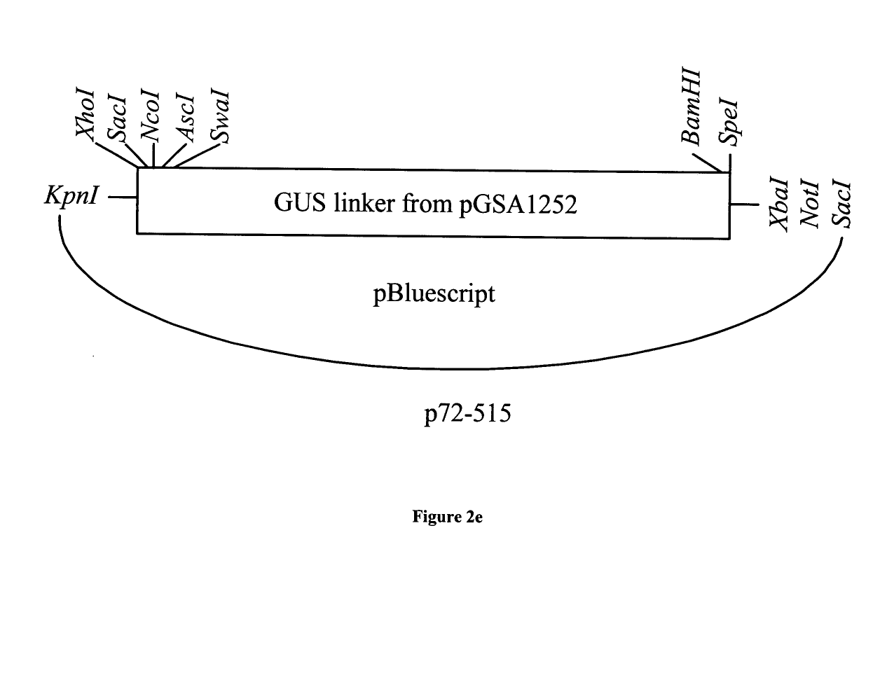

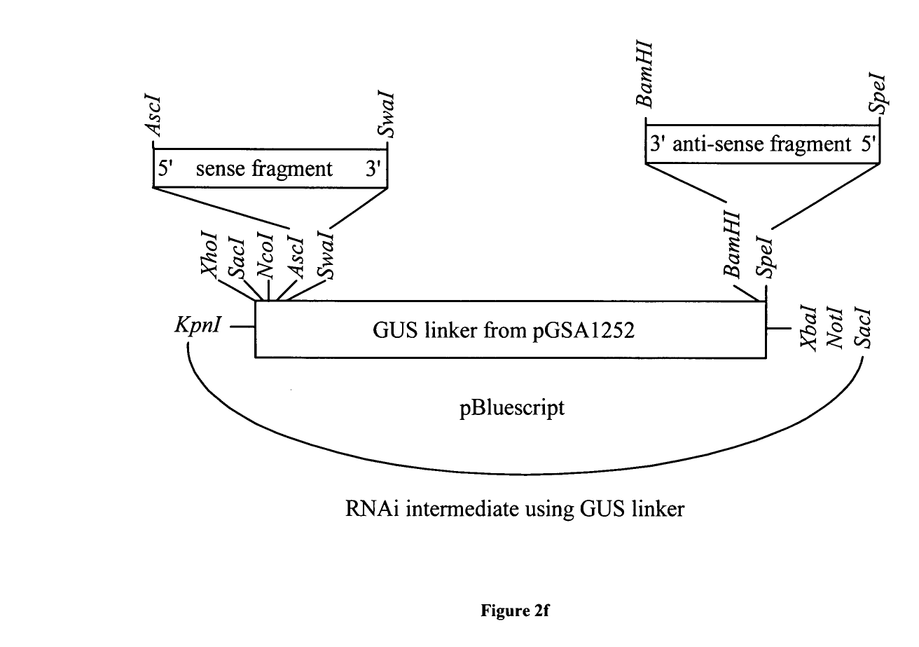

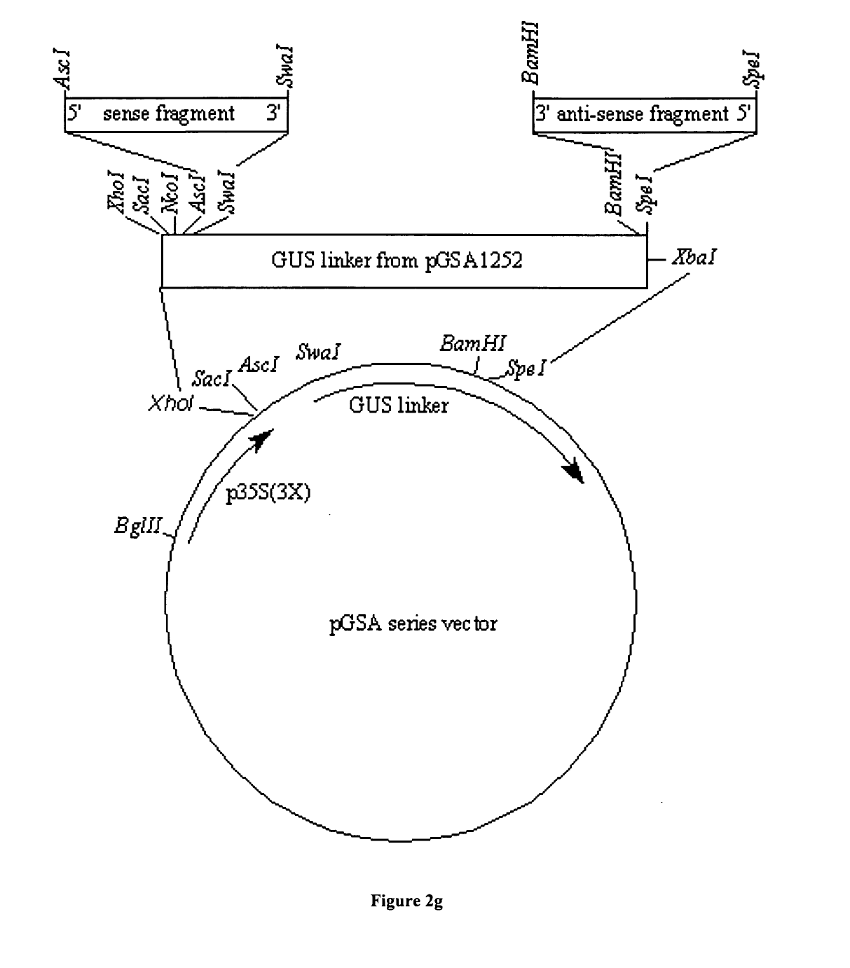

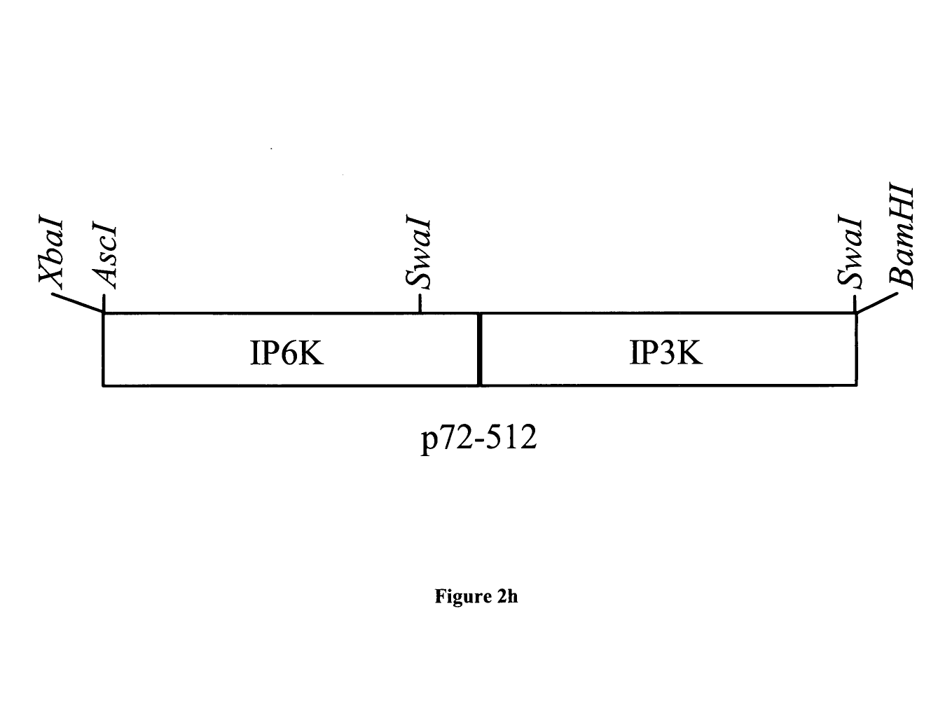

The PCR product is blunt end cloned into the EcoRV site of pBluescript producing the template vector. The template vector is digested with AscI and SwaI and ligated into the backbone pGSA1252 (SEQ ID NO: 13) or pGSA1285 (SEQ ID NO: 2) RNAi vector (obtained from Dr. Richard Jorgensen, University of Arizona) cleaved with AscI and SwaI to produce the sense orientation construct. The template vector is then digested with BamHI and SpeI and ligated into the BamHI and SpeI sites of the sense orientation construct to produce the template vector in the sense as well as the anti-sense orientation (see FIG. 2).

Strategy for Generating constructs of Gene Fusions

Template 1 and template 2 are first amplified separately using primers having the following characteristics:
TEMPLATE 1: Forward primer 1 contains two restriction enzyme sites at the 5′ end The reverse primer 1 contains 10-15 nucleotides from the 5′ end of template 2.
TEMPLATE 2: forward primer 2 contains 10-15 nucleotides from the 3′ end of template 1 and the reverse primer 2 contains two restriction enzymes sites.

The amplified template 1 and template 2 are combined and used as new templates for a new round of PCR amplification where only the forward primer 1 of template 1 and the reverse primer 2 of template 2 are used. The two separate templates are amplified to create one single in frame fusion fragment encoding template 1 and template 2 and containing restriction enzyme sites at each end. This product is then cloned into the EcoRV site of pBluescript. (see FIG. 2b)

Strategy for Generating RNAi Constructs Using the GUS Linker Cloned into pBluescript

The XhoI-SpeI GUS linker fragment from pGSA1252 was cloned into pBluescript at the XhoI-SpeI sites producing plasmid p72-515 (see FIG. 2e). This plasmid was used to create RNAi intermediates by cloning fragments into the AscI-SwaI in the sense orientation and fragments into the BamHI-SpeI sites in the anti-sense orientation (see FIG. 2f). The entire intermediate was excised via an XhoI-XbaI digest and cloned into the XhoI-SpeI sites of pGAS1252 (see FIG. 2g).

### Example 1

### Sinapine

Estimation of Gene Copy Numbers in Arabidopsis

Gene copy number is an important factor that needs to be considered when analyzing knockout mutants. Our analysis revealed that, with the exception of phenylalanine ammonia lyase (PAL), all other enzymes in the phenylpropanoid pathway are encoded by single copy genes in the Arabidopsis genome. Table 1 lists names of genes, their estimated copy numbers and T-DNA insertion mutants used below.

TABLE 1
Genes involved in the phenylpropanoid pathway, their estimated copy
numbers and Arabidopsis knockout mutants analyzed.

| | ¹copy | |
|---|---|---|
| Gene | number | Knockout lines analyzed |
| PAL (Phenylalanine ammonia | 3 | ²SALK70702, SALK92252 |
| lyase | | |
| C4H (Cinnamate 4-hydroxylase | 1 | ³SK16293 |
| C3H (Coumarate 3-hydroxylase) | 1 | SALK36132, SALK1122823 |
| COMT(Caffeic acid | 1 | SALK2373, SALK20611 |
| O-methyltransferase | | |
| FAH (Ferulic acid hydroxylase) | 1 | SALK 63792 |
| SCT (sinapoylglucose: | 1 | SALK2255, SALK18120 |
| choline sinapoyltransferase) | | |

¹Gene copy numbers were estimated using the BioVis software (see brassica.ca) set to a homology threshold of 80%.
²Lines obtained from the knockout population generated at the SALK institute (see http://signal.salk.edu/tabout.html);
³Lines obtained by screening AAFC's knockout population (see brassica.ca).

Profiling of Phenylpropanoids in Seeds of Arabidopsis Mutants

Chemical analysis carried out on seeds of wild type A. thaliana revealed that the main storage form of phenolics in the seed is sinapine, which represents over 50% of total extracted phenolics. A total of 10 Arabidopsis thaliana mutants with T-DNA inserts in genes affecting phenylpropanoid biosynthesis (FIG. 1, Table 1) were collected for this analysis. Knockouts in genes in the early steps in the pathway had no noticeable impact on sinapine accumulation. However, knockouts in genes affecting latter steps resulted in up to 90% reduction in sinapine content (FIG. 3).

Analysis of homozygous mutant lines with T-DNA inserts in pal (SALK70702 and SALK92252, c4h (SK16293) and c3h (SALK36132, SALK112823) loci had only a minimal quantitative effect on the level of sinapine, and sinapine levels similar to those observed in control plants were observed. Without wishing to be bound by theory, this could be due to alternative biosynthesis pathways, or non-specific enzymatic reactions. Knockouts in gene coding for caffeic acid O-methyltransferase (COMT), ferulic acid hydroxylase (FAH), and sinapoylglucose:choline sinapoyltransferase (SCT) resulted in a dramatic decrease in sinapine levels (FIG. 3). Reduction in the sinapine content of seeds of these three knockout mutants also resulted in the accumulation of some intermediate phenolic compounds, such as ferulic acid and sinapoylglucose (data not shown).

Homologs of several Brassica napus genes encoding enzymes involved in the synthesis of sinapine were cloned, including caffeic acid O-methyltransferase (COMT), sinapoylglucose: choline sinapoyltransferase (SCT), S-adenosylmethionine synthase (SAMS) and ferulic acid hydroxylase (FAH). These genes were used to generate RNAi and antisense constructs for expression in B. napus. Seeds of transgenic B. napus lines expressing these constructs were harvested, and subjected to analysis of sinapine levels as described above.

Constructs

CaMV35S::COMT RNAi Construct (p72-115)

The COMT fragment was amplified by PCR from the Brassica EST, ESS2134, (SEQ ID NO:1; see: brassica.ca) using the following primers:

Forward primer OMT-F5 having built-in SpeI and
AscI restriction sites:

| | |
|---|---|
| 5′-gcACTAGT [symbol] ATTGCATTATGCTAGC | (SEQ ID NO:25) |
| TCACAACCCTG-3′, | |
| and | |

reverse primer OMT-R6 having built-in BamHI and
SwaI restriction sites:

| | |
|---|---|
| 5′-gcGGATCC [symbol] aacaaagacggtgaag | (SEQ ID NO:26) |
| tagacgtacc-3′. | |

The strategy for the production of RNAi constructs outlined above was employed (see “Strategy for generating RNAi constructs”, and FIG. 2) using pGSA1285 as the backbone vector to create p72-115.
CaMV35S::SCT RNAi Construct (p72-145)

A B. napus SCT (BnSCT) clone identical to the one described in Milkowski et al., (2004) (Accession # AY383718) was isolated by screening a seed-specific library of B. napus using the A. thaliana SCT as a heterologous probe. Alignment of BnSCT to members of the serine carboxypeptidase family of proteins (Shirley et al., 2001) revealed that it had a unique exon region from 876 to 1043. Therefore, the DNA fragment encoding this region was used in making a BnSCT-specific RNAi construct (FIG. 4a).

An Arabidopsis genomic DNA fragment of ˜800 bp was amplified by PCR using the following two primers:

| | |
|---|---|
| SNG2-F1: | |
| 5′-gcggaagcctttaagactattg, | (SEQ ID NO:27) |
| and | |
| SNG2-R2: | |
| 5′ CAT GGG ATG GGA CTT ATT TCA GAT. | (SEQ ID NO:28) |

The primers were designed based on the Arabidopsis SCT sequence (At5g09640). The PCR fragment was used as a heterologous probe to screen a seed-specific cDNA library of Brassica napus. The isolated clone, p72-505 (SEQ ID NO:4), which was similar to the Arabidopsis SCT (At5g09640), was used as a template to PCR-amplify a 170 bp SCT-specific DNA fragment using the following two primers:

SCT-F7 forward primer having built-in SpeI (bold)
and AscI (underlined) restriction sites:

| | |
|---|---|
| 5′-gcactagtggcgcgccatcagtgtatctcaga | (SEQ ID NO:29) |
| gatatacatagagcag-3′ | |
| and | |

SCT-R8 reverse primer having built-in BamHI (bold)
and SwaI (underlined) restriction sites:

| | |
|---|---|
| 5′-gcggatccatttaaatagcagcttggagqagg | (SEQ ID NO:30) |
| caacgatgatg-3′. | |

The strategy for the production of RNAi constructs outlined above was employed (“Strategy for generating RNAi constructs”, and FIG. 2) using pGSA1285 as the backbone vector to generate p72-145.
Napin::SAMS RNAi Construct (p72-135)

A fragment encoding SAMS (S-adenosylmethionine synthase) was amplified by PCR from the Brassica EST, RL103, (SEQ ID NO:7; see brassica.ca) using the following primers:

Forward primer SAMS-F5 having built-in SpeI and
AscI restriction sites:

| | |
|---|---|
| 5′-gcACTAGT [symbol] agaacgggacttgcgc | (SEQ ID NO:41) |
| ttggcttagacc3′, | |
| and | |

reverse primer SAMS-R6 having built-in BamHI and
SwaI restriction sites:

| | |
|---|---|
| 5′-gcggatcc [symbol] GAACACAGACAACGGC | (SEQ ID NO:42) |
| TCAGGGACACCAA-3′. | |

The strategy for the production of RNAi constructs was employed using pGSA1285 as the backbone vector (see FIG. 2a). The CaMV35S promoter was replaced with the napin promoter (SEQ ID NO:8) via a BglII-SacI digest, generating p72-135.
Napin::FAH:SCT Anti-Sense Construct (p72-148)

An FAH EST, RL1627 (SEQ ID NO:3) isolated from a Brassica napus root cDNA library (see: brassica.ca) was used as template for PCR-amplification using the following two primers:

Forward FAH-F7 (containing a SpeI site in italics
and an AscI site in bold):

| | |
|---|---|
| 5′-gcactagtggcgcgccGCTCGTGAAGGCCCGT | (SEQ ID NO:31) |
| AATGACC-3′ | |
| and | |
| Reverse FAH-R9 | |
| 5′-ctgagatacaTGCGGTTTCGTGTAGGAGGAG- | (SEQ ID NO:32) |
| 3′, | |

(the bold portion is identical to sequence at the 5′-end of the SCT cDNA). This PCR product generated a FAH fragment containing an overlapping 3′ end having identical sequence to the 5′ end of the SCT (see FIG. 2b).

The SCT clone 72-505 was used as template for PCR-amplification using the following two primers:

| | |
|---|---|
| SCT-F11: | |
| 5′-gaaaccgcatgtatctcagagatatacataga | (SEQ ID NO:33) |
| gcag-3′, | |

(the bold portion is identical to 3′ end of FAH),
and SCT-R8 (containing a BamHI site (bold) and a
SwaI site underlined):

| | |
|---|---|
| 5′-gcggatccatttaaatagcagcttggaggagg | (SEQ ID NO:34) |
| caacgatgatg-3′. | |

This generated a SCT fragment containing an overlapping 5′end identical to the 3′ end of the FAH. The two PCR products were used together as templates to amplify a fusion product using FAH-F7 and SCT-R8 primers. The resulting product was blunt end cloned into the EcoRV site of pBluescript, creating p72-134 (SEQ ID NO:5). The vector p72-134 was digested with BamHI and SpeI and cloned into BamHI and SpeI sites of a digested pBluescript vector containing the napin promoter in the anti-sense orientation, to generate p72-148 (FIG. 2c). The HindIII-SacI fragment of p72-148 was cloned into the Hind III and SacI sites of p79-103, a plant transformation vector which was constructed in house (see FIG. 2d).
CaMV35S::C3H-C4H RNAi Construct (p72-146)

A C3H fragment was amplified by PCR from the Brassica EST, RL4992, (SEQ ID NO:9; see brassica.ca) using the following primers:

forward primer C3H-F3 (SpeI (bold) AscI
bold underlined):

| | |
|---|---|
| 5′-cgactagtGGCGCGCCAGAGATGATCAAGAAC | (SEQ ID NO:47) |
| CCAAGAGTG-3′ | |
| and | |
| reverse primer C3H-R4: | |
| 5′-AACTCTTCAGCTCCGAACGGAAGCAGCC-3′. | (SEQ ID NO:48) |

The bold portion corresponds to the 5′ end of the C4H portion (see FIG. 2b for creating fusion products).

The C4H fragment was amplified by PCR from the Brassica EST, RL7118, (SEQ ID NO:10; see brassica.ca) using the following primers:

forward primer C4H-F3 (the bold portion
corresponds to the 3′ end of the C3H portion):

| | |
|---|---|
| 5′-ccgttcggacCTGAAGAGTTTAGGCCCGAGAG- | (SEQ ID NO:49) |
| 3′, | |
| and | |
| reverse primer C4H-R4 (BamHI bold SwaI bold | |
| underlined): | |
| 5′-gcggatccATTTAAATTGGTGGAGTGGTGAAGG | (SEQ ID NO:50) |
| ATGTG-3′. | |

The products of these PCR reactions were used as templates to amplify a fusion product using C3H-F3 and C4H-R4 primers. This fusion was blunt end cloned into the EcoRV site of pBluescript to create p72-139 (SEQ ID NO:11). The strategy for the production of RNAi constructs was employed using pGSA1285 as the backbone vector generating p72-146.

Cruciferin::C3H-C4H Anti-Sense Construct (p72-152)

The cruciferin promoter (SEQ ID NO:6) was cloned into p79-103 (FIG. 2d) as a HindIII-XbaI fragment, replacing the CaMV35S promoter. The BamHI-SmaI fragment of p72-139 (see “CaMV35S::C3H-C4H RNAi construct p72-146”, above) was cloned into the BamHI-EcoICRI site of this new construct in the anti-sense orientation generating p72-152.

CaMV35S::FAH-COMT RNAi Construct (p72-142)

A FAH EST, RL1627 (SEQ ID NO:3), isolated from a Brassica napus root cDNA library, (see: brassica.ca), was used as template for PCR-amplification using the following two primers:

Forward FAH-F7 (containing a SpeI site in italics
and an AscI site in bold):

| | |
|---|---|
| 5′-gcactagtggcgcgccGCTCGTGAAGGCCCGT | (SEQ ID NO:31) |
| AATGACC-3′ | |
| and | |
| reverse FAH-R10 | |
| 5′-gcataatgcaTGCGGTTTCGTGTAGGAGGAG- | (SEQ ID NO:44) |
| 3′,, | |

in which the bold portion is identical to sequence at the 5′-end of the COMT fragment. This PCR product generated a FAH fragment containing an overlapping 3′ end having identical sequence to the 5′ end of the COMT (See FIG. 2b).

The COMT fragment was amplified by PCR from the Brassica EST, ESS2134, (SEQ ID NO:1; see brassica.ca) using the following primers:

Forward primer OMT1-F7 (where the bold portion
corresponds to the 3′ end of the FAH portion;
see “strategy for cloning fusion products”,
FIG. 2b):

| | |
|---|---|
| 5′-cgaaaccgcaTGCATTATGCTAGCTCACAACC | (SEQ ID NO:45) |
| CTG, | |
| and | |

reverse primer OMT-R6 (having built-in BamHI and
SwaI restriction sites):

| | |
|---|---|
| 5′-gcGGATCC [symbol] aacaaagacggtgaag | (SEQ ID NO:46) |
| tagacgtacc-3′. | |

The two PCR products were used as templates for producing a FAH-COMT fusion product containing SpeI-AscI sites at the 5′ end and SwaI-BamHI sites at the 3′ end. This fusion product was blunt end cloned into the EcoRV site of pBluescript. The strategy for the production of RNAi constructs outlined above was employed (see FIGS. 2a and 2b) using pGSA1285 as the backbone vector generating p72-142 (FIG. 10a).
Cruciferin::COMT Anti-Sense Construct (p72-14)

The COMT fragment was amplified by PCR from the Brassica EST, RL4637, (SEQ ID NO:12: see brassica.ca) using the following primers:

| forward primer COMT-F1 | |
|---|---|
| (containing SacI site, bold): | |
| 5′-gcgagctctcttcaagaccccttacccaatta | (SEQ ID NO:39) |
| cc-3′, | |
| and | |
| reverse primer COMT-R2 | |
| (containing a XbaI site, bold): | |
| 5′-gctctagagtgggtttgttagggagactacgg- | (SEQ ID NO:40) |
| 3′. | |

The PCR product was blunt end cloned into the EcoRV site of pBluescript, digested with SacI and XbaI and cloned into the SacI and XbaI sites of pBI121 (Clontech) in the anti-sense orientation. The CaMV35S promoter was replaced with the cruciferin promoter (SEQ ID NO:6) via HindIII/XbaI digestion and ligation generating p72-14.
Downregulation of SCT by RNAi: Reduction of Sinapine Levels

Analysis of Arabidopsis knockout mutants indicates that silencing of SCT gene (At5g09640) resulted in over 90% reduction in sinapine content (FIG. 3a; SCT-2255-55, SCT 2255-56, SCT 18120-11 or SCT 18120-13). Therefore, expression of the SCT gene was downregulated by RNAi to determine its impact on the accumulation of sinapine in B. napus seeds.

Sinapine levels in seeds of transgenic B. napus lines expressing an RNAi construct specific to a BnSCT (CaMV35S::SCT RNAi, p72-145) are shown in FIG. 4b. One of these lines (DE324) had a 51% reduction in sinapine compared to the wild type B. napus DH12075. Molecular analysis revealed the transcript levels of BnSCT were considerably lower in seeds of the offspring of DE324 (RS1859, RS1860, RS1861) than in seeds of the wild type line (FIGS. 4c and 4d).

Down Regulation of FAH-SCT by Antisense RNA: Reduction of Sinapine Levels

Previous work by Nair et al., (2000) showed that up to 40% reduction in sinapine of B. napus seeds was achieved by expressing as antisense RNA construct for FAH. As the above results with SCT revealed that downregulation of the gene encoding SCT led to a considerable reduction in sinapine, it was examined whether silencing both FAH and SCT would lead to a higher reduction in sinapine levels.

An anti-sense RNA construct for a fusion between fragments from the coding regions of the two genes was made (see FIG. 5a; napin::FAH:SCT, p72-148) and introduced to B. napus. Several lines with severe reductions in sinapine were identified (FIG. 5b). These include lines with reductions of 89.77% (DE363), 84% (DE368), 71.34% (DE373), 63.67 (DE362) and 61.84% (DE378). SCT expression in seeds of RS1863 to RS1866 (offspring of DE362), RS1868 (offspring of DE368), RS1869 to RS1872 (offspring of DE373) was reduced compared to the wild type (FIGS. 5c and 5d).

Silencing of C3H-C4H by Antisense RNA: Reduction of Sinapine Levels

Transformation of B. napus with a construct for the antisense RNA expression of a fusion between C3H and C4H gene under the control of the seed-specific promoter cruciferin (FIG. 6a, Cruciferin::C3H-C4H, p72-152) produced a transgenic line, DE599, with a 33% reduction in seed sinapine compared to the wild type line (FIG. 6b).

Silencing of C3H-C4H by RNAi: Reduction of Sinapine Levels

Transformation of B. napus with an RNAi construct for a fusion between C3H and C4H genes under the control of the constitutive promoter CaMV35S (FIG. 7a, CaMV35S::C3H-C4H RNAi, p72-146)) produced several lines with reduced sinapine including DE279, DE290 and DE293. Seeds obtained from line DE279 exhibited a reduction of sinapine of 36% (FIG. 7b) when compared to sinapine levels in seeds from the control plant (DH12075).

Silencing of SAMS Gene by RNAi: Reduction of Sinapine Levels

Transformation of B. napus with a RNAi construct for the SAMS gene under the control of the seed-specific promoter napin caused (FIG. 8a, Napin::SAMS RNAi, p72-135) produced several lines with reduced sinapine including DE297, DE298, and DE299. Seeds obtained from line DE 297 exhibited a reduction of sinapine of 65% when compared to sinapine levels in seeds from the wild type line (DH12075, FIG. 8b).

Silencing of COMT Gene by RNAi: Reduction of Sinapine Levels

Transformation of B. napus with an RNAi construct for the COMT gene under the control of the control of the constitutive promoter CaMV35S (FIG. 14a, CaMV35S::COMT RNAi, p72-115) produced a transgenic line, DE127, with a 17.4% reduction in seed sinapine compared to the wild type line (DH12075, FIG. 14b).

Silencing of COMT by Antisense RNA: Reduction of Sinapine Levels

Transformation of B. napus with a construct for the antisense RNA expression of a COMT gene under the control of the seed-specific promoter cruciferin (FIG. 15a, Cruciferin::COMT, p72-14) produced a transgenic line, AB438, with a 11.4% reduction in seed sinapine compared to the wild type line (FIG. 15b).

Silencing of FAH-COMT by RNAi: Reduction of Sinapine Levels

Transformation of B. napus with an RNAi construct for a fusion between FAH and COMT genes under the control of the constitutive promoter CaMV35S (FIG. 17a, CaMV35S::FAH-COMT RNAi, p72-142)) produced several lines with reduced sinapine including DE455 which exhibited a reduction of sinapine of 16.5%, and DE466 which exhibited a reduction of sinapine of 17.8%, (FIG. 17b) when compared to sinapine levels in seeds from the control plant (DH12075).

A summary of sinapine reduction data is presented in Table 2.

TABLE 2
Levels of sinapine reduction in transgenic B. napus seeds
expressing RNAi and antisense RNA constructs of genes in
the phenylpropanoid pathway

| | % Sinapine reduction (in |
|---|---|
| Construct | relation to wild type) |
| CaMV35S::COMT (RNAi) | 17 |
| CaMV35S::SCT (RNAi) | 51 |
| napin::SAMS (RNAi) | 65 |
| napin::FAH-SCT (AS) | 90 |
| Cruciferin::C3H-C4H (AS) | 33 |
| 35S::C3H-C4H (RNAi) | 36 |
| Cruciferin::COMT (AS) | 11.4 |
| CaMV35S::FAH-COMT (RNAi) | 17.8 |

Transgenic B. napus lines expressing RNAi and anti-sense (AS), either as single genes or as gene fusion constructs, for several genes involved in the sinapine pathway (see FIG. 1) showed sinapine reductions ranging from 11% to 90%.

### Example 2

### Lignin

Chemical analysis of seeds of Arabidopsis mutants deficient in the phenylpropanoid pathway (FIG. 1) revealed that knockouts in certain genes in the pathway resulted in moderate reductions in lignin content. B. napus lines were developed with impaired expression of several of the genes involved in lignin biosynthesis, including caffeic acid O-methyltransferase (COMT), ferulic acid hydroxylase (FAH), S-adenosylmethionine synthase (SAMS), cinnamic acid 4-hydroxylase (C4H) and coumaric acid 3-hydroxylase (C3H), 4-coumarate ligase (4CL), cinnamoyl CoA reductase (CCR). These genes were used either alone, or together as gene fusions, employing RNAi and anti-sense (AS) technologies to determine if reduced levels of lignin in plants expressing these constructs may be produced.

Constructs

Cruciferin::COMT RNAi (p72-123)

The cruciferin promoter was PCR amplified using forward primer:

cruc-F3 (containing BglII (bold underlined) and
HindIII (bold italics) restriction sites):

| | |
|---|---|
| 5′-gcagatct [symbol] TTGGCCCTTTAATTATGC | (SEQ ID NO:35) |
| TCTCTTTCTAATC-3′; | |
| and | |

reverse primer cruc-R4 (containing SacI, bold
underlined, and XbaI, bold italics):

| | |
|---|---|
| 5′ gcgagctc [symbol] ATTGTGTGTGTTTTGGTG | (SEQ ID NO:36) |
| ATAGATGGATGAAG-3′. | |

The PCR product was blunt end cloned into the EcoRV site of pBluescript digested with BglII and SacI. This cruciferin promoter fragment was used to replace the CaMV35S promoter from the CaMV35S::COMT RNAi construct (p72-115) generating p72-123 (FIG. 9a).
Napin::COMT Antisense Construct (p72-122)

The COMT fragment was amplified by PCR from the Brassica EST, ESS2134, (SEQ ID NO:1; see: brassica.ca) using the following primers:

| | |
|---|---|
| forward primer OMT-F1 5′ | |
| containing a BamHI site (bold): | |
| 5′-gcggatccATTGCATTATGCTAGCTCACAACC | (SEQ ID NO:37) |
| CTG-3′, | |
| and | |
| reverse primer OMT-R2 5′ | |
| (containing an XbaI site, bold): | |
| 5′-gctctagaaacaaagacggtgaagtagacgta | (SEQ ID NO:38) |
| cc-3′. | |

The fragment was blunt end cloned into pBluescript. The SacI-XbaI partial fragment was cloned between the SacI and XbaI sites of pBI121 (Clontech) in the anti-sense orientation. The CaMV35S promoter was replaced with the napin promoter (sequence ID#8) via a HindIII-XbaI digest generating p72-122 (FIG. 12a).
Cruciferin::COMT Anti-Sense Construct (p72-14)

The COMT fragment was amplified by PCR from the Brassica EST, RL4637, (SEQ ID NO:12: see brassica.ca) using the following primers:

| forward primer COMT-F1 | |
|---|---|
| (containing SacI site, bold): | |
| 5′-gcgagctctcttcaagaccccttacccaatta | (SEQ ID NO:39) |
| cc-3′, | |
| and | |
| reverse primer COMT-R2 | |
| (containing a XbaI site, bold): | |
| 5′-gctctagagtgggtttgttagggagactacgg- | (SEQ ID NO:40) |
| 3′. | |

The PCR product was blunt end cloned into the EcoRV site of pBluescript, digested with SacI and XbaI and cloned into the Sad and XbaI sites of pBI121 (Clontech) in the anti-sense orientation. The CaMV35S promoter was replaced with the cruciferin promoter (SEQ ID NO:6) via HindIII/XbaI digestion and ligation generating p72-14.
Napin::SAMS RNAi Construct (p72-135)

The SAMS fragment was amplified by PCR from the Brassica EST, RL103, (SEQ ID NO:7; see brassica.ca) using the following primers:

Forward primer SAMS-F5 having built-
in SpeI and AscI restriction sites:

| | |
|---|---|
| 5′-gcACTAGT [symbol] agaacgggacttgcgct | (SEQ ID NO:41) |
| tggcttagacc3′, | |
| and | |

reverse primer SAMS-R6 having built-
in BamHI and SwaI restriction sites:

| | |
|---|---|
| 5′-gcggatcc [symbol] GAACACAGACAACGGCT | (SEQ ID NO:42) |
| CAGGGACACCAA-3′. | |

The strategy for the production of RNAi constructs was employed using pGSA1285 as the backbone vector. The CaMV35S promoter was replaced with the napin promoter (SEQ ID NO:8) via a BglII-SacI digest generating p72-135 (FIG. 11a).
CaMV35S::FAH-COMT RNAi Construct (p72-142)

A FAH EST, RL1627 (SEQ ID NO:3) isolated from a Brassica napus root cDNA library, (see: brassica.ca) was used as template for PCR-amplification using the following two primers:

Forward FAH-F7 (containing a SpeI site in
italics and an AscI site in bold):

| | |
|---|---|
| 5′-gcactagtggcgcgccGCTCGTGAAGGCCCGTA | (SEQ ID NO:31) |
| ATGACC-3′ | |
| and | |
| reverse FAH-R10 | |
| 5′-gcataatgcaTGCGGTTTCGTGTAGGAGGAG- | (SEQ ID NO:44) |
| 3′, | |

in which the bold portion is identical to sequence at the 5′-end of the COMT fragment. This PCR product generated a FAH fragment containing an overlapping 3′ end having identical sequence to the 5′ end of the COMT (See FIG. 2b).

The COMT fragment was amplified by PCR from the Brassica EST, ESS2134, (SEQ ID NO:1; see brassica.ca) using the following primers:

Forward primer OMT1-F7, where the bold portion
corresponds to the 3′ end of the FAH portion
(see “strategy for cloning fusion products”,
FIG. 2b):

| | |
|---|---|
| 5′-cgaaaccgcaTGCATTATGCTAGCTCACAACCC | (SEQ ID NO:45) |
| TG, | |
| and | |

reverse primer OMT-R6 (having built-in
BamHI and SwaI restriction sites):

| | |
|---|---|
| 5′-gcGGATCC [symbol] aacaaagacggtgaagt | (SEQ ID NO:46) |
| agacgtacc-3′. | |

The two PCR products were used as templates for producing a FAH-COMT fusion product containing SpeI-AscI sites at the 5′ end and SwaI-BamHI sites at the 3′ end. This fusion product was blunt end cloned into the EcoRV site of pBluescript. The strategy for the production of RNAi constructs outlined above was employed (see FIGS. 2a and 2b) using pGSA1285 as the backbone vector generating p72-142 (FIG. 10a).
CaMV35S::C3H-C4H RNAi Construct (p72-146)

The C3H fragment was amplified by PCR from the Brassica EST, RL4992, (SEQ ID NO:9; see brassica.ca) using the following primers:

| | |
|---|---|
| forward primer C3H-F3 (SpeI (bold) | |
| AscI bold underlined): | |
| 5′-cgactagtGGCGCGCCAGAGATGATCAAGAACC | (SEQ ID NO:47) |
| CAAGAGTG-3′, | |
| and | |
| reverse primer C3H-R4: | |
| 5′-AACTCTTCAGCTCCGAACGGAAGCAGCC-3′. | (SEQ ID NO:48) |

The bold portion corresponds to the 5′ end of the C4H portion (see FIG. 2b for creating fusion products).

The C4H fragment was amplified by PCR from the Brassica EST, RL7118, (SEQ ID NO:10; see brassica.ca) using the following primers:

forward primer C4H-F3 (the bold portion cor-
responds to the 3′ end of the C3H
portion):

| | |
|---|---|
| 5′-ccgttcggacCTGAAGAGTTTAGGCCCGAGA | (SEQ ID NO:49) |
| G-3′, | |
| and | |

reverse primer C4H-R4 (BamHI bold SwaI
bold underlined):

| | |
|---|---|
| 5′-gcggatccATTTAAATTGGTGGAGTGGTGAAGG | (SEQ ID NO:50) |
| ATGTG-3′. | |

The products of these PCR reactions were used as templates to amplify a fusion product using C3H-F3 and C4H-R4 primers. This fusion was blunt end cloned into the EcoRV site of pBluescript to create p72-139 (SEQ ID NO:11). The strategy for the production of RNAi constructs was employed using pGSA1285 as the backbone vector generating p72-146 (FIG. 7a).

CaMV35S::4CL RNAi Construct (p72-170)

The 4CL fragment was amplified by PCR from the Brassica EST, RL4142, (SEQ ID NO:14; see brassica.ca) using the following primers:

| | |
|---|---|
| forward primer 4CL-F5 (SpeI bold, | |
| AscI bold underlined): | |
| 5′-gcactagtggcgcgccACCCGGCGGCTACATCA | (SEQ ID NO:51) |
| GAGACC-3′ | |
| and | |
| reverse primer 4CL-R6 (BamHI bold, | |
| SwaI bold underlined): | |
| 5′-ctggatccatttaaaTCACAACAAACGCAACGG | (SEQ ID NO:52) |
| GAACTTC-3′. | |

The PCR product was blunt-end cloned into the EcoRV site of pBluescript producing plasmid p72-165. The strategy for the production of RNAi constructs was employed using pGSA1252 as the backbone vector generating p72-170.
CaMV35S::CCR RNAi Construct (p72-174)

The CCR fragment was amplified by PCR from the Brassica EST, RL2419, (SEQ ID NO:15; see brassica.ca) using the following primers:

| | |
|---|---|
| forward primer CCR-F5 (SpeI bold, | |
| AscI bold underlined): | |
| 5′-ACACTAGTGGCGCGCCAGACGGCGAAGGAGAAA | SEQ ID NO: 53) |
| GGTGTTGA-3′ | |
| and | |
| reverse primer CCR-R6 (BamHI bold, | |
| SwaI bold underlined): | |
| 5′-caggatccatttaaatGGCTTGGCTCTTGGGTT | (SEQ ID NO:54) |
| CTTCTCGTC-3′. | |

The PCR product was blunt end cloned into the EcoRV site of pBluescript producing plasmid p72-164. The strategy for the production of RNAi constructs was employed using pGSA1252 as the backbone vector generating p72-174.
CaMV35S::4CL-CCR RNAi Construct (p72-173)

Plasmid p72-165 was PCR amplified using:

| | |
|---|---|
| forward primer 4CL-F5 (SpeI bold, | |
| AscI bold underlined): | |
| 5′-gcactagtggcgcgccACCCGGCGGCTACATCA | (SEQ ID NO:51) |
| GAGACC-3′, | |
| and | |
| reverse primer 4CL-R7 (underlined | |
| portion corresponds to the 5′ end | |
| of the CCR fragment): | |
| 5′-CTT CGC CGT CTTCACAACAAACG-3′. | (SEQ ID NO:56) |

Plasmid p72-164 was PCR amplified using:

| | |
|---|---|
| forward primer CCR-F8 (underlined | |
| portion corresponds to the 3′ end | |
| of the 4CL): | |
| 5′-CGTTTGTTGTGAAGACGGCGAAGGAGAAA | (SEQ ID NO:57) |
| G-3′, | |
| and | |
| reverse primer CCR-R6 (BamHI bold, | |
| SwaI bold underlined): | |
| 5′-caggatccatttaaatGGCTTGGCTCTTGGGTT | (SEQ ID NO:55) |
| CTTCTCGTC-3′. | |

The products of these PCR reactions were used as templates to amplify a fusion product using 4CL-F5 and CCR-R6 primers (see “Strategy for cloning fusion products, FIG. 2b). This fusion was blunt end cloned into the EcoRV site of pBluescript to create p72-168 (SEQ ID NO:16). The strategy for the production of RNAi constructs was employed using pGSA1252 as the backbone vector generating p72-173.

Reduction in Lignin Levels

Transgenic B. napus lines expressing RNAi and anti-sense (AS) constructs for several genes involved in lignin biosynthesis, including caffeic acid O-methyltransferase (COMT), ferulic acid hydroxylase (FAH), S-adenosylmethionine synthase (SAMS), cinnamic acid 4-hydroxylase (C4H) and coumaric acid 3-hydroxylase (C3H), 4-coumarate ligase (4CL), cinnamoyl CoA reductase (CCR), and gene fusions of these genes, showed lignin reductions ranging from 10% to 36% (Table 3).

TABLE 3
Levels of lignin reduction in transgenic B. napus seeds
expressing RNAi and anti-sense RNA constructs of
genes in phenylpropanoid pathway

| | % Lignin reduction (in relation to |
|---|---|
| Construct | wild type) |
| Cruciferin::COMT (RNAi) | 34 |
| CaMV35S::FAH-COMT (RNAi) | 36 |
| napin::SAMS (RNAi) | 17 |
| Cruciferin::COMT (AS) | 10 |
| Napin::COMT (AS) | 23 |
| CaMV35S::C3H-C4H (RNAi) | 29 |
| CaMV35S::4CL (RNAi) | tbd |
| CaMV35S::CCR (RNAi) | tbd |
| CaMV35S::4CL-CCR (RNAi) | tbd |

Silencing of COMT Using RNAi
Transforming Brassica napus with Cruciferin::COMT RNAi construct (FIG. 9a, p72-123) caused a reduction in lignin in many of the transgenic plants, with levels of lignin reduction of 34% (DE136 and DE138) and 33% (DE167) relative to wild type (DH12075, FIG. 9b).
Silencing of FAH and COMT Expression Using RNAi

Transforming Brassica napus with CaMV35S::FAH-COMT RNAi construct (FIG. 10a, p72-142) resulted in a reduction in seed lignin content in many of the transgenic plants, with level lignin reduction of 36% in line DE463 and 31% in line DE248 relative to the wild type DE12075 (FIG. 10b).

Silencing SAMS Gene Using RNAi

Transforming Brassica napus with napin::SAMS RNAi construct (FIG. 11a, p72-135) caused a reduction in seed lignin in several of the transgenic plants, with a reduction in the level of lignin of 17% in DE303 relative to the wild type DE12075 (FIG. 11b).

Silencing COMT Gene Expression Using Antisense RNA

Transforming Brassica napus with napin::COMT anti-sense construct (FIG. 12a, p72-122) resulted in a reduction in seed lignin in several of the transgenic plants, with a reduction in the level of lignin of 23% in DE108 and 10%-12% in lines DE42, DE103, DE104, DE105, DE111 relative to the lignin levels in the wild type DE12075 (FIG. 12b).

Silencing COMT Gene Expression Using Antisense RNA

Transforming Brassica napus with cruciferin::COMT anti-sense construct (FIG. 16a, p72-14) resulted in a reduction in seed lignin in several of the transgenic plants, with a reduction in the level of lignin of 9.4% in AB437 relative to the lignin levels in the wild type DH12075 (FIG. 16b).

Silencing of C3H and C4H Genes Using RNAi

Transforming Brassica napus with a CaMV35S::C3H-C4H RNAi construct (FIG. 13a, p72-146) resulted in several transgenic lines with reduced seed lignin content, including a reduction in lignin levels of 29% (DE276) and 21% (DE272 and DE490) relative to the lignin levels in the wild type DH12075 (FIG. 13b).

### Example 3

### Phytate

Analysis of seeds of Arabidopsis mutants with knockouts in genes affecting phytate biosynthesis (FIG. 14) revealed that knockouts in individual genes caused only moderate quantitative reduction in phytate levels, due to the high copy numbers of most genes (Table 4). Without wishing to be bound by theory, significant reduction in levels of phytate may require down-regulating entire gene families simultaneously.

TABLE 4
Genes involved in the phenylpropanoid pathway, their estimated copy
numbers. Gene copy numbers were estimated using the BioVis
software (www.brassica.ca) set to a homology threshold of 80%.

| | Estimated gene |
|---|---|
| Gene | copy number |
| 1-phosphatidylinositol-4,5-bisphosphate | 3 |
| phosphodiesterase (PIBP PDE) | |
| phosphatidylinositol phophatidylcholine transfer | 12 |
| protein (PI/PC TP) | |
| inositol 1,3,4-trisphosphate 5/6-kinase (IP3K) | 1 |
| inositol polyphosphate 5-phosphatase II (IPP) | 2 |
| phosphatidylinositol-4-phosphate 5-kinase (PIKa) | 9 |
| CDP-diacylglycerol—inositol 3- | 2 |
| phosphatidyltransferase (phosphatidylinositol | |
| synthase) (PIS) | |
| inositol polyphosphate 6-/3-/5-kinase 2b (IPK2a | 2 |
| &IPK2b) | |
| inositol polyphosphate 5′-phosphatase I (IPP) | 3 |
| Myo-inositol-1-phosphate synthase (MIP) | 3 |
| phosphatidylinositol kinase (PIPK) | 2 |
| phosphatidylinositol 3-kinase (PI3K) | 1 |
| Myo-inositol monophosphatase (MIM) | 1 |
| phosphatidylinositol 3- and 4-kinase (PIKb) | 2 |

B. napus homologs of candidate target genes, including inositol trisphosphate kinase (IP3K), inositol hexaphosphate kinase (IP6K), and phosphatidylinositol phosphate kinases (PIKs), were cloned and used alone or in combination as gene fusions to generate RNAi constructs for expression in B. napus, and level of phytate determined in transgenic plants.

CaMV35S::PIKₐ-PIK_{b} RNA Construct (p72-528)

The PIKₐ fragment was amplified by PCR from the Brassica EST, CL396R (see brassica.ca; SEQ ID NO:17, which has strong homology to phosphatidylinositol-4-phosphate-5-kinase) using the following primers:

| | |
|---|---|
| forward primer PIK-F11 (XbaI bold, | |
| AscI bold underlined): | |
| 5′-GCtctagaggcgcgccATCTCGCAATATGAAAA | (SEQ ID NO:58) |
| CTC-3′ | |
| and | |
| reverse primer PIK-R20 (bold portion | |
| corresponds to the 5′ end of the PIK | |
| from Brassica EST ESS1196F) | |
| 5′-CCAGACGATCACCCATCTTGTCTCCTGTA | (SEQ ID NO:59) |
| T-3′. | |

The PIK_{b} fragment was amplified by PCR from the Brassica EST, ESS1196F (see brassica.ca; SEQ ID NO:18, which has strong homology to phosphatidylinositol 3- and 4-kinase) using the following primers:

| | |
|---|---|
| forward primer PIK-F19 (bold portion | |
| corresponds to the 3′ end of the PIK | |
| from Brassica EST CL396R): | |
| 5′-CAAGATGGGTGATCGTCTGGTTAGTGAA-3′, | (SEQ ID NO:60) |
| and | |
| reverse primer PIK-R16 (BamHI bold, | |
| SwaI bold underlined): | |
| 5′-GCggatccatttaaatGCTTTAGCAGAGGAGA | (SEQ ID NO:61) |
| T-3′. | |

Both of the above mentioned PCR products were used as templates for the creation of a PIKₐ-PIK_{b}_{_{—}}fusion product using PIK-F11 and PIK-R16 primers (following the method outlined in FIG. 2b). This fusion was blunt end cloned into the EcoRV site of pBluescript to create p72-514 (SEQ ID NO:19). The AscI-SwaI fragment as well as the BamHI-XbaI fragment of p72-514 was cloned into the AscI-SwaI and BamHI-SpeI sites respectively of p72-515 generating the PIKₐ-PIK_{b} fusion in the sense and anti-sense orientation separated by the GUS linker (see FIG. 2f, cloning RNAi intermediates using GUS linker). This RNAi intermediate was digested with XhoI and XbaI and cloned between the XhoI and SpeI sites of pGSA1252 vector (FIG. 2g) generating p72-528.
Actin2::pGSA1252 Construct

The Arabidopsis actin2 promoter (SEQ ID NO:21) was PCR amplified using:

| | |
|---|---|
| forward primer act-F5 (HindIII bold, | |
| BamHI underlined): | |
| 5′-GCaagcttggatccATGTATGCAAGAGTC-3′ | (SEQ ID NO:62) |
| and | |
| reverse primer act-R6 (XbaI bold, | |
| XhoI underlined): | |
| 5′-GCtctagactcgagATCAGCCTCAGCCAT-3′, | (SEQ ID NO:63) |

and blunt end cloned into the EcoRV site of pBluescript producing p72-518. The BamHI-XhoI fragment from p72-518 was cloned into pGSA1252 at the BglII-XhoI sites thereby replacing the CaMV35S promoter with the actin2 promoter.
Actin2::PIKₐ RNAi Construct (p72-537)

The PIKₐ fragment was amplified by PCR from the Brassica EST, CL396R, (SEQ ID NO:17, see brassica.ca) using the following primers:

| | |
|---|---|
| forward primer PIK-F11 (XbaI bold, | |
| AscI bold underlined): | |
| 5′ GCtctagaggcgcgccATCTCGCAATATGAAAA | (SEQ ID NO:58) |
| CTC-3′ | |
| and | |
| reverse primer PIK-R12 (BamHI bold | |
| SwaI underlined): | |
| 5′-GCggatccatttaaatACCCATCTTGTCTCCTG | (SEQ ID NO:64) |
| TAT-3′, | |

and blunt end cloned into the EcoRV site of pBluescript generating p72-509 (SEQ ID NO:20). This construct was digested with either AscI-SwaI or BamHI-XbaI cloned into p72-515 AscI-SwaI or BamHI-SpeI sites in the sense and anti-sense orientation (see FIG. 2f, cloning RNAi intermediates using the GUS linker). The resulting construct was digested with XhoI-XbaI and cloned into actin2::pGSA1252 at the XhoI-SpeI sites (FIG. 2g) generating p72-537.
Actin2::IP6K RNAi Construct (p72-536)

Brassica napus genomic DNA fragment corresponding to IP6K (myo-inositol hexaphosphate kinase) was PCR amplified using:

| | |
|---|---|
| forward primer IP6K-F5 (XbaI bold, | |
| AscI underlined): | |
| 5′-CCtctagaggcgcgccTTCCAGAACACATCCA | (SEQ ID NO:65) |
| TA-3′ | |
| and | |
| reverse primer IP6K-R6 (BamHI bold, | |
| SwaI underlined): | |
| 5′-CGggatccatttaaatGATCATACACTTCGAA | (SEQ ID NO:66) |
| ACCA-3′. | |

The PCR product was blunt end cloned into the EcoRV site of pBluescript producing p72-507 (SEQ ID NO:22). This construct was digested with AscI-SwaI and cloned into the AscI-SwaI sites of p72-515 in the sense orientation producing an IP6K sense intermediate. P72-507 was then digested with BamHI-XbaI and cloned into the BamHI-SpeI sites of the IP6K sense intermediate. (see FIG. 2f, cloning RNAi intermediates using the GUS linker). The resulting construct was digested with XhoI-XbaI and cloned into actin2::pGSA1252 at the XhoI-SpeI sites (FIG. 2g) generating p72-536.
Actin2::IP6K-IP3K RNAi Construct (p72-535)

Plasmid p72-507 (SEQ ID NO:22) was PCR amplified using:

| | |
|---|---|
| forward primer IP6K-F5 (XbaI bold, | |
| AscI underlined): | |
| 5′-CCtctagaggcgcgccTTCCAGAACACATCCA | (SEQ ID NO:65) |
| TA-3′ | |
| and | |
| reverse primer IP6K-R11 (bold | |
| portion corresponds to the 5′ end | |
| of RL1344R): | |
| 5′-CCATTTAAATGATCATACACTTCG-3′. | (SEQ ID NO:67) |

The IP3K fragment was amplified by PCR from the Brassica EST, RL1344R (SEQ ID NO:23, see brassica.ca, this sequence has strong homology to inositol 1,3,4-trisphosphate 5/6-kinase) using the following primers:

| | |
|---|---|
| forward primer IP3K-F12 (bold | |
| portion corresponds to the 3′ end | |
| of p72-507): | |
| 5′-CATTTAAATGGTCGCGGAGAAGAAGCAG | (SEQ ID NO:68) |
| and | |
| reverse primer IP3K-R10 (BamHI site | |
| in bold, SwaI site in italics): | |
| 5′-GCggatccatttaaatGGGCTTAGCTATCACCG | (SEQ ID NO:69) |
| GAAACTC-3′. | |

The two PCR products were used as templates for the generation of a fusion product (see strategy for cloning fusion products, FIG. 2b) using forward primer IP6K-F5 and reverse primer IP3K-R10. The PCR fusion product was blunt end cloned into the EcoRV site of pBluescript generating p72-512 (SEQ ID NO:24; FIG. 2h). This construct was digested with BamHI, blunt ended with Klenow fragment, and then digested with AscI. The resulting fragment was cloned in the sense orientation into p72-515 digested with SwaI and blunt ended with Klenow fragment, and then digested with AscI producing an IP6K-IP3K sense intermediate.

P72-512 was also digested with BamHI-XbaI and cloned into the BamHI-SpeI sites of the IP6K-IP3K intermediate producing the antisense intermediate (see FIG. 2f, cloning RNAi intermediates using the GUS linker). The resulting construct was digested with XhoI-XbaI and cloned into actin2::pGSA1252 at the XhoI-SpeI sites (FIG. 2g) generating p72-535.

Using this strategy, phytate levels in B. napus seeds by up to 29.4% (Table 5). Additional reduction in phytate levels may be achieved by silencing multiple gene families through gene stacking.

TABLE 5
Levels of phytate reduction in transgenic B. napus seeds
expressing RNAi and antisense RNA constructs of genes
in the phytate biosynthesis pathway. PIKₐ,
phosphatidylinositol-4-phosphate-5-kinase; PIK_{b},
phosphatidylinositol 3- and 4-kinase; IP6K, myo-inositol
hexaphosphate kinase.

| | % reduction |
|---|---|
| RNAi Constructs | (in relation to wild type) |
| Actin2::IP3K-IP6K | 19 |
| Actin2::IP6K | 16.5 |
| CaMV35S::PIKₐ-PIK_{b} | 13 |
| Actin2::PIKₐ | 29.4 |

Most of the seed phytate is synthesized through the step-wise phosphorylation of myo-inositol rather than through phosphatidyl inositol. An alternate strategy to reduce phytate levels involves diverting more myo-inositol to phosphatidyl inositol biosynthesis, combined with blocking the de-esterification of the latter to myo-inositol phosphate. This is achieved by overexpressing phosphatidyl inositol synthase (PIS) and silencing phosphotidyl inositol bisphosphate phosphodiesterase (PIBP PDE).

### REFERENCES

Anderson (1985) In H. Sorensen, (ed.) Advances in the production and utilization of cruciferous crops. Martinus Nijhoff Publ., Dordrecht, The Netherlands, pp. 218.
Anderson and Sorensen (1985) In H. Sorensen, (ed.) Advances in the production and utilization of cruciferous crops. Martinus Nijhoff Publ., Dordrecht, The Netherlands, pp. 208.
Bate N J, Orr J, Ni W, Meromi A, Nadler-Hassar T, Doerner P W, Dixon R A, Lamb C J, Elkind Y. (1994) Quantitative relationship between phenylalanine ammonia-lyase levels and phenylpropanoid accumulation in transgenic tobacco identifies a rate-determining step in natural product synthesis. Proc Natl Acad Sci USA. 91: 7608-7612.
Bell J. M. (1993) Nutritional evaluation of dehulled canola meal fed to growing swine. In: 10^{th} Project Report, Research on Canola Seed, Oil and Meal. Canola Council of Canada, Winnipeg, MB.
Bilodeau P, Lafontaine J-G, and Bellemare G. (1994) Far upstream activating promoter regions are responsible for expression or the BnC1 cruciferin gene from Brassica napus. Plant Cell 14: 125-130.
Bouchereau A., Hamelin J, Lamour I, Renard M. and Larher F. (1991) Distribution of sinapine and related compounds in seeds of Brassica and allied genera, Phytochemistry, 187330: 1873-1881.
Brooks S. P. J., Oberleas D., Dawson B. A., Bleonje B. & Lampi B. J. (2001). Proposed phytic acid standard including a method for its analysis. J. A. O. C. Int., 84, 1125-1129.
Chapple C. C. S., Shirley B. W. Zook M. Hanmerschmidt R. Somerville S. S. (1994) Secondary metabolism in ArabidopsisIn Meyerowitz E M and Somerville C R (eds.) Arabidopsis, Cold Spring Harbor Laboratory, Cold Spring harbor, N.Y., pp. 989-1030.
Chapple C C, Vogt T, Ellis B E, Somerville C R. (1992) An Arabidopsis mutant defective in the general phenylpropanoid pathway. Plant Cell. 4: 1413-1424.
Cromwell G. L., Coffey R. D., Parker G. R., Monegue, H. J. and Randolph J. H. (1995) Efficacy of a Recombinant-Derived Phytase in Improving the Bioavailability of Phosphorus in Corn-Soybean Meal Diets for Pigs. J. Anim. Sci., 73: 2000-2008.
Golovan S P, Hayes M A, Phillips J P, Forsberg C W (2001a) Transgenic mice expressing bacterial phytase as a model for phosphorus pollution control. Nat. Biotechnol. 19, 429-433.
Golovan S P, Meidinger R. G., Ajakaiye A., Cottrill M., Wiederkehr M. Z., Barney D. J, Plante C., Pollard J. W., Fan M. Z., Hayes M. A., Laursen J, Hjorth J. P., Hacker R. R., Phillips J. P. and Forsberg C. W. (2001b) Pigs expressing salivary phytase produce low-phosphorus manure. Nat. Biotech., 19: 741-745.
Grand C. Parmentier P., Boudet A. Boudet A. M. (1985) Comparison of lignins and of enzymes involved in lignification in normal and brown midrib (bm3) mutant corn seedlings. Physiol. Veg., 23: 905-911.
Halpin C., M. E. Knight, G. A. Foxon, M. M. Campbell, A. M. Boudet, J. J. Boon, B. Chabbert, M-T Tollier, W. Schuch (1994) Manipulation of lignin quality by downregulation of cinnamyl alcohol dehydrogenase. Plant J., 6, 339-???.
Higgs et al. (1994), In Lessa D. and Lim C. (eds.) Nutrition and Utilization Technology in Aquaculture. American Oil Chemists' Society, Champaigne Ill., USA, pp. 130.
Hilton, J. W., Atkinson, J. L. & Slinger, S. J. (1983) Effect of increased dietary fiber on growth of rainbow trout (Salmo gairdneri). Can J. Fish.Aquat. Sci 40, 81-85.
Hobson-Frohock A., Fenwick, G. R. Heany R. K. Land D. G. Curtis R. F. (1977) Rapeseed meal and egg taint: association with sinpaine. Br. Poult. Sci. 18: 539-541.
Husken A, Baumert A, Milkowski C, Becker H C, Strack D, Möllers C. (2005) Resveratrol glucoside (Piceid) synthesis in seeds of transgenic oilseed rape (Brassica napus L.). Theor. Appl. Genet. 111: 1553-1562.
Landry L. G., Chapple C. C., and Last R L (1995) Arabidopsis mutants lacking phenolic sunscreens exhibit enhanced ultraviolet-B injury and oxidative damage. Plant Physiol. 109: 1159-1166.
Larsen et al (1983) In Proceedings of the Sixth International Rapeseed Conference, Paris, France, pp. 1577.
Liu J., Bollinger D. W., Ledoux D. R., Ellersieck M. R., and Veum T. L. (1997) Soaking Increases the Efficacy of Supplemental Microbial Phytase in a Low-Phosphorus Corn-Soybean Meal Diet for Growing Pigs. J. Anim. Sci., 75: 1292-1298.
Milkowski C., Baumert A., Schmidt D., Nehlin L. and Strack D. (2004) Molecular regulation of sinapate ester metabolism in Brassica napus: expression of genes, properties of the encoded proteins and correlation of enzyme activities with metabolite accumulation. Plant J. 38: 80-92
Moloney, M., Walker, J. and Sharma, K. (1989) High efficiency transformation of Brassica napus using Agrobacterium vectors. Plant Cell Rep. 8: 238-242.
Ni W., Paiva N. L. and Dixon R. A. (1994) Reduced lignin in transgenic plants containing an engineered caffeic acid O-methyltransferase. Transgen Res., 3: 120-126.
Nair R. B., Joy, R. W. IV, Kurylo E, Shi X, Schnaider J, Datla R. S. S., Keller W. A. and Selvaraj G. (2000) Identification of a CYP84 Family of Cytochrome P450-Dependent Mono-Oxygenase Genes in Brassica napus and Perturbation of Their Expression for Engineering Sinapine Reduction in the Seeds. Plant Physiol. 123: 1623-1634.
Raboy V., Younga K. A., Dorscha J. A. and Cooket A. (2001) Genetics and breeding of seed phosphorus and phytic acid. J. Plant Physiol. 158, 489-497
Rask L, Ellerstrom M, Ezcurra I, Stalberg K, Wycliffe P (1998) Seed-specific regulation of the napin promoter in Brassica napus. J. Plant Physiol. 152: 505-599.
Regenbrecht J. and Strack D. (1985), Distribution of 1-sinapoylglucose: choline sinapoyltransferase activity in the brassicaceae. 24: 407-410.
Relf-Eckstein J.-A., Rakow G. and Raney J. P., 2003 Yellow-seeded Brassica napus—A new generation of high quality canola for Canada. In Proceedings of the 11th International Rapeseed Congress, BP9.36 pp. 458-460).
Sussman M. R., Amasino R. M., Young J. C., Krysan P. J., and
Austin-Philips S. (2000) The Arabidopsis Knockout Facility at the University of Wisconsin-Madison. Plant Physiol. 124: 1465-1467.
Shirley A. M., McMichael C. M. and Chapple C. (2001) The sng2 mutant of Arabidopsis is defective in the gene encoding the serine carboxypeptidase-like protein sinpaoylglucose:choline sinapoyltransferase. Plant J. 28: 83-94.
Sosulski F. W. (1979) Oranoleptic and nutritional effects of phenolic: review. J. Am. Oil Chem. Soc., 56: 711-715.
Stephens L. R. and Irvine R. F. (1990) Stepwise phosphorylation of myo-inositol leading to myo-inositol hexakisphosphate in Dictyostelium Nature, 346: 580-583.
Stevenson-Paulik J., Bastidas R. J., Chiou S-T, Frye R. A. and York J. D. (2005) Generation of phytate-free seeds in Arabidopsis through disruption of inositol polyphosphate kinases. Proc. Natl. Acad. Sci. USA 102, 12612-12617.
Stringham R (1971) Genetics of four hypocotyl mutants in Brassica campestris L. J Hered 62: 248-250.
Velasco L. and C. Möllers C. (1998) Nondestructive assessment of sinapic acid esters in brassica species: II. evaluation of germplasm and identification of phenotypes with reduced levels. Crop Sci. 38: 1650-1654.
Vignols F. Rigau J. Torres M. A., Capellades M., Puigdomènech P. (1995) The brown midrib3 (bm3) mutation in maize occurs in the gene encoding caffeic acid O-methyltransferase. Plant Cell, 7: 407-416.

All citations are hereby incorporated by reference.

The present invention has been described with regard to one or more embodiments. However, it will be apparent to persons skilled in the art that a number of variations and modifications can be made without departing from the scope of the invention as defined in the claims.

What is claimed is:

1. A method for reducing the level of one or more than one protein in a plant or a tissue within the plant comprising,
i) introducing a nucleic acid sequence into the plant, the nucleic acid sequence comprising a regulatory region operatively associated with a silencing nucleotide sequence, wherein expression of the silencing nucleotide sequence reduces or eliminates the expression of two or more than two enzymes involved in the synthesis of the one or more than one protein, and
ii) expressing the silencing nucleotide sequence within the plant or a tissue within the plant, to reduce the level of the one or more than one protein in the plant or within a tissue of the plant, the reduced level of the one or more than one protein is determine by comparing the level of the one or more than one protein in the plant, or a tissue of the plant, with a level of the one ore more than one protein in a second plant, or the tissue from the second plant, that does not express the silencing nucleic acid sequence.

2. The method of claim 1 wherein the regulatory region is selected from the group consisting of a constitutive regulatory region, an inducible regulatory region, a developmentally regulated regulatory region, and a tissue specific regulatory region.

3. The method of claim 2, wherein the regulatory region is a tissues specific regulatory region.

4. The method of claim 1 wherein the level of the one or more than one protein is reduced by about 25 to about 100%, where compared to the level of the same one or more than one protein obtained from second plant.

5. The method of claim 1, wherein the silencing nucleotide sequence is selected from the group consisting of an antisense RNA encoding nucleotide sequence, a ribozyme encoding sequence, and an RNAi encoding nucleotide sequence.

6. The method of claim 5, wherein the silencing nucleotide sequence is a gene fusion.

7. The method of claim 6, wherein the gene fusion comprises nucleic acid sequences encoding from two to ten gene sequences.

8. The method of claim 1 wherein the protein is involved in the synthesis of an anti-nutrient factor in seed tissue.

9. The method of claim 8, wherein the anti-nutrient factor is selected from the group consisting of sinapine, phytate, fiber, and lignin.

10. The method of claim 8, wherein the two or more than two enzymes are involved in a pathway of phenylpropanoid biosynthesis leading to sinapine synthesis, a pathway of phenylpropanoid biosynthesis leading to lignin synthesis, a pathway of phytate biosynthesis, or a combination thereof.

11. The method of claim 9 wherein the anti-nutrient factor is sinapine, and the two or more than two enzymes are involved in a pathway of phenylpropanoid biosynthesis.

12. The method of claim 11, wherein the two or more than two enzymes are selected from the group consisting of phenylalanine ammonia lyase (PAL), cinnamate 4 hydroxylase (C4H), coumarate 3 hydroxylase (C3H), O-methyl transferase (OMT), ferulic acid hydroxylase (FAH), sinapate: UDP-glucose sinapoyltransferase (SGT), sinapolyglucose:choline sinapoyltransferase (SCT), S-adenosylmethionine synthase (SAMS),

13. The method of claim 12, wherein the two or more than two enzymes are FAH and SCT.

14. The method of claim 12, wherein the silencing nucleotide sequence is selected from the group consisting of an antisense RNA nucleotide sequence encoding sequence, a ribozyme encoding sequence, and an RNAi encoding nucleotide sequence.

15. The method of claim 9 wherein the anti-nutrient factor is lignin, and the two or more than two enzymes are involved in a pathway of phenylpropanoid biosynthesis.

16. The method of claim 9 wherein the anti-nutrient factor is lignin, and the nucleotide sequence encodes two or more proteins selected from the group consisting of cinnamic acid 4 hydroxylase (C4H), coumaric acid 3 hydroxylase (C3H), caffeic acid O-methyl transferase (COMT), ferulic acid hydroxylase (FAH), S-adenosylmethionine synthase (SAMS), 4-coumarate:CoA lyase (4CL), cinnamoyl-CoA reductase (CCR), phenylalanine ammonia lyase (PAL), cinnamyl alcohol dehydrogenase (CAD), and peroxidase (POD).

17. The method of claim 16, wherein the two or more than two enzymes are selected from the group consisting of FAH-COMT, C3H-C4H, and 4CL-CCR.

18. The method of claim 17, wherein the silencing nucleotide sequence is selected from the group consisting of an antisense RNA nucleotide sequence encoding sequence, a ribozyme encoding sequence, and an RNAi encoding nucleotide sequence.

19. The method of claim 9 wherein the anti-nutrient factor is phytate, and the two or more than two enzymes are involved in a pathway of phytate biosynthesis.

20. The method of claim 9 wherein the anti-nutrient factor is phytate, and the nucleotide sequence encodes two or more proteins selected from the group consisting of inositol 1,3,4-trisphosphate 5/6-kinase (IP3K), myo-inositol hexaphosphate kinase (IP6K), 1-phosphatidylinositol-4,5-bisphosphate phosphodiesterase (PIBP PDE), phosphatidylinositol phophatidylcholine transfer protein (PI/PC TP), inositol polyphosphate 5-phosphatase II (IPP), phosphatidylinositol-4-phosphate 5-kinase (PIKa), CDP-diacylglycerol-inositol 3-phosphatidyltransferase (phosphatidylinositol synthase, PIS), inositol polyphosphate 6-/3-/5-kinase 2b (IPK2a & IPK2b), inositol polyphosphate 5′-phosphatase I (IPP), myo-inositol-1-phosphate synthase (MIP), phosphatidylinositol kinase (PIPK), phosphatidylinositol 3-kinase (PI3K), myo-inositol monophosphatase (MIM), and phosphatidylinositol 3- and 4-kinase (PIKb).

21. The method of claim 21, wherein the two or more than two enzymes are selected from the group consisting of IP3K-IP6K and PIKa-PIKb.

22. The method of claim 21, wherein the silencing nucleotide sequence is selected from the group consisting of an antisense RNA nucleotide sequence encoding sequence, a ribozyme encoding sequence, and an RNAi encoding nucleotide sequence.

23. A construct comprising a silencing nucleotide sequence, the silencing nucleotide sequence encoding two or more than two sequences that reduce or inhibit the expression of two or more that two enzymes involved in the synthesis of the one or more than one protein leading to sinapine biosynthesis, lignin biosynthesis, phytate biosynthesis, or a combination thereof.

24. A plant comprising a nucleic acid sequence comprising a regulatory region operatively associated with a silencing nucleotide sequence, the silencing nucleotide sequence reducing or eliminating the expression of two or more than two enzymes involved in the synthesis of the one or more than one protein leading to sinapine biosynthesis, lignin biosynthesis, phytate biosynthesis, or a combination thereof.

25. A method for reducing the level of one or more than one protein within a plant or a tissue within the plant comprising, expressing a nucleotide sequence within the plant or a tissue within the plant, the nucleic acid sequence comprising a regulatory region operatively associated with a silencing nucleotide sequence, where expression of the silencing nucleotide sequence reduces or eliminates the expression of two or more than two enzymes involved in the synthesis of the one or more than one protein leading to sinapine biosynthesis, lignin biosynthesis, phytate biosynthesis, or a combination thereof, the reduced level of the one or more than one protein determined by comparing the level of the protein in the plant, or a tissue of the plant, with a level of the protein in a second plant, or the tissue from the second plant, that does not express the nucleic acid sequence.

26. A seed comprising a silencing nucleic acid sequence and having a reduced level of one or more than one anti-nutrient compound, when compared to the level of the one or more anti-nutrient compound in a wild type seed.

27. The seed of claim 26 wherein the anti-nutrient compound is selected from the group of an intermediate of phenylpropanoid biosynthesis, a product of the phenylpropanoid pathway, sinapine, lignin, an intermediate of phytate biosynthesis, phytate.

28. A method for reducing the level of one or more than one protein in a plant or a tissue within the plant comprising,
i) providing a plant comprising a nucleic acid sequence, the nucleic acid sequence comprising a regulatory region operatively associated with a silencing nucleotide sequence, wherein expression of the silencing nucleotide sequence reduces or eliminates the expression of two or more than two enzymes involved in the synthesis of the one or more than one protein, and
ii) expressing the silencing nucleotide sequence within the plant or a tissue within the plant, to reduce the level of the one or more than one protein in the plant or within a tissue of the plant, the reduced level of the one or more than one protein is determine by comparing the level of the one or more than one protein in the plant, or a tissue of the plant, with a level of the one ore more than one protein in a second plant, or the tissue from the second plant, that does not express the silencing nucleic acid sequence.

29. The method of claim 1, wherein in the step of introducing, the nucleotide sequence is introduced into the plant by transformation.

30. The method of claim 1, wherein in the step of introducing, the nucleotide sequence is introduced into the plant by crossing the plant with a second plant, the second plant comprising the nucleotide sequence.


SEQUENCE LISTING (69 sequences)

SEQ ID NO: 1 - DNA, 1133 bp, Artificial sequence
  ESS2134 COMT
tgcagctagccagcgcctccgtccttcccatggttttaaagtccgcgctagaccttgatcttctcgagatcatggccaagaactcttttccgatgtctccctctgagattgcttctaaacttcagaccaaaaaccccgaagctccggtcatgctcgaccgaatcctccgtcttctcacgtcttactccatcctcacctgctccaaccgtactattcccggcggagacggcatcgagaggatttacgggcttggtccggtttgcaagtacttgaccaagaacgaagatggtgtctctatagctgctctttgtcttatgaaccaagacaaggttctcatggaaagctggtaccatttgaaagatgcaattcttgatggtgggattccatttaacaaggcttatggaatgagcgcttttgagtaccacgggaaggatctaaggttcaacacggtattcaacaatggaatgtctaaccattcaaccatcacaatgaagaagattctcgagacctacaagggttttgagggtttgacttctttggttgacgttggtggtggcattggtgctactctcaaaatgattgtctctaagtaccctgaccttaaaggcatcaactttgatctcccacatgtcatcgaagaagctacttctcatcccggtattgatcatgttggaggagatatgtttgtaagcgtccctaaaggtgatgctattttcatgaagtggatatgccacgactggagcgatgaacactgcgtgaaattcttgaagaactgctacgaggcgcttccagaggatggaaaagtgatactagcagagtgtatacttccagagacaccagactcaagcctctcgaccaaacaagtagtccatgttgattgcattatgctagctcacaaccctggaggcaaagagaggaccgagaaagagttcgaagcattggctaaaggatcaggcttcaaagggatcaatgttgcctgcaatgcttttggtgtttacgttattgagctgctcaaaaagatgtaaaacacacacacaatccatgtaataatgatattatatgtaaacattgctctcatggtacgtctacttcaccgtctttgttttaaaactactatgtttaataatggttat

SEQ ID NO: 2 - DNA, 9948 bp, Artificial sequence
  pGSA1285
  misc_feature (8600)..(8600): n is a, c, g, or t/u, unknown, or other
gaattctgagatttttcaaatcagtgcgcaagacgtgacgtaagtatccgagtcagtttttatttttctactaatttggtcgtttatttcggcgtgtaggacatggcaaccgggcctgaatttcgcgggtattctgtttctattccaactttttcttgatccgcagccattaacgacttttgaatagatacgctgacacgccaagcctcgctagtcaaaagtgtaccaaacaacgctttacagcaagaacggaatgcgcgtgacgctcgcggtgacgccatttcgccttttcagaaatggataaatagccttgcttcctattatatcttcccaaattaccaatacattacactagcatctgaatttcataaccaatctcgatacaccaaatcgaatcgatggttgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgactcatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgatgcagaatcttggactcccatgttggcaaaggcaaccaaacaaacaatgaatgatccgctcctgcatatggggcggtttgagtatttcaactgccatttgggctgaattgaagacatgctcctgtcagaaattccgtgatcttactcaatattcagtaatctcggccaatatcctaaatgtgcgtggctttatctgtctttgtattgtttcatcaattcatgtaacgtttgcttttcttatgaattttcaaataaattatcagatctctctgccgacagtggtcccaaagatggacccccacccacgaggagcatcgtggaaaaagaagacgttccaaccacgtcttcaaagcaagtggattgatgtgacatctccactgacgtaagggatgacgcacaatcccactatccttcgcaggatctctctgccgacagtggtcccaaagatggacccccacccacgaggagcatcgtggaaaaagaagacgttccaaccacgtcttcaaagcaagtggattgatgtgacatctccactgacgtaagggatgacgcacaatcccactatccttcgcaggatctctctgccgacagtggtcccaaagatggacccccacccacgaggagcatcgtggaaaaagaagacgttccaaccacgtcttcaaagcaagtggattgatgtgacatctccactgacgtaagggatgacgcacaatcccactatccttcgcaagacccttcctctatataaggaagttcatttcatttggagaggacacgctcgagtataagagctcatttttacaacaattaccaacaacaacaaacaacaaacaacattacaattacatttacaattaccatggggcgcgcccaatcgatgatttaaatccccagatgaacatggcatcgtggtgattgatgaaactgctgctgtcggctttaacctctctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaagtgcacgggaatatttcgccactggcggaagcaacgcgtaaactcgacccgacgcgtccgatcacctgcgtcaatgtaatgttctgcgacgctcacaccgataccatcagcgatctctttgatggggatcctcagcttaattaactagtccctagagtcctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaagtgccccaggggctagtatctacgacacaccgagcggcgaactaataacgctcactgaagggaactccggttccccgccggcgcgcatgggtgagattccttgaagttgagtattggccgtccgctctaccgaaagttacgggcaccattcaacccggtccagcacggcggccgggtaaccgacttgctgccccgagaattatgcagcatttttttggtgtatgtgggccccaaatgaagtgcaggtcaaaccttgacagtgacgacaaatcgttgggcgggtccagggcgaattttgcgacaacatgtcgaggctcagcaggacctgcaggcatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcagcttgagcttggatcagattgtcgtttcccgccttcagtttaaactatcagtgtttgacaggatatattggcgggtaaacctaagagaaaagagcgtttattagaataacggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcatgatatatctcccaatttgtgtagggcttattatgcacgcttaaaaataataaaagcagacttgacctgatagtttggctgtgagcaattatgtgcttagtgcatctaatcgcttgagtnaacgccggcgaagcggcgtcggcttgaacgaatttctagctagaggatcgcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccattgtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaactccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccatgatgtttaactttgttttagggcgactgccctgctgcgtaacatcgttgctgctccataacatcaaacatcgacccacggcgtaacgcgcttgctgcttggatgcccgaggcatagactgtaccccaaaaaaacatgtcataacaagaagccatgaaaaccgccactgcgccgttaccaccgctgcgttcggtcaaggttctggaccagttgcgtgacggcagttacgctacttgcattacagcttacgaaccgaacgaggcttatgtccactgggttcgtgcccgaattgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgcc

SEQ ID NO: 3 - DNA, 1728 bp, Artificial sequence
  RL1627 FAH
mggmctscgtrkscacccggtccacgtgggtggcccatcataggcaatatgtcaatgatggaccaactcacccaccgtggtttagccaacttagctaaaaagtacggtggcttgtgccatctccgcatgggatttctccacatgtacgccgtttcatcaccagacgtggctaaacaagtccttcaagtccaagacagcgtcttctcgaaccgaccagctactatagctataagctatttgacttatgaccgagccgacatggcgtttgctcactacggaccgttttggagacagatgaggaaagtttgcgtcatgaaggtgtttagccgtaaacgagccgagtcatgggcttctgttcgtgatgaagtggacaaaatgatccggtcggtttctagtaacgttggtaagtctatcaacgttggtgagcaaatttttgcactgacccgaaacataacttaccgggcagcgttcgggtcagcatgtgaaaagggacaagacgagttcataagaattttacaagagttctctaagctttttggagccttcaacgtagcggatttcataccgtatttcgggtggatcgatcctcaaggaataaacaagcggctcgtgaaggcccgtaatgacctagacggatttattgacgatatcatcgatgaacacatgaagaagaaagagaatcaaaacagtgttgatgctggagatgttgttgataccgatatggttgatgatcttcttgctttttacagtgaagaggcgaaattagtgagcgagacagcggatcttcagaactctatcaaacttacccgtgacaatatcaaagcaatcatcatggacgttatgtttggaggaacggaaacggtagcgtcagcgatagagtgggcattgactgaattactacggagccccgaggatctaaaccgagtccaacaagaactcgctgaagtcgtcggacttgaccgacgtgtggaagaatcagacatcgagaagttgacttttctgaaatgcacactcaaagaaaccctaaggctacacccaccgatcccactcctcctacacgaaaccgcagaggacactgagatcgacggttacttcgttcccaagaaatctcgcgttatgatcaacgcgtttgcgattgggcgcgacaagaactcttgggttgaccccgaaacgtttagaccgtctaggtttttggaaccgggcgtaccagatttcaaagggagtaacttcgagtttataccattcgggtcgggtcgtaggtcgtgcccgggtatgcaactcgggttatacgcgcttgaactagccgtggcccatatattacattgcttcacgtggaaattacctgatggcatgaaaccaagcgagcttgatatgagcgacgtgtttggtctgacggctcctaaagccacgcgtctctatgctgtcccgagcacgcgccttatttgttctgtttaagttatggttcgaagcacgtggcgggtgaaaggaaaggtggttggtatggttcttgaaagtggtgtgagaagtcaaaagaagccctgaagatttgtggatgttatatataatatatgtttatgtatttgtgtgttcacacgtgtgttctggatgaaacataaagtggctctttgtttcgttttccaatttcttttgtgggaattcttttccttgcatgaaatgtaaacgctgaaaaataagatttttttttacaaataaaaaaaaaaaaaaaaa

SEQ ID NO: 4 - DNA, 2121 bp, Artificial sequence
  p72-505 SCT
  misc_feature (16)..(16): n is a, c, g, or t/u, unknown, or other
  misc_feature (32)..(32): n is a, c, g, or t/u, unknown, or other
ccacgatagatgcccnatgacgtcgcatgctngcgcccaataagcttggatcctctagagcggccgctcgcgatctagaagcggagaaaatgagaaatctttactttctagtcttatttccgttgagcatcttgattttggttgatgcttctttgcatgtgaagtatcttcctggtcttgaaggtcctcttccttttgagctagagactgggtatgtgagtgttggtgaatctggagatgttgagctcttttactactttgtgaaatcagagagcaatccagataaagatcctctcatgatttggctaactggtgggcctggatgcagctcaatttgtggtttactctttgcaaatggtcctttggcttttaaaggggatgagtataatgggacactgcctcctttagagctaacatctttttcttggacaaaggtggctaacattttatatttggaatctcctgctggttctggatattcttatgccaaaactcggcgtgctgctgagacgagcgacaccaaacaaattcaccaaatcgaccagttccttaggagttggtttgtggaccaccctgagtttatatccaattcattttacgttggtggagattcatattccgggaagattgttccaggagttgtgcaacagatttcacttggaaatgaaaaaggtctcacaccactcataaatattaagggatatgttcttggaaaccctgcagtacgtacaaacttagaaccaaatcatagagtttcatttgcgcatcggatgggacttatttcagatgagctccatgagtcacttgaaagaaactgtggaggcaaattctttaacgtagatccaagtaatgcaaaatgttcaaatgggcttctagcttatcatcagtgtatctcagagatatacatagagcagattttgttaccaaactgcaaagtagattatgtcttagcagacatatcacaaaccttaccaaatatcagaaccagtcgaagaagagaactcaaggagttttcaagaaatgattcatcatcgttgcctcctccaagctgctttacttataggtattttctgtctgccttttgggcaaatgatgaaaatgtacgcagagctttaggcgtgaagaagggcttcggaaaatggagtcgatgcaacactcaaaacataccatatacatatgatattcacaatgccattccatatcacgttaataatagccgtaaaggcttccgcgctctcatctacagtggtgatcatgatatgatgatacctttctcttcaactgaagcatggatcaaatctctcaactattccattgttgatgactggagaccttggatgatgaatagcaatcaagttgctggatatacaaggacctatgcaaataagatgacatttgcaaccatcaagggaggaggacacaccgctgagtataatccagaccaatgctcacttatgttcaaaagatggattgatggtgaatctctctgaagttcctcactcacttctctctctctctctctgtcaaaagtttcatgagttgcttttgttttcgtttatggttattaagtcaaaatttaaaaataataataaccttttattaaaaaaaaaaaaaaagggcggccgcggacgcgtgggtcgacccgggaattccggaccggtacctgcaggcgtaccagctttccctatagtgagtcgtattagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgaaagagttgcagcaagcggtccacgctggtttgccccagcaggcgaaaatcctgtttgatggtggttaacggcgggatataacatgagctgtcttcggtatcgtcgtatcccact

SEQ ID NO: 5 - DNA, 679 bp, Artificial sequence
  p72-134 FAH-SCT
ggaattcgatgcactagtggcgcgccgctcgtgaaggcccgtaatgacctagacggatttattgacgatatcatcgatgaacacatgaagaagaaagagaatcaaaacagtgttgatgctggagatgttgttgataccgatatggttgatgatcttcttgctttttacagtgaagaggcgaaattagtgagcgagacagcggatcttcagaactctatcaaacttacccgtgacaatatcaaagcaatcatcatggacgttatgtttggaggaacggaaacggtagcgtcagcgatagagtgggcattgactgaattactacggagccccgaggatctaaaccgagtccaacaagaactcgctgaagtcgtcggacttgaccgacgtgtggaagaatcagacatcgagaagttgacttttctgaaatgcacactcaaagaaaccctaaggctacacccaccgatcccactcctcctacacgaaaccgcatgtatctcagagatatacatagagcagattttgttaccaaactgcaaagtagattatgtcttagcagacatatcacaaaccttaccaaatatcagaaccagtcgaagaagagaactcaaggagttttcaagaaatgattcatcatcgttgcctcctccaagctgctwtttaaatggatccgcatcaagct

SEQ ID NO: 6 - DNA, 755 bp, Artificial sequence
  cruciferin promoter
agatctaagcttttggccctttaattatgctctctttctaatcgaccttctgggggggggggggggggggtaaagtggtcaattatattctttatggatgagcttgattgagaatgggtttagggttatgacaagggcatgtacaaatgtcactgcctcttaacatgcaaccgaacagttggcgactcaagtcgcagaagatcaacggaccaaaccctcctagtgtcgccgcgtctctcaagtctcaagtgtcacctttttgtctcttttccttttgaattggtaactctttcaataatatatagttttggctgtatctcctagactgttcgatcacaaccccagatattttaatactggatactagtgatgtaatttgatacgttaattgtcaaaaagtagattctcctatcccctttattatatggttaaatgaatttaagacgattaaaaatgaaattaatcaactagagagttcctctgtactggttgataacatgaacctaaaaatgagagatacatttgaattttgtcgtggtttggagtatgcggagatagtgctacgcgcatgaacatcatgagacacttgcttcagctcacagagtgacgtgtaaagaccatagacccacgacttcatgcaaaccagttcctacgtggcaaaaccttcatgctcactccacatatataaactcctaccaagtctccatgtttcttcatccatctatcaccaaaacacacacaattctagagagctc

SEQ ID NO: 7 - DNA, 1455 bp, Artificial sequence
  RL103 SAMS
catttctctagcagtatccattactatcaccttcagattgaaagatggaatcttttttgtttacctccgagtccgtcaacgagggacaccccgacaagctctgcgaccagatctccgacgcaatcctcgacgcttgtctcgagcaagaccctgagagcaaagtcgcttgcgagacatgtaccaagaccaacatggtcatggtcttcggcgaaatcaccaccaaggccaacgtcgactacgagaagatcgtccgcaagacttgccgtgagatcggattcatctccgacgacgttggactagacgctgacaactgcaaggtcctcgtcaacatcgagcagcagagccccgacatcgctcaaggtgttcacggtcatctcaccaagaagcctgaagacatcggagctggtgaccaaggtcacatgttcggttacgccaccgacgagacgcccgagctcatgccgcttagccacgttctcgcgaccaagcttggagctaaactcactgaggttcgtaagaacgggacttgcgcttggcttagacctgacggtaagacgcaagtcaccgttgagtacttaaacgagaacggagctatggtccctgtccgcgtccacactgttctgatctcgacccagcacgatgagaccgtgaccaatgacgagatcgcagctgatctcaaggagcatgtgatcaagccagtgatcccagagaagtaccttgacgagaagacaatcttccatctcaacccttctggtcggtttgtcatcggaggtcctcacggtgacgctggactcaccgggcgtaagatcatcatcgacacttacggtggttggggcgcgcacggaggtggcgctttctccgggaaggacccgaccaaggtcgacaggagcggtgcctacatcgtgaggcaagcggctaagagtattgttgccagtgggctagcgaggcgttgcattgtgcaggtctcgtacgccattggtgtccctgagccgttgtctgtgttcgtggacagctacggaacagggaagataccagacaaggagattcttgagattgtgaaggagagttttgacttcagaccggggatgatttcgatcaacttggatttgaagagaggtggtaatggtaggttcttgaagactgctgcttatggtcattttggaagggacgatgctgacttcacctgggaggttgtgaagccactcaagtctaacaaggtccaagcttgagaagaaaaaaaaactgaactagtctctcaggtcttgcctctgtttcaactaaagagatgtgtcttattctttttccttttcagtataagcagttgagaatttatattattattatttattatcatatgttattgttgtggtaagaaagttaaaagtcatattttgtatctcttgtttcttttcagaagatatatgaaagattatattcaataaaaaaaaaaaaaaaaaa

SEQ ID NO: 8 - DNA, 1167 bp, Artificial sequence
  napin promoter
agatctaagcaagctttcttcatcggtgattgattcctttaaagacttatgtttcttatcttgcttctgaggcaagtattcagttaccagttaccacttatattctggactttctgactgcatcctcatttttccaacattttaaatttcactattggctgaatgcttcttctttgaggaagaaacaattcagatggcagaaatgtatcaaccaatgcatatatacaaatgtacctcttgttctcaaaacatctatcggatggttccatttgctttgtcatccaattagtgactactttatattattcactcctctttattactattttcatgcgaggttgccatgtacattatatttgtaaggattgacgctattgagcgtttttcttcaattttctttattttagacatgggtatgaaatgtgtgttagagttgggttgaatgagatatacgttcaagtgaatggcataccgttctcgagtaaggatgacctacccattcttgagacaaatgttacattttagtatcagagtaaaatgtgtacctataactcaaattcgattgacatgtatccattcaacataaaattaaaccagcctgcacctgcatccacatttcaagtattttcaaaccgttcggctcctatccaccgggtgtaacaagacggattccgaatttggaagattttgactcaaattcccaatttatattgaccgtgactaaatcaactttaacttctataattctgattaagctcccaatttatattcccaacggcactacctccaaaatttatagactctcatccccttttaaaccaacttagtaaacgttttttttttaattttatgaagttaagtttttaccttgtttttaaaaagaatcgttcataagatgccacgccagaacattagctacacgttacacatagcatgcagccgcggagaattgtttttcttcgccacttgtcactcccttcaaacacctaagagcttctctctcacagcacacacatacaatcacatgcgtgcatgcattattacacgtgatcgccatgcaaatctcctttatagcctataaattaactcatcggcttcactctttactcaaaccaaaactcatcaatacaaacaagattaaaaacatacacggactctagagagctc

SEQ ID NO: 9 - DNA, 903 bp, Artificial sequence
  RL4992 C3h
gccgatgagaaggcttttgctaagcacggagctcgccgtgacatcctcacgcgagctatcatggaggaacacactttggcccgtcaaaagtctagtggagctaagcagcatttcgttgatgcgttgcttacgttaaaggatcagtatgatcttagtgaagacaccatcattggtcttctatgggacatgatcacagcgggtatggacacaacagctataacagcagaatgggccatggcagagatgatcaagaacccaagagtgcaacaaaaggtacaagaagagttcgacagagttgttggactagaccgggtcgtaaccgagccagacttctcacgcttaccctacctccaatgcgtggttaaagaatcattcagactccaccctccaacgcctctaatgcttcctcacagatccaacgcacacgtcaagatcggaggctacgacatccccaaaggctcaaacgtccacgtgaacgtctgggcggtggctagagacccggccgtgtggaaaaacccattagagtttagaccagagaggttcttggaagaagatgttgacatgaaaggtcatgattttaggctgcttccgttcggagcaggaagaagggtttgtcccggtgcacaacttgggattaacttggtgacttctatgatgagtcatttgcttcaccatttcgtttggacaccgcctcaggggactaaactagatgagattgacatgtctgaaaaccctggactcgttacttacatgcgtgttcctgttcaagccgtggccacgcctcggttgccttctgatctgtataaacgtgtgccttatgaaatgtaaatgtctctgtcttctaccttctgctgttgctgccagtaatgcttaatggtcttgctgtatttcttgtatacttgt

SEQ ID NO: 10 - DNA, 616 bp, Artificial sequence
  RL118F c4h
acgaaatcgacacggttcttggaccaggagttcaagtcacggagcctgagcttcacaagcttccatacctccaagccgtgatcaaggaaacgcttcgtctaagaatggctattcctctcctcgtccctcacatgaacctcaacgacgctaagctcgctggctacgacatcccggcggaaagcaagatcctggtcaacgcctggtggctagcgaacaaccctgagagctggaagaagcctgaagagtttaggcccgagaggttctttgaagaagaggcacacgtggaagcgaacggtaatgactttaggtatgtgccgtttggtgttggacgtagaagctgtccggggattatattggcgttgcctattttgggaatcacgattggtaggatggtacagaacttcgagcttcttcctcctccaggacagtctaaattggatacttctgagaaaggtggacagttcagcttgcacatccttcaccactccaccatcgtaatgaagccaaggtccttttaaaatgacttctttttacactatagtgatgtttgttgaatattccttgtttttgttttgtactattttgtaaggtgttgtgtaaaaagaattcgttccaa

SEQ ID NO: 11 - DNA, 663 bp, Artificial sequence
  p72-139 C3H-C4H fusion
cccgggctgcaggaattcgatactagtggcgcgccagagatgatcaagaacccaagagtgcaacaaaaggtacaagaagagttcgacagagttgttggactagaccgggtcgtaaccgagccagacttctcacgcttaccctacctccaatgcgtggttaaagaatcattcagactccaccctccaacgcctctaatgcttcctcacagatccaacgcacacgtcaagatcggaggctacgacatccccaaaggctcaaacgtccacgtgaacgtctgggcggtggctagagacccggccgtgtggaaaaacccattagagtttagaccagagaggttcttggaagaagatgttgacatgaaaggtcatgattttaggctgcttccgttcggacctgaagagtttaggcccgagaggttctttgaagaagaggcacacgtggaagcgaacggtaatgactttaggtatgtgccgtttggtgttggacgtagaagctgtccggggattatattggcgttgcctattttgggaatcacgattggtaggatggtacagaacttcgagcttcttcctcctccaggacagtctaaattggatacttctgagaaaggtggacagttcagcttgcacatccttcaccactccaccaatttaaatggatcc

SEQ ID NO: 12 - DNA, 1368 bp, Artificial sequence
  rl4637 COMT
acgcgtccgaaaacaaactcttctttcaatcttataaccacaaacaataatgtcaaaccatcttcaagaccccttacccaattaccctaagccggttttaaccgaagaagagcaagaaatcgacgagaaaatggtgagcttgcaagcagagagtatcgtcaacaccgtggctttccccatggttctcaaagctgccttggagcttggcgtcattgacaagatcgctgcagcaggcaacaacatgtggctctcaccgtatgagatagcccgtagtctccctaacaaacccaccaacccggaggcgccagtgttgcttgaccggatgctgcggttactcgtcagccactcgatcttgaagtgccgtatgatagaatgtaaagaaaacggccgaaccggaaatatggagatggtatatgcaaccgaaccggtttgcaagtacttcttgaaagatagtgatggttctggttctcttggttctctgtttatgttgctccacaccgaagtgtttttcaagacttggacaaaccttaaagatgtgatactagaaggaagagatgcatttagctctgcccacggaatgcaaatttttgaatacattaattcagatagacaatttgctgaagtgtttgatcgtgccatgtcagaaccttccatcatgattttgaagaaggttctagaagtttacaaaggatttgaagacgttaacactttggtggatgttggaggaggaagtggcactacattaggtctagtcacttccaagtatcctcatattaagggagttaattttgacttacctcaggttttaaccaatgctccatcttatccaggagtggagcatgtctctggagacatgtttatagaagttccaaaaggagatgcaatatttatgaaatggatactacatgattggtcggacgagctttgtataaagcttctaaaaaattgttggaagagcctaccggaaaatggaaaagtgatcattgtagatttgattacaccaacaaaaccaaagagtggtgacttttcctctaactatatgtttggtatggacatgttgatgctaacacaatgctcaggtggtaaagagagatcgttttcgcagttggagaatttagcatttggttcaggttttcttcggtgtgaagttgtttgtggtgtccattcatattctgttatcgaattccacaaatagatttggaatctacggaaagaaatgatgggccaactgaataaaggatgagaattggtgcttattttaatgattttattgcatgtaacatagttttatggtgttggcttgctcaattttcttttcagcctgtggtgtttgccaagtattaacggagtgtaatgactctgattggt

SEQ ID NO: 13 - DNA, 9706 bp, Artificial sequence
  pGSA1252
  misc_feature (8358)..(8358): n is a, c, g, or t/u, unknown, or other
gaattctgagatttttcaaatcagtgcgcaagacgtgacgtaagtatccgagtcagtttttatttttctactaatttggtcgtttatttcggcgtgtaggacatggcaaccgggcctgaatttcgcgggtattctgtttctattccaactttttcttgatccgcagccattaacgacttttgaatagatacgctgacacgccaagcctcgctagtcaaaagtgtaccaaacaacgctttacagcaagaacggaatgcgcgtgacgctcgcggtgacgccatttcgccttttcagaaatggataaatagccttgcttcctattatatcttcccaaattaccaatacattacactagcatctgaatttcataaccaatctcgatacaccaaatcgaatcgatgagcccagaacgacgcccggccgacatccgccgtgccaccgaggcggacatgccggcggtctgcaccatcgtcaaccactacatcgagacaagcacggtcaacttccgtaccgagccgcaggaaccgcaggagtggacggacgacctcgtccgtctgcgggagcgctatccctggctcgtcgccgaggtggacggcgaggtcgccggcatcgcctacgcgggcccctggaaggcacgcaacgcctacgactggacggccgagtcgaccgtgtacgtctccccccgccaccagcggacgggactgggctccacgctctacacccacctgctgaagtccctggaggcacagggcttcaagagcgtggtcgctgtcatcgggctgcccaacgacccgagcgtgcgcatgcacgaggcgctcggatatgccccccgcggcatgctgcgggcggccggcttcaagcacgggaactggcatgacgtgggtttctggcagctggacttcagcctgccggtaccgccccgtccggtcctgcccgtcaccgagatttgactgcagaatcttggactcccatgttggcaaaggcaaccaaacaaacaatgaatgatccgctcctgcatatggggcggtttgagtatttcaactgccatttgggctgaattgaagacatgctcctgtcagaaattccgtgatcttactcaatattcagtaatctcggccaatatcctaaatgtgcgtggctttatctgtctttgtattgtttcatcaattcatgtaacgtttgcttttcttatgaattttcaaataaattatcagatctctctgccgacagtggtcccaaagatggacccccacccacgaggagcatcgtggaaaaagaagacgttccaaccacgtcttcaaagcaagtggattgatgtgacatctccactgacgtaagggatgacgcacaatcccactatccttcgcaggatctctctgccgacagtggtcccaaagatggacccccacccacgaggagcatcgtggaaaaagaagacgttccaaccacgtcttcaaagcaagtggattgatgtgacatctccactgacgtaagggatgacgcacaatcccactatccttcgcaggatctctctgccgacagtggtcccaaagatggacccccacccacgaggagcatcgtggaaaaagaagacgttccaaccacgtcttcaaagcaagtggattgatgtgacatctccactgacgtaagggatgacgcacaatcccactatccttcgcaagacccttcctctatataaggaagttcatttcatttggagaggacacgctcgagtataagagctcatttttacaacaattaccaacaacaacaaacaacaaacaacattacaattacatttacaattaccatggggcgcgcccaatcgatgatttaaatccccagatgaacatggcatcgtggtgattgatgaaactgctgctgtcggctttaacctctctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaagtgcacgggaatatttcgccactggcggaagcaacgcgtaaactcgacccgacgcgtccgatcacctgcgtcaatgtaatgttctgcgacgctcacaccgataccatcagcgatctctttgatggggatcctcagcttaattaactagtccctagagtcctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaagtgccccaggggctagtatctacgacacaccgagcggcgaactaataacgctcactgaagggaactccggttccccgccggcgcgcatgggtgagattccttgaagttgagtattggccgtccgctctaccgaaagttacgggcaccattcaacccggtccagcacggcggccgggtaaccgacttgctgccccgagaattatgcagcatttttttggtgtatgtgggccccaaatgaagtgcaggtcaaaccttgacagtgacgacaaatcgttgggcgggtccagggcgaattttgcgacaacatgtcgaggctcagcaggacctgcaggcatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcagcttgagcttggatcagattgtcgtttcccgccttcagtttaaactatcagtgtttgacaggatatattggcgggtaaacctaagagaaaagagcgtttattagaataacggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcatgatatatctcccaatttgtgtagggcttattatgcacgcttaaaaataataaaagcagacttgacctgatagtttggctgtgagcaattatgtgcttagtgcatctaatcgcttgagtnaacgccggcgaagcggcgtcggcttgaacgaatttctagctagaggatcgcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccattgtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaactccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccatgatgtttaactttgttttagggcgactgccctgctgcgtaacatcgttgctgctccataacatcaaacatcgacccacggcgtaacgcgcttgctgcttggatgcccgaggcatagactgtaccccaaaaaaacatgtcataacaagaagccatgaaaaccgccactgcgccgttaccaccgctgcgttcggtcaaggttctggaccagttgcgtgacggcagttacgctacttgcattacagcttacgaaccgaacgaggcttatgtccactgggttcgtgcccgaattgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgcc

SEQ ID NO: 14 - DNA, 1211 bp, Artificial sequence
  RL4142 4cl
  misc_feature (1191)..(1191): n is a, c, g, or t/u, unknown, or other
acgagcgtcgctcagcaagtcgacggcgataatccgaatctttacttccacagcgatgacgtcatactctgtgtcttgcccatgttccatatctacgctttgaactcgatcatgctgtgtgggcttagagttggtgcctcgatcttgatcatgccaaagttcgagattaatctgctcttggagctgatacagaggtgtaaagtgacggttgctccgatggttccgccgattgttttggcgatggcgaagtcgccggagacggagaagtatgagttgagctcgattagggttgtgaaatctggtgctgctccgcttggtaaggagcttgaagatgccgtcagtgccaagtttcctaacgccaaactcggtcagggatacggaatgacggaagcaggtccggtgctagcaatgtcgttagggttcgcgaaagagccgtttccagtgaagtcgggagcttgtggtacggtggttagaaacgccgaaatgaaaatcatcgatccagacaccggagactcactttccaaaaacaaacctggagagatttgcatccgtggtcaccagatcatgaaaggttacctcaacaacccggcggctacatcagagaccatagacaaagacggctggcttcacacgggagatatcggattgatcgatgacgacgacgagcttttcattgtcgatagattgaaagaacttatcaagtacaaaggttttcaagtggctccggctgagctagaggctctcctcatcggtcatcaggatatcaccgacgtggccgttgtcgccatgaaagaagaggctgctggtgaagttcccgttgcgtttgttgtgaaatccaaggattcagagttatcagaagatgatgtgaaacaattcgtggcgaaacaggttgtgttttacaagagaatcaacaaagtgttcttcgttgagtccattcctaaagctccgtcagggaagatattgaggaaagacctgagggcaaaactagcaaatgggttggtaaactaaagaaacgaaacgcaaaagatgttccattgtatatacatacaactgtttgacgatcccaatgaaggactagagaaactcatacgaatcaatcaatgcctgtttctttttgtctttgtgttgttgttgtctttggttcatgtaatgaaccttttgtaataaaaggtggtttctttttttttgggnttcatgtaacttgagctgga

SEQ ID NO: 15 - DNA, 1247 bp, Artificial sequence
  rl2419 CCR
  misc_feature (1181)..(1181): n is a, c, g, or t/u, unknown, or other
  misc_feature (1199)..(1199): n is a, c, g, or t/u, unknown, or other
tcttcacaaaccgaagcatatactttagaccgccggaaaaatgccagtcgacgaatcctcaccggccggaaaaaccgtctgcgtcaccggagctggtggatacatagcttcttggattgttaagttactcctcgagagaggctacaccgtcaaaggaaccgtacggaatccagatgatccaaagaatacacatttgagagaactcgaaggagccaaggagagactgattctgtgcaaggcagatcttcaggactacgaggctcttaaagcggcgattgatggctgcgacggcgtgtttcacacggcttctccggtcactgacgatcccgagcaaatggtggagcctgccgtgaacggagccaagttcgtaattaatgcggctgctgaagccaaagtcaagcgcgtggtcatcacctcctcaattggtgcagtctacatggacccgaaccgtgaccctgaagccgtcgttgacgaaagttgttggagtgatcttgagttttgcaaaaacacaaagaattggtattgttacggcaagatggtggcggagcaagccgcgtgggagacggcgaaggagaaaggtgttgaccttgtggttttgaatcccgtgctggtccttggaccgccgctacagccgacgatcaacgccagtctcttccatgtcctcaagtatctaaccggctccgctaagacttacgctaatctgactcaggcttatgtggatgttcgcgatgtcgcgctggcccatgtgctggtctacgaggcaccctcggcctctggacgttatctcctcgcagagagcgcctttcaccgcggcgaagttgttgagattctggctaagttattcccggaatacccacttccgaccaagtgcaaggacgagaagaacccaagagccaagccatacaaattcactaaccaaaagattaaggacttgggcttagagttcacttccaccaagcaaagcctttatgatacagttaagagcttgcaagagaagggtcatcttcctccacctcctcctccttttacatcacaagaatcgagccaaaatggcatcaagatcgagtcttgaatagcttattccctgaagtatcatcctcagcgattgatatatgctttctatttcaaaaaaaacaaacaaacaaacagatgttttgttactgtctctgtttatgtgaaanccttgtttcaaatgtcangtctgaattttgattttgtcttgaaggcttcaatgcttgcttcatgtt

SEQ ID NO: 16 - DNA, 617 bp, Artificial sequence
  p72-168 4cl-ccr fusion
actagtggcgcgccacccggcggctacatcagagaccatagacaaagacggctggcttcacacgggagatatcggattgatcgatgacgacgacgagcttttcattgtcgatagattgaaagaacttatcaagtacaaaggttttcaagtggctccggctgagctagaggctctcctcatcggtcatcaggatatcaccgacgtggccgttgtcgccatgaaagaagaggctgctggtgaagttcccgttgcgtttgttgtgaagacggcgaaggagaaaggtgttgaccttgtggttttgaatcccgtgctggtccttggaccgccgctacagccgacgatcaacgccagtctcttccatgtcctcaagtatctaaccggctccgctaagacttacgctaatctgactcaggcttatgtggatgttcgcgatgtcgcgctggcccatgtgctggtctacgaggcaccctcggcctctggacgttatctcctcgcagagagcgcctttcaccgcggcgaagttgttgagattctggctaagttattcccggaatacccacttccgaccaagtgcaaggacgagaagaacccaagagccaagccatttaaatggatcc

SEQ ID NO: 17 - DNA, 708 bp, Artificial sequence
  cl396r PIKa
gttcccaccggagggttctaagactacgcccccgcatcagtctgtggacttccgctggaaagactactgccctttggtgtttagacggctgagggagcttttcacggtggatcctgctgattacatgttagctatctgtggggacgatgctcttcgggaactgtcttcacctggaaaaagtgggagctttttttacttaactcaagatgacaggtttatgattaagactgtgaagaaatcagaagtcaaggtgctcctacgtatgcttccaaactactacaagcacatctgccaatatgaaaactcacttgtgactaagttctacggtgtacattgcatcaaacctgttggtggccagaagactcggtttattgtcatggggaacttgttctgctcagaatatagaatccagagacggtttgaccttaaagggtcttcccatggacgctccacctccaaacctgaagctgaaattgatgaaacaactactctcaaggaccttgatcttaacttttctttccgtcttcagagaaattggtataaagaactaatgacgcaaattaaacgggactgtgagttcttggaagctgagagaataatggattatagccttttaatcggagttcatttccgagatgacaatacaggagacaagatgggtctttctccattcgttttgagatctggtaagatagagtcatatcaaaa

SEQ ID NO: 18 - DNA, 483 bp, Artificial sequence
  ess1196f pikb
  misc_feature (23)..(23): n is a, c, g, or t/u, unknown, or other
tttttttttttttttttgagtanaatagctttttaccacatgtttctagaatcattttctgatgctggagatcgtctggttagtgaaataacaaagtaggttctgatgttaacaggataatatgaaatacgtggatcagttctatcatcaatgaatcaaatacattactagcttgcaccttacgttatctttaatctcgtcacacaacactcctaattttgtcttttgtcaacaatagcaaagccctgcaaatttattatgcaactctggtttacgataatcgaattcctctccttttttttgttggtttacttctcgatgccttgttgcaggtactgaatcaagtcgtaaccagcagtagtccacttgttatacgcatctgtcagacatggatcatgaagtgtgcagcttcacgttcgctcatctctgggtgaaatcttttccttaggttaccaattggatctcctctgctaaagcaaggca

SEQ ID NO: 19 - DNA, 880 bp, Artificial sequence
  p72-514 PIKa-PIKb fusion
  misc_feature (10)..(10): n is a, c, g, or t/u, unknown, or other
  misc_feature (31)..(31): n is a, c, g, or t/u, unknown, or other
  misc_feature (37)..(37): n is a, c, g, or t/u, unknown, or other
  misc_feature (55)..(55): n is a, c, g, or t/u, unknown, or other
  misc_feature (63)..(63): n is a, c, g, or t/u, unknown, or other
  misc_feature (110)..(110): n is a, c, g, or t/u, unknown, or other
  misc_feature (132)..(132): n is a, c, g, or t/u, unknown, or other
  misc_feature (151)..(151): n is a, c, g, or t/u, unknown, or other
  misc_feature (153)..(153): n is a, c, g, or t/u, unknown, or other
  misc_feature (176)..(176): n is a, c, g, or t/u, unknown, or other
  misc_feature (877)..(877): n is a, c, g, or t/u, unknown, or other
ggataagttntacggtgacatgcatcaaccntttggnggcccagagactcggttnattgtcangggaacttgtctgctcagatatagatccagagacggttggcctaaanggtcttcccaggacgctccacntccaaccctgaagctgaantngatgaaacaactactctcaaggnccttgatcttaacttttctttccgtcttcagagaaattggtataaagaactaatgacgcaaattaaacgggactgtgagttcttggaagctgagagaataatggattatagccttttaatcggagttcatttccgagatgacaatacaggagacaagatgggtgatcgtctggttagtgaaataacaaagtaggttctgatgttaacaggataatatgaaatacgtggatcagttctatcatcaatgaatcaaatacattactagcttgcaccttacgttatctttaatctcgtcacacaacactcctaattttgtcttttgtcaacaatagcaaagccctgcaaatttattatgcaactctggtttacgataatcgaattcctctccttttttttgttggtttacttctcgatgccttgttgcaggtactgaatcaagtcgtagccagcagtagtccacttgttatacgcatctgtacagacatggatcatgaagtgtgcagcttcacgttcgctcatctctgggtgaaatcttttccttaggttaccaattggatctcctctgctaaagcatttaaatggatccgcatctcctctgctaaagcatttaaatggatccgcatcgaattcctgcagcccgggggatccactagttctagagcggccgccaccgcggtggagctccaattcgccctatagangga

SEQ ID NO: 20 - DNA, 894 bp, Artificial sequence
  p72-509 pik
  misc_feature (2)..(2): n is a, c, g, or t/u, unknown, or other
  misc_feature (5)..(5): n is a, c, g, or t/u, unknown, or other
  misc_feature (57)..(57): n is a, c, g, or t/u, unknown, or other
  misc_feature (116)..(116): n is a, c, g, or t/u, unknown, or other
  misc_feature (153)..(153): n is a, c, g, or t/u, unknown, or other
  misc_feature (214)..(214): n is a, c, g, or t/u, unknown, or other
  misc_feature (891)..(892): n is a, c, g, or t/u, unknown, or other
tnccnctttgagtgagctgataccgctcgccgcagccgaacgaccgagcgcagcgantcagtgagcgaggaagcggaagagcgcccaatacgcaaaccgcctctcccccgcgcgtnggccgattccataaatgcagctgccacgacaggtttncccgactggaaagcggccagtgagcgcaacgcaattaatgtgagttagctcactcattagncaccccaggctttacactttatgctcccggctcgtatgttgtgtggaattgtgagcggataacaatttcacacaggaaacagctatgaccatgattacgccaagcgcgcaattaaccctcactaaagggaacaaaagctgggtaccgggccccccctcgaggtcgacggtatcgataagcttgatgctctagaggcgcgccatctcgcaatatgaaaactcacttgtgactaagttctacggtgtacattgcatcaaacctgttggtggccagaagactcggtttattgtcatggggaacttgttctgctcagaatatagaatccagagacggtttgaccttaaagggtcttcccatggacgctccacctccaaacctgaagctgaaattgatgaaacaactactctcaaggaccttgatcttaacttttctttccgtcttcagagaaattggtataaagaactaatgacgcaaattaaacgggactgtgagttcttggaagctgagagaataatggattatagccttttaatcggagttcatttccgagatgacaatacaggagacaagatgggtatttaaatggatccgcatcgaattcctgcagcccgggggatccactagttctagagcggccgccaccgcggtggagctccaattcgccctatagtgaggannga

SEQ ID NO: 21 - DNA, 1246 bp, Artificial sequence
  actin2 promoter
gcaagcttggatccatgtatgcaagagtcagcatatgtataattgattcagaatcgttttgacgagttcggatgtagtagtagccattatttaatgtacatactaatcgtgaatagtgatatgatgaaacattgtatcttattgtataaatatccataaacacatcatgaaagacactttctttcacggtctgaattaattatgatacaattctaatagaaaacgaattaaattacgttgaattgtatgaaatctaattgaacaagccaaccacgacgacgactaacgttgcctggattgactcggtttaagttaaccactaaaaaaacggagctgtcatgtaacacgcggatcgagcaggtcacagtcatgaagccatcaaagcaaaagaactaatccaagggctgagatgattaattagtttaaaaattagttaacacgagggaaaaggctgtctgacagccaggtcacgttatctttacctgtggtcgaaatgattcgtgtctgtcgattttaattatttttttgaaaggccgaaaataaagttgtaagagataaacccgcctatataaattcatatattttcctctccgctttgaattgtctcgttgtcctcctcactttcatcagccgttttgaatctccggcgacttgacagagaagaacaaggaagaagactaagagagaaagtaagagataatccaggagattcattctccgttttgaatcttcctcaatctcatcttcttccgctctttctttccaaggtaataggaactttctggatctactttatttgctggatctcgatcttgttttctcaatttccttgagatctggaattcgtttaatttggatctgtgaacctccactaaatcttttggttttactagaatcgatctaagttgaccgatcagttagctcgattatagctaccagaatttggcttgaccttgatggagagatccatgttcatgttacctgggaaatgatttgtatatgtgaattgaaatctgaactgttgaagttagattgaatctgaacactgtcaatgttagattgaatctgaacactgtttaagttagatgaagtttgtgtatagattcttcgaaactttaggatttgtagtgtcgtacgttgaacagaaagctatttctgattcaatcagggtttatttgactgtattgaactctttttgtgtgtttgcagctcataaaaaatggctgaggctgatctcgagtctaga

SEQ ID NO: 22 - DNA, 878 bp, Artificial sequence
  p72-507 ip6k
  misc_feature (2)..(2): n is a, c, g, or t/u, unknown, or other
  misc_feature (14)..(14): n is a, c, g, or t/u, unknown, or other
  misc_feature (548)..(548): n is a, c, g, or t/u, unknown, or other
  misc_feature (681)..(681): n is a, c, g, or t/u, unknown, or other
  misc_feature (748)..(748): n is a, c, g, or t/u, unknown, or other
  misc_feature (774)..(774): n is a, c, g, or t/u, unknown, or other
  misc_feature (794)..(794): n is a, c, g, or t/u, unknown, or other
  misc_feature (805)..(805): n is a, c, g, or t/u, unknown, or other
  misc_feature (822)..(822): n is a, c, g, or t/u, unknown, or other
  misc_feature (828)..(828): n is a, c, g, or t/u, unknown, or other
  misc_feature (830)..(830): n is a, c, g, or t/u, unknown, or other
  misc_feature (834)..(836): n is a, c, g, or t/u, unknown, or other
  misc_feature (854)..(854): n is a, c, g, or t/u, unknown, or other
  misc_feature (864)..(864): n is a, c, g, or t/u, unknown, or other
  misc_feature (878)..(878): n is a, c, g, or t/u, unknown, or other
gnggattgtaatancactcactataggggcgaattgggagctccaccgcggtggcggccgctctagaactagaggcgcgccttccagaacacatccatagatacttccctgtctatcacggcactcaagctgtcgaaggctctgatggagcagccatgattgtcctggagaatcttctctcccagtactctaagccatcggtgatggatgtcaagatgggctcaagaacatggtaccctgaagcatccgaagaatacatccaaaaatgcttaaggaaaggcgctaggagcaccacggtctcctccggtttcaggatctctggtttcgaagtgtatgatcatttaaatggatccccatcaaagagatcgctgatggtatcggtgtgagcgtcgcagaacattacattgacgcaggtgatcggacgcgtcgggtcgagtttacgcgttgcttccgccagtggcgcgaaatattcccgtgcaccttgcggacgggtatccggttcgttggcaatactccacatcaccacgcttgggtggtttttgtcacgngctatcagctctttaatcgcctgtaagtgcgcttgctgagtttccccgttgactgcctcttcgctgtacagttctttcggcttgttgcccgcttcgaaaccaatgcctaaagagaggttaaagccgacagcancagtttcatcaatcaccaccgatgccatgttcatctggggatttaaaatgatcatacacttcgaaancagaaaatcctgaaaccggaggaganccgtggtgctcctagcgccntttcctttaancattttttgggatgtanttcttncnggannnttttcaggggtaaccatnttttttgaanccccatctttgaan

SEQ ID NO: 23 - DNA, 717 bp, Artificial sequence
  rl1344r ip3k
  misc_feature (641)..(641): n is a, c, g, or t/u, unknown, or other
ctaagattctgtatcatctcttcgatctgatccgtgcagaatctcgcattgataaaggttttgatccctggggcatgaatgttaacctaacttcgtcaccttcgaagcagagcaacagcgcaagatggcagattcaatcaaggagaggtacgtagtcgggtacgctctcgcggagaagaagcagaacagtttcatccagccttcgttgatcgggcactcgaggcaacgaggcattgatctaatcaaactcgatccagcgaaatctttgttagagcaagggaacctcgatttcgtaatccacaagctttacgacgtcgtttggaaggagaatctcagtcagtttcgcgagaagtgtccatgtgtccccgtcgttgattctcccgaggcgatcgagaggttgcacaatagggtttcgatgctcgaagtgatcactcagctaacgtttcccgtctccgaaagcgagtgttttggagttccgaagcaggttgtggtgatggatgagaacgttttgagcggtgatggagctttaggggagcttgagtttccggtgatagctaagcccttggacgctgatgggagcgcgaagtctcacaagatgttcttgatctacgatcaagaagggatgaagatactcaaagctncgattgtgctgcaggagtttgtgaatcacggtggtgtgatcttcaaggtctatgttgttggagactacgtgaagtg

SEQ ID NO: 24 - DNA, 888 bp, Artificial sequence
  p72-512  ip6k-ip3k
  misc_feature (676)..(676): n is a, c, g, or t/u, unknown, or other
  misc_feature (681)..(681): n is a, c, g, or t/u, unknown, or other
  misc_feature (684)..(684): n is a, c, g, or t/u, unknown, or other
  misc_feature (702)..(702): n is a, c, g, or t/u, unknown, or other
  misc_feature (731)..(731): n is a, c, g, or t/u, unknown, or other
  misc_feature (746)..(746): n is a, c, g, or t/u, unknown, or other
  misc_feature (750)..(750): n is a, c, g, or t/u, unknown, or other
  misc_feature (758)..(758): n is a, c, g, or t/u, unknown, or other
  misc_feature (776)..(776): n is a, c, g, or t/u, unknown, or other
  misc_feature (783)..(783): n is a, c, g, or t/u, unknown, or other
  misc_feature (802)..(802): n is a, c, g, or t/u, unknown, or other
  misc_feature (808)..(808): n is a, c, g, or t/u, unknown, or other
  misc_feature (810)..(810): n is a, c, g, or t/u, unknown, or other
  misc_feature (845)..(845): n is a, c, g, or t/u, unknown, or other
  misc_feature (860)..(860): n is a, c, g, or t/u, unknown, or other
  misc_feature (864)..(864): n is a, c, g, or t/u, unknown, or other
  misc_feature (884)..(884): n is a, c, g, or t/u, unknown, or other
  misc_feature (888)..(888): n is a, c, g, or t/u, unknown, or other
tccttctatagggcgaattggagctccaccgcggtggcggccgctctagaactagtggatcccccgggctgcaggaattcgatcctctagaggcgcgccttccagaacacatccatagatacttccctgtctatcacggcactcaagctgtcgaaggctctgatggagcagccacaattgtcctggagaatcttctctcccagtactctaagccatcggtgatggatgtcaagatgggctcaagaacatggtaccctgaagcatccgaagaatacatccaaaaatgcttaaggaaaggcgctaggagcaccacggtctcctccggtttcaggacctctggtttcgaagtgtatgatcatttaaatggtcgcggagaagaagcagaacagtttcatccagccttcgttgatcgggcactcgaggcaacgaggcatttatctaatcaaactcgatccagcgaaatctttgttagagcaagggaacctcgatttcgtaatccacaagctttacgacgtcgtttggaaggagaatctcagtcagtttcgcgagaagtgtccatgtgtccccgtcgttgattctcccgaggcgatcgagaggttgcacaatagggtttcgatgctcgaagtgatcactcagctaacgtttcccgtctccgaaagcgagtgttttggagttncgaancangttgtggtgatggatganaacgttttgagcggtggatggagctttangggagcttgagtttncggngatagctnagcccattttaatggatncgcatcnaggcttatcgataccgtcnaactcnangggggggccggtaccacttttgttccctttatggngggtaaatgcgcgcntggngaatcatgggcatagctggnttcn

SEQ ID NO: 25 - DNA, 43 bp, Artificial sequence
  OMT-F5 primer
gcactagtggcgcgccattgcattatgctagctcacaaccctg

SEQ ID NO: 26 - DNA, 42 bp, Artificial sequence
  OMT R6 primer
gcggatccatttaaataacaaagacggtgaagtagacgtacc

SEQ ID NO: 27 - DNA, 22 bp, Artificial sequence
  SNG2-FI primer
gcggaagcctttaagactattg

SEQ ID NO: 28 - DNA, 24 bp, Artificial sequence
  SNG2-R2 primer
catgggatgggacttatttcagat

SEQ ID NO: 29 - DNA, 48 bp, Artificial sequence
  SCT-F7 primer
gcactagtggcgcgccatcagtgtatctcagagatatacatagagcag

SEQ ID NO: 30 - DNA, 43 bp, Artificial sequence
  SCT-R8 primer
gcggatccatttaaatagcagcttggaggaggcaacgatgatg

SEQ ID NO: 31 - DNA, 39 bp, Artificial sequence
  FAH-F7 primer
gcactagtggcgcgccgctcgtgaaggcccgtaatgacc

SEQ ID NO: 32 - DNA, 31 bp, Artificial sequence
  FAH-R9 primer
ctgagatacatgcggtttcgtgtaggaggag

SEQ ID NO: 33 - DNA, 36 bp, Artificial sequence
  SCT-F11 primer
gaaaccgcatgtatctcagagatatacatagagcag

SEQ ID NO: 34 - DNA, 43 bp, Artificial sequence; identical to SEQ ID NO: 30
  SCT-R8 primer

SEQ ID NO: 35 - DNA, 45 bp, Artificial sequence
  cruc-F3 primer
gcagatctaagcttttggccctttaattatgctctctttctaatc

SEQ ID NO: 36 - DNA, 46 bp, Artificial sequence
  curc-R4 primer
gcgagctctctagaattgtgtgtgttttggtgatagatggatgaag

SEQ ID NO: 37 - DNA, 35 bp, Artificial sequence
  OMT-F1 5′ primer
gcggatccattgcattatgctagctcacaaccctg

SEQ ID NO: 38 - DNA, 34 bp, Artificial sequence
  OMT-R2 5′ primer
gctctagaaacaaagacggtgaagtagacgtacc

SEQ ID NO: 39 - DNA, 34 bp, Artificial sequence
  COMT-F1 primer
gcgagctctcttcaagaccccttacccaattacc

SEQ ID NO: 40 - DNA, 32 bp, Artificial sequence
  COMT-R2 primer
gctctagagtgggtttgttagggagactacgg

SEQ ID NO: 41 - DNA, 44 bp, Artificial sequence
  SAMS-F5 primer
gcactagtggcgcgccagaacgggacttgcgcttggcttagacc

SEQ ID NO: 42 - DNA, 45 bp, Artificial sequence
  SAMS-R6 primer
gcggatccatttaaatgaacacagacaacggctcagggacaccaa

SEQ ID NO: 43 - DNA, 30 bp, Artificial sequence
  CCR-F8 primer
cgtttgttgtgaagacggcgaaggagaaag

SEQ ID NO: 44 - DNA, 31 bp, Artificial sequence
  FAH-R10 primer
gcataatgcatgcggtttcgtgtaggaggag

SEQ ID NO: 45 - DNA, 35 bp, Artificial sequence
  OMT1-F7 primer
cgaaaccgcatgcattatgctagctcacaaccctg

SEQ ID NO: 46 - DNA, 42 bp, Artificial sequence; identical to SEQ ID NO: 26
  OMT-R6 primer fusion

SEQ ID NO: 47 - DNA, 41 bp, Artificial sequence
  C3H-F3
cgactagtggcgcgccagagatgatcaagaacccaagagtg

SEQ ID NO: 48 - DNA, 28 bp, Artificial sequence
  C3H-R4 primer
aactcttcagctccgaacggaagcagcc

SEQ ID NO: 49 - DNA, 32 bp, Artificial sequence
  C4H-F3 primer
ccgttcggacctgaagagtttaggcccgagag

SEQ ID NO: 50 - DNA, 38 bp, Artificial sequence
  C4H-R4 primer
gcggatccatttaaattggtggagtggtgaaggatgtg

SEQ ID NO: 51 - DNA, 39 bp, Artificial sequence
  4CL-F5 primer
gcactagtggcgcgccacccggcggctacatcagagacc

SEQ ID NO: 52 - DNA, 40 bp, Artificial sequence
  4CL-R6 primer
ctggatccatttaaatcacaacaaacgcaacgggaacttc

SEQ ID NO: 53 - DNA, 41 bp, Artificial sequence
  CCR-F5 primer
acactagtggcgcgccagacggcgaaggagaaaggtgttga

SEQ ID NO: 54 - DNA, 42 bp, Artificial sequence
  CCR-R6 primer
caggatccatttaaatggcttggctcttgggttcttctcgtc

SEQ ID NO: 55 - DNA, 42 bp, Artificial sequence; identical to SEQ ID NO: 54
  CCR-R6 primer

SEQ ID NO: 56 - DNA, 23 bp, Artificial sequence
  4CL-R7 primer
cttcgccgtcttcacaacaaacg

SEQ ID NO: 57 - DNA, 30 bp, Artificial sequence; identical to SEQ ID NO: 43
  Plasmid p72-164

SEQ ID NO: 58 - DNA, 36 bp, Artificial sequence
  forward primer PIK-F11
gctctagaggcgcgccatctcgcaatatgaaaactc

SEQ ID NO: 59 - DNA, 30 bp, Artificial sequence
  reverse primer PIK-R20
ccagacgatcacccatcttgtctcctgtat

SEQ ID NO: 60 - DNA, 28 bp, Artificial sequence
  forward primer PIK-F19
caagatgggtgatcgtctggttagtgaa

SEQ ID NO: 61 - DNA, 33 bp, Artificial sequence
  reverse primer PIK-R16
gcggatccatttaaatgctttagcagaggagat

SEQ ID NO: 62 - DNA, 29 bp, Artificial sequence
  forward primer act-F5
gcaagcttggatccatgtatgcaagagtc

SEQ ID NO: 63 - DNA, 29 bp, Artificial sequence
  reverse primer act-R6
gctctagactcgagatcagcctcagccat

SEQ ID NO: 64 - DNA, 36 bp, Artificial sequence
  reverse primer PIK-R12
gcggatccatttaaatacccatcttgtctcctgtat

SEQ ID NO: 65 - DNA, 34 bp, Artificial sequence
  forward primer PIK-F5\IP6K-F5
cctctagaggcgcgccttccagaacacatccata

SEQ ID NO: 66 - DNA, 36 bp, Artificial sequence
  forward primer IP6K-F5
cgggatccatttaaatgatcatacacttcgaaacca

SEQ ID NO: 67 - DNA, 24 bp, Artificial sequence
  reverse primer IP6K-R11
ccatttaaatgatcatacacttcg

SEQ ID NO: 68 - DNA, 28 bp, Artificial sequence
  forward primer IP3K-F12
catttaaatggtcgcggagaagaagcag

SEQ ID NO: 69 - DNA, 40 bp, Artificial sequence
  reverse primer IP3K-R10
gcggatccatttaaatgggcttagctatcaccggaaactc